(19) United States
(12) Patent Application Publication
(10) Pub. No.: US 2025/0236872 A1
(43) Pub. Date: Jul. 24, 2025

(54) TARGETED TRANS-SPLICING USING CRISPR/CAS13

(71) Applicant: Massachusetts Institute of Technology, Cambridge, MA (US)

(72) Inventor: Jacob Borrajo, Cambridge, MA (US)

(21) Appl. No.: 19/171,510

(22) Filed: Apr. 7, 2025

Related U.S. Application Data
(63) Continuation of application No. 18/449,850, filed on Aug. 15, 2023, now Pat. No. 12,297,433.
Application No. 18/449,850 is a continuation of application No. 16/994,230, filed on Aug. 14, 2020, now Pat. No. 11,767,528.
(60) Provisional application No. 62/985,633, filed on Mar. 5, 2020.
(60) Provisional application No. 62/888,210, filed on Aug. 16, 2019.

(51) Int. Cl. C12N 15/113; C12N 9/22

(52) U.S. Cl. CPC C12N 15/113; C12N 9/22; C12N 2310/20

(57) ABSTRACT

This disclosure provides compositions and methods of using these compositions to mediate a targeted trans-splicing event on a pre-mRNA in a cell.

### CROSS-REFERENCE TO RELATED APPLICATIONS

This application is a continuation of pending U.S. patent application Ser. No. 18/449,850, filed Aug. 15, 2023, which is a continuation of U.S. patent application Ser. No. 16/994,230, filed Aug. 14, 2020, now U.S. Pat. No. 11,767,528, which claims priority to U.S. Provisional Application No. 62/888,210, filed Aug. 16, 2019, and U.S. Provisional Application No. 62/985,633, filed Mar. 5, 2020. Each of these applications is incorporated by reference in its entirety herein.

### STATEMENT REGARDING FEDERALLY SPONSORED RESEARCH

This invention was made with Government support under Grant No. HL141005 awarded by the National Institutes of Health (NIH). The Government has certain rights in the invention.

### REFERENCE TO SEQUENCE LISTING SUBMITTED ELECTRONICALLY

The instant application contains a Sequence Listing which has been submitted electronically in XML format and is hereby incorporated by reference in its entirety. The XML file, created on Mar. 14, 2025, is named 763170_083474-007USCON3_ST26.xml and is 284,727 bytes in size.

### FIELD

The present disclosure relates to the field of CRISPR/Cas-mediated trans-splicing of nucleic acids for the treatment of disease.

### BACKGROUND

Trans-splicing is a form of RNA processing where exons from two different primary pre-mRNA transcripts are joined end to end and ligated. This process is a promising strategy to treat various diseases using the spliceosome ribonucleoprotein complex. However, the efficiency and the specificity of trans-splicing of the current systems need to be improved upon to provide a more effective therapeutic strategy.

CRISPR/Cas systems have been used to edit genomic loci to treat diseases and disorders. This leads to a permanent edit to the DNA. The present invention provides a CRISPR/Cas system to target a trans-splicing element to a desired pre-mRNA sequence, and allows for the option of transient RNA repair while improving both the specificity and the efficiency of trans-splicing events.

### SUMMARY

This disclosure provides methods and compositions for mediating a targeted trans-splicing event on a pre-mRNA in a cell. In one aspect, targeted trans-splicing system comprises: a nucleic acid-targeting CRISPR/Cas system; a nucleic acid guide; a specific RNA-binding domain; and a repair template comprising a splice donor and/or acceptor and an RNA sequence that hybridizes under physiological conditions to the specific RNA-binding domain.

In various embodiments, the nucleic acid guide is a RNA guide. In various embodiments, the nucleic acid guide is a DNA guide. In various embodiments the trans-splicing system comprises one nucleic acid guide. In various embodiments, the trans-splicing system comprises more than one nucleic acid guide. In various embodiments, the nucleic acid guides recognize multiple targets. In various embodiments, the nucleic acid guide targets a splice acceptor (SA) site. In various embodiments, the nucleic acid guide targets a splice donor (SD) site. In various embodiments, the nucleic acid guide targets a region near a splice site. In various embodiments, the nucleic acid guide targets a region within 200 nucleotides of a splice site. In various embodiments, the nucleic acid guide targets a region less than or equal to 100 nucleotides from a splice site. In various embodiments, the more than one nucleic acid guides target one nucleic acid of interest. In various embodiments, the more than one nucleic acid guides target multiple nucleic acids of interest.

In various embodiments, the CRISPR/Cas system comprises a Cas13 polypeptide. In various embodiments, the Cas13 is a nuclease-inactive Cas13 polypeptide (dCas13). In various embodiments, the Cas13 is a nuclease-active Cas13 polypeptide. In various embodiments, the specific RNA-binding domain comprises a viral protein. In various embodiments, the viral protein is a MS2 binding protein.

In various embodiments, the viral protein is a λN protein. In various embodiments, the viral protein is a PP7 coat protein. In various embodiments, the viral protein is a QBeta coat protein.

In various embodiments, the viral protein is covalently bound to the CRISPR/Cas system. In various embodiments, the viral protein is not covalently bound to the CRISPR/Cas system. In various embodiments, the viral protein and CRISPR/Cas system is a fusion protein.

In one embodiment, the trans-splicing system comprises one repair template. In one embodiment, the trans-splicing system comprises more than one repair template. In various embodiments, the repair template is introduced as DNA. In various embodiments, the repair template is delivered as RNA.

In various embodiments, the repair template is expressed as RNA. In various embodiments, the repair template comprises a splice acceptor. In various embodiments, the repair template comprises a splice donor. In various embodiments, the repair template comprises one or more splice sites. In various embodiments, the repair template comprises an exon. In various embodiments, the repair template comprises more than one exon. In various embodiments, the repair template comprises an intron. the repair template comprises more than one intron. In various embodiments, the repair template comprises one or multiple sequences that hybridize to the target nucleic acid of interest. In various embodiments, at least one portion of the repair template comprises a ms2 hairpin that specifically binds to the MS2 binding protein. In various embodiments, the repair template comprises a boxB hairpin that specifically binds to the λN protein. In various embodiments, the repair template comprises a PP7 hairpin that specifically binds to the PP7 coat protein. In various embodiments, the repair template comprises a QBeta hairpin that specifically binds to the QBeta coat protein.

In various embodiments, the targeted trans-splicing system further comprises a cell. In various embodiments, at least one portion of the trans-splicing system is introduced into the cell.

In various embodiments, expression and/or activity of at least one portion of the trans-splicing system is transient. In various embodiments, the activity of at least one portion of the trans-splicing system is regulated by a small molecule. In various embodiments, the small molecule is selected from abscisic acid (ABA), rapamycin (or rapalog), FK506, Cyclosporine A, FK1012, Gibberellin3-AM, FKCsA, AP1903/AP20187, and Auxin. In various embodiments, the at least one portion is a Cas13 protein. In various embodiments, the Cas13 protein further comprises a small molecule binding domain. In various embodiments, the Cas13 protein and the small molecule binding domain are linked by a glycine-serine linker. In various embodiments, the small molecule binding domain is an ABA-binding domain. In various embodiments, the ABA-binding domain comprises an ABI1 polypeptide.

In various embodiments, the targeted trans-splicing system further comprises a viral protein. In various embodiments, the viral protein further comprises a small molecule binding domain. In various embodiments, the viral protein and the small molecule binding domain are linked by a glycine-serine linker. In various embodiments, the small molecule binding domain is an ABA-binding domain. In various embodiments, the ABA-binding domain comprises a PYL1 polypeptide. In various embodiments, the addition of ABA induces targeted trans-splicing of a target pre-mRNA.

In various embodiments, delivery of at least one portion of the trans-splicing system to the cell is viral. In various embodiments, the virus is a retrovirus, adenovirus, adeno-associated virus, herpes simplex virus, anellovirus, or baculovirus. In various embodiments, delivery of at least one portion of the trans-splicing system to a cell is non-viral. In various embodiments, the non-viral delivery system is selected from a cationic lipid vehicle, electroporation, calcium phosphate transfection, mechanical transfection, and nanoparticle delivery.

In various embodiments, the CRISPR/Cas system is targeted to a nucleic acid. In various embodiments, the nucleic acid is RNA. In various embodiments, the nucleic acid is DNA. In various embodiments, the CRISPR/Cas system is associated with RNA in the cell. In various embodiments, the RNA is a pre-mRNA. In various embodiments, the CRISPR/Cas system is associated with DNA in the cell. In various embodiments, the CRISPR/Cas system is not associated with DNA in the cell. In various embodiments, the CRISPR/Cas system mediates trans-splicing of pre-mRNA in the nucleus of the cell. In various embodiments, the CRISPR/Cas system mediates trans-splicing of pre-mRNA in the cytoplasm of the cell.

In various embodiments, the trans-splicing occurs in dividing cells. In various embodiments, the trans-splicing occurs in post-mitotic cells. In various embodiments, post-mitotic cells are neurons, myocytes, or adipocytes.

In various embodiments, expression of at least one portion of the trans-splicing system is inducible. In various embodiments, activity of at least one portion of the trans-splicing system is inducible.

In another aspect, provided herein is a method of mediating a targeted trans-splicing event on a pre-mRNA in a cell, the method comprises introducing at least one portion of a targeted trans-splicing CRISPR/Cas system into the cell. In one aspect, the CRISPR/Cas system comprises: a nucleic acid-targeting CRISPR/Cas system; a nucleic acid guide that specifically hybridizes to a nucleic acid locus of interest; a specific RNA-binding domain; and a repair template comprising a splice donor and/or a splice acceptor and an RNA sequence that hybridizes under physiological conditions to the specific RNA-binding domain.

In one embodiment, the nucleic acid guide comprises RNA. In another embodiment, the nucleic acid guide comprises DNA.

In one embodiment, a single nucleic acid guide is introduced into the cell. In another embodiment, multiple nucleic acid guides are introduced into the cell.

In one embodiment, the multiple nucleic acid guides target one nucleic acid of interest. In another embodiment, the multiple nucleic acid guides target more than one nucleic acid of interest.

In various embodiments, the nucleic acid guide targets a splice acceptor (SA) site. In other embodiments, the nucleic acid guide targets a splice donor (SD) site.

In various embodiments, the nucleic acid guide targets a region near the splice site.

In various embodiments, the nucleic acid guide targets a region within 200 nucleotides of the splice site. In other embodiments, the nucleic acid guide targets a region less than or equal to 100 nucleotides of the splice site.

In various embodiments, a plurality of nucleic acid guides is delivered to a plurality of cells. In various embodiments, the CRISPR/Cas system comprises a Cas13 polypeptide.

In various embodiments, the Cas13 polypeptide is a Cas13b polypeptide. In various embodiments, the Cas13b polypeptide is a nuclease-inactive Cas13b (dCas13b). In various embodiments, the Cas13b polypeptide is a nuclease-active Cas13b.

In various embodiments, the specific RNA-binding domain comprises a viral protein. In various embodiments, the viral protein is a MS2 binding protein. In various embodiments, the viral protein is a λN protein.

In various embodiments, the viral protein is a PP7 coat protein. In various embodiments, the viral protein is a QBeta coat protein.

In various embodiments, the viral protein is covalently bound to the CRISPR/Cas system. In various embodiments, the viral protein is not covalently bound to the CRISPR/Cas system. In various embodiments, the viral protein and CRISPR/Cas system is a fusion protein.

In various embodiments, the trans-splicing system comprises one splice acceptor repair template. In various embodiments, the trans-splicing system comprises more than one splice acceptor repair template. In various embodiments, the trans-splicing system comprises one splice donor repair template.

In various embodiments, the trans-splicing system comprising more than one splice donor repair template.

In various embodiments, the repair template comprises a ms2 hairpin that specifically binds to the MS2 binding protein. In various embodiments, the repair template comprises a boxB hairpin that specifically binds to the λN protein.

In various embodiments, the repair template comprises a PP7 hairpin that specifically binds to the PP7 coat protein. In various embodiments, the repair template comprises a QBeta hairpin that specifically binds to the QBeta coat protein.

In various embodiments, expression of at least one portion of the trans-splicing system is transient. In various embodiments, the activity of at least one portion of the trans-splicing system is transient. In various embodiments, the activity of at least one portion of the trans-splicing system is regulated by a small molecule. In various embodiments, the small molecule is selected from abscisic acid (ABA), rapamycin (or rapalog), FK506, Cyclosporine A, FK1012, Gibberellin3-AM, FKCsA, AP1903/AP20187, and Auxin.

In various embodiments, at least one portion of the trans-splicing system comprises a Cas13 polypeptide. In various embodiments, the Cas13 polypeptide further comprises a small molecule binding domain.

In various embodiments, the Cas13 polypeptide and the small molecule binding domain are linked by a glycine-serine linker. In various embodiments, the small molecule binding domain is an ABA-binding domain.

In various embodiments, the ABA-binding domain comprises an ABI1 polypeptide. In various embodiments, the method further comprises a viral protein. In various embodiments, the viral protein further comprises a small molecule binding domain. In various embodiments, the viral protein and the small molecule binding domain are linked by a glycine-serine linker.

In various embodiments, the small molecule binding domain is an ABA-binding domain.

In various embodiments, the ABA-binding domain comprises a PYL1 polypeptide.

In various embodiments, the addition of ABA induces targeted trans-splicing of a target pre-mRNA.

In various embodiments, delivery of at least one portion of the trans-splicing system to a cell is viral.

In various embodiments, the virus is a retrovirus, adenovirus, adeno-associated virus, herpes simplex virus, anellovirus or baculovirus.

In various embodiments, delivery of at least one portion of the trans-splicing system to a cell is non-viral.

In various embodiments, the non-viral delivery system is selected from a cationic lipid vehicle, electroporation, calcium phosphate transfection, mechanical transfection, and nanoparticle delivery.

In various embodiments, the CRISPR/Cas system associates with RNA in the cell.

In various embodiments, the RNA is a pre-mRNA.

In various embodiments, the CRISPR/Cas system associates with DNA in the cell.

In various embodiments, the CRISPR/Cas system does not associate with DNA in the cell.

In various embodiments, the CRISPR/Cas system mediates trans-splicing of pre-mRNA in the nucleus of the cell.

In various embodiments, the CRISPR/Cas system mediates trans-splicing of pre-mRNA in the cytoplasm of the cell.

In various embodiments, the CRISPR/Cas system mediates trans-splicing of a pre-mRNA at intron-exon junctions.

In various embodiments, wherein the CRISPR/Cas system mediates trans-splicing of a pre-mRNA at exon-intron junctions.

In various embodiments, the splice repair template does not comprise a splice acceptor.

In various embodiments, the splice repair template does not comprise a splice donor.

In various embodiments, the splice repair template comprises both a splice acceptor and a splice donor.

In various embodiments, the CRISPR/Cas system mediates trans-splicing of a pre-mRNA at the 5′ end of the pre-mRNA. In various embodiments, the CRISPR/Cas system mediates trans-splicing of a pre-mRNA at the 3′ end of the pre-mRNA. In various embodiments, the CRISPR/Cas system mediates trans-splicing of a pre-mRNA at an internal site within a pre-mRNA. In various embodiments, the CRISPR/Cas system mediates trans-splicing of a pre-mRNA within the 3′ untranslated region (UTR). In various embodiments, the CRISPR/Cas system mediates trans-splicing of a pre-mRNA within the 5′ untranslated region (UTR).

In various embodiments, the cell is a dividing cell. In various embodiments, the cell is a post-mitotic cell.

In various embodiments, the post-mitotic cell is selected from the group consisting of a neuron, myocyte, and adipocyte.

In various embodiments, expression of at least one portion of the trans-splicing system is inducible.

In various embodiments, activity of at least one portion of the trans-splicing system is inducible.

In various embodiments, the cell is in a subject suffering from a disease or disorder.

In various embodiments, the disease or disorder is a neurodegenerative, neurological, or neuromuscular disease or disorder.

In various embodiments, the neurodegenerative, neurological, or neuromuscular disease or disorder is selected from the group consisting of spinal muscular atrophy, Rett Syndrome, Angelman syndrome, Parkinson's disease, Alzheimer's disease, Huntington's disease, frontotemporal dementia, lysosomal storage diseases, multiple sclerosis, and amyotrophic lateral sclerosis (ALS).

In yet another aspect, disclosed herein is a targeted trans-splicing system comprising: a nucleic acid binding protein domain; a specific RNA-binding domain; and a repair template comprising a splice donor and/or a splice acceptor and an RNA sequence that hybridizes under stringent conditions to the specific RNA-binding domain.

In various embodiments, the nucleic acid binding protein domain targets a splice acceptor (SA) site. In various embodiments, the nucleic acid binding protein domain targets a splice donor (SD) site. In various embodiments, the specific RNA-binding domain comprises a viral protein. In various embodiments, the viral protein is a MS2 binding protein. In various embodiments, the viral protein is a λN protein. In various embodiments, the viral protein is a PP7 coat protein. In various embodiments, the viral protein is a QBeta coat protein. In various embodiments, the viral protein is covalently bound to the nucleic acid binding protein domain. In various embodiments, the viral protein is not covalently bound to the nucleic acid binding protein domain. In various embodiments, the viral protein and nucleic acid binding protein domain is a fusion protein.

In various embodiments, the trans-splicing system comprises one splice acceptor repair template. In various embodiments, the trans-splicing system comprises more than one splice acceptor repair template. In various embodiments, the trans-splicing system comprises one splice donor repair template. In various embodiments, the trans-splicing system comprises more than one splice donor repair template. In various embodiments, the trans-splicing system comprises a repair template comprising a splice donor and/or a splice acceptor. In various embodiments, at least one portion of the repair template comprises a ms2 hairpin that specifically binds to the MS2 binding protein. In various embodiments, the splice repair template comprises a boxB hairpin that specifically binds to the λN protein. In various embodiments, the splice repair template comprises a PP7 hairpin that specifically binds to the PP7 coat protein. In various embodiments, the splice repair template comprises a QBeta hairpin that specifically binds to the QBeta coat protein.

In various embodiments, the targeted trans-splicing system further comprises a cell. In various embodiments, at least one portion of the trans-splicing system is introduced into the cell.

In various embodiments, expression and/or activity of at least one portion of the trans-splicing system is transient. In various embodiments, the activity of at least one portion of the trans-splicing system is regulated by a small molecule. In various embodiments, the small molecule is selected from abscisic acid (ABA), rapamycin (or rapalog), FK506, Cyclosporine A, FK1012, Gibberellin3-AM, FKCsA, AP1903/AP20187, and Auxin. In various embodiments, at least one portion is the nucleic acid binding protein domain. In various embodiments, the nucleic acid binding protein domain further comprises a small molecule binding domain. In various embodiments, the nucleic acid binding protein domain and the small molecule binding domain are linked by a glycine-serine linker. In various embodiments, the small molecule binding domain is an ABA-binding domain. In various embodiments, the ABA-binding domain comprises an ABI1 polypeptide. In various embodiments, the trans-splicing system further comprises a viral protein. In various embodiments, the viral protein further comprises a small molecule binding domain. In various embodiments, the viral protein and the small molecule binding domain are linked by a glycine-serine linker. In various embodiments, the small molecule binding domain is an ABA-binding domain. In various embodiments, the ABA-binding domain comprises a PYL1 polypeptide. In various embodiments, the addition of ABA induces targeted trans-splicing of a target pre-mRNA.

In various embodiments, delivery of at least one portion of the trans-splicing system to the cell is viral. In various embodiments, the virus is a retrovirus, adenovirus, adeno-associated virus, herpes simplex virus, anellovirus, or baculovirus. In various embodiments, delivery of at least one portion of the trans-splicing system to a cell is non-viral. In various embodiments, the non-viral delivery system is selected from a cationic lipid vehicle, electroporation, calcium phosphate transfection, mechanical transfection, and nanoparticle delivery.

In various embodiments, the nucleic acid binding protein domain is targeted to DNA. In various embodiments, the nucleic acid binding protein domain is targeted to RNA. In various embodiments, the nucleic acid binding protein domain is associated with RNA in the cell. In various embodiments, the RNA is a pre-mRNA. In various embodiments, the nucleic acid binding protein domain is associated with DNA in the cell. In various embodiments, the nucleic acid binding protein domain is not associated with DNA in the cell.

In various embodiments, the system mediates trans-splicing of pre-mRNA in the nucleus of the cell. In various embodiments, the system mediates trans-splicing of pre-mRNA in the cytoplasm of the cell. In various embodiments, the system mediates trans-splicing of a pre-mRNA at intron-exon junctions. In various embodiments, the nucleic acid binding protein domain mediates trans-splicing of a pre-mRNA at exon-intron junctions. In various embodiments, the splice repair template does not comprise a splice donor. In various embodiments, the splice repair template does not comprise a splice acceptor. In various embodiments, the splice repair template comprises both a splice acceptor and a splice donor.

In various embodiments, the system comprises multiple splice templates. In various embodiments, the multiple splice templates comprise splice repair templates that comprise a splice acceptor and splice repair templates that comprise a splice donor. In various embodiments, at least some of the multiple splice repair templates comprise a splice acceptor and a splice donor.

In various embodiments, the system mediates trans-splicing of a pre-mRNA at the 5′ end of the pre-mRNA. In various embodiments, the system mediates trans-splicing of a pre-mRNA at the 3′ end of the pre-mRNA. In various embodiments, the system mediates trans-splicing of a pre-mRNA at an internal site within a pre-mRNA. In various embodiments, the system replaces the 5′ or the 3′ end of the target mRNA.

In various embodiments, the target mRNA is a pre-mRNA. In various embodiments, the system comprises replacing one or more exons of the target mRNA, excluding the first and last exon of the target mRNA. In various embodiments, the nucleic acid binding protein domain mediates trans-splicing of a pre-mRNA within the 3′ untranslated region (UTR). In various embodiments, the nucleic acid binding protein domain mediates trans-splicing of a pre-mRNA within the 5′ untranslated region (UTR).

In various embodiments, the trans-splicing occurs in dividing cells. In various embodiments, the trans-splicing occurs in post-mitotic cells. In various embodiments, the post-mitotic cells are neurons, myocytes, or adipocytes.

In various embodiments, expression of at least one portion of the trans-splicing system is inducible. In various embodiments, activity of at least one portion of the trans-splicing system is inducible. In various embodiments, the nucleic acid binding protein domain is a domain from a zinc finger nuclease (ZFNs) or a Pumby module, or is an RNA recognition motif.

### BRIEF DESCRIPTION OF THE FIGURES

FIG. 1A-1E illustrates the components of the CRISPR/Cas system, cis- and trans-splicing that can occur within a pre-mRNA, and the CRISPR-mediated trans-splicing.

FIG. 2A illustrates the DNA constructs encoding the endoribonuclease-inactive Cas13 (dCas13b)-MS2 fusion protein, the gRNA construct, the splice donor (SD) and splice acceptor (SA) reporters, and the ms2 intron design. FIG. 2B illustrates the trans-splicing reporter assay.

FIG. 3A illustrates the trans-splicing activity of the CRISPR/Cas system compared to negative controls in HEK193FT cells. Columns 1-5 represent activity in the negative controls. Column 11 is the activity of the CRISPR/Cas system. FIG. 3B illustrates the trans-splicing specificity of the CRISPR/Cas system. RNAseq libraries were created using primers that bind to the SA reporter mRNA, and on-target reactions of reads were calculated and plotted.

FIG. 4A is RNAseq mapping of RNAseq libraries to demonstrate trans-splicing. FIG. 4B illustrates validation that there was cis-splicing of the SD reporter.

FIG. 5 is a schematic of gRNA target sites. Several gRNAs were designed to target different regions of the SD reporter.

FIG. 6A illustrates fold-increase in targeted trans-splicing with dCas13b-MS2 as measured by using a truncated GFP trans-splicing reporter assay. FIG. 6B illustrates the targeted trans-splicing without dCas13b-MS2.

FIG. 7 is a schematic that illustrates the components of the CRISPR/Cas system in trans-splicing in the context of correcting a pathogenic exon.

FIG. 8A illustrates induction of the trans-splicing complex with a small molecule. FIG. 8A illustrates a dCas13b polypeptide fused to abscisic acid (ABA)-binding protein ABI1, and MS2 fused to PYL1. FIG. 8B is a graph demonstrating expression of GFP after induction of trans-splicing in the presence of a range of ABA concentrations, shown with a no guide control as a negative control. FIG. 8C demonstrates that ABA could control induced targeted trans-splicing by measuring fold-increase in expression of GFP with increasing concentrations of ABA (mM).

FIG. 9A is a measurement of trans-splicing at splice junctions. FIG. 9A illustrates RNAseq reads spanning possible junctions. FIG. 9B is a plot of spliced reads that were trans-spliced for all transfection conditions. FIG. 9C is a plot of spliced reads that were cis-spliced for all transfection conditions.

FIG. 10A is an illustration of strategies for targeted trans-splicing. FIG. 10A illustrates a strategy for 5′ targeting of trans-splicing. FIG. 10B illustrates an internal targeted trans-splicing strategy. FIG. 10C illustrates a strategy for 3′ targeting of trans-splicing.

FIG. 11A illustrates a strategy for internal exon repair. FIG. 11A illustrates dPspCas13b-MS2, gRNA, target and internal repair template constructs to test if CRISPR-mediated internal exon repair is possible. FIG. 11B illustrates monitoring of expression of the target transcript via expression of blue fluorescent protein (BFP), and monitoring internal exon repair via expression of green fluorescent protein (GFP). FIG. 11C measures GFP expression following internal exon repair, demonstrating that GFP expression is only possible in the presence of the CRISPR system along with gRNAs targeting splice sites.

FIG. 12 is fluorescence measurements to illustrate that trans-splicing can be mediated using other Cas13 orthologs, such as Cas13d.

FIG. 13 demonstrates CRISPR-mediated trans-splicing accomplished with a construct delivered with a multi-vector AAV-based approach.

### DETAILED DESCRIPTION

This disclosure provides compositions and methods of using these compositions to mediate a targeted trans-splicing event on a pre-mRNA in a cell. In various embodiments, the targeted trans-splicing event is mediated by a CRISPR/Cas system. In various embodiments, the CRISPR/Cas system that mediates a targeted trans-splicing event is used to treat a neurodegenerative disease or disorder. These compositions and methods include a trans-splicing event mediated by a CRISPR/Cas system comprising a nuclease-inactive Cas13.

As used within the Claims, the Summary, and the Detailed Description herein, the term “a” or “an” entity refers to one or more of that entity; for example, “a cell” is understood to represent one or more cells.

Furthermore, “and/or” where used herein is to be taken as specific disclosure of each of the two specified features of components with or without the other. Thus, the term “and/or” as used in a phrase such as “A and/or B” herein is intended to include “A and B,” “A or B,” “A” (alone), and “B” (alone).

It is understood that wherever aspects are described herein with the language “comprising,” otherwise analogous aspects described in terms of “consisting of” are also provided.

The term “near a splice site” means within 500 bp of a splice site.

The term “treatment” refers to the application of one or more specific procedures used for the amelioration of a disease. In certain embodiments, the specific procedure is the administration of one or more pharmaceutical agents. “Treatment” of an individual (e.g. a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Treatment includes any desirable effect on the symptoms or pathology of a disease or condition, and may include, for example, minimal changes or improvements in one or more measurable markers of the disease or condition, and may include, for example, minimal changes or improvements in one or more measurable markers of the disease or condition being treated.

The terms “patient,” “subject,” “individual,” and the like are used interchangeably herein, and refer to any animal, or cells thereof whether in vitro or in situ, amenable to the methods described herein. In certain non-limiting embodiments, the patient, subject, or individual is a human.

In aspects of the invention the terms “guide nucleic acid, “RNA guide,” “DNA guide,” and “single guide nucleic acid,” are used interchangeably and refer to the polynucleotide sequence comprising the guide sequence, and the tracr sequence. In certain embodiments, the guide nucleic acid comprises only a crispr RNA (crRNA). The term “guide sequence” refers to a 10-80 bp sequence within the RNA or DNA guide that specifies the target site. In aspects of the invention, the terms “guide sequence” and “spacer” are used interchangeably. In aspects of the invention, the terms “direct repeat” and “scaffold” are used interchangeably.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. In some embodiments, stringent conditions include hybridization in 6× sodium chloride/sodium citrate (SSC) at about 45° C., followed by one or more washes in 0.2×SSC, 0.1% SDS at 50-65° C. Other examples of stringent conditions are known to those skilled in the art and can be found in Current Protocols in Molecular Biology, Ausubel et al., eds., John Wiley & Sons, Inc. (1995), sections 2, 4, and 6, the teachings of which are hereby incorporated by reference herein. Additional stringent conditions can be found in Molecular Cloning: A Laboratory Manual, Sambrook et al., Cold Spring Harbor Press, Cold Spring Harbor, N.Y. (1989), chapters 7, 9, and 11, the teachings of which are hereby incorporated by reference herein.

As used herein, the term “physiological condition” typically refers to biologic parameters that are valid for an animal, specifically a mammal, more specifically a human. The term may relate to biochemical and biophysical parameters commonly found in mammals, especially in the human body (especially body fluids). The “physiological condition” may relate to parameters found in a sick mammal or human patient, as well as that corresponding parameters found in a healthy body. For example, a sick mammal or human patient may have a high but “physiological” temperature condition when the mammal or the human is suffering from a fever. Regarding “physiological conditions” the most important parameters are temperature (37° C. for human body), pH (7.35-7.45 for human blood), osmotic pressure (280-300 mmol/Kg H 2 O) and, if necessary, protein content (66-85 g/l serum). However, those skilled in the art will appreciate that these parameters may vary. For example, such temperature, pH, osmotic pressure, and protein content may be different in a given body or tissue, such as blood or cerebrospinal fluid (Klinke (2005) Physiologie, 5th ed., Georg Thieme Verlag, Stuttgart). “Physiological condition” can also refer to conditions in a buffer system, solvent, and/or excipient that mimic conditions in an animal.

“Hybridization” or “hybridizes” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these.

As used herein, “expression” refers to the process by which a polynucleotide is transcribed from a DNA template (such as into a mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “polypeptide,” “peptide,” “protein,” and “enzyme” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. as used herein the term “amino acid” includes natural and/or unnatural or synthetic amino acids, including glycine and both the D or L optical isomers, and amino acid analogs and peptidomimetics.

The term “nucleic acid” or nucleic acid sequence” refers to a deoxyribonucleic or ribonucleic oligonucleotide in either single- or double-stranded form. The term encompasses nucleic acids, i.e., oligonucleotides, containing known analogues of natural nucleotides. The term also encompasses nucleic-acid-like structures with synthetic backbones, see, e.g., Eckstein, Biomed Biochim Acta. 1991; 50(10-11):S114-7; Baserga et al. Genes Dev. 1992 Jun; 6(6):1120-30; Milligan et al., Nucleic Acids Res. 1993 Jan. 25; 21(2):327-33; WO 97/03211; WO 96/39154; Mata, Toxicol Appl Pharmacol. 1997 May; 144(1):189-97; Strauss-Soukup, Biochemistry. 1997 Aug. 19; 36(33):10026-32; and Samstag, Antisense Nucleic Acid Drug Dev. 1996 Fall; 6(3):153-6.

“Inducible” as used herein refers to inducing expression or activity of a protein or a system, e.g., the trans-splicing system of the instant application. Known inducible gene expression systems have been designed to allow for chemically induced activation of an inserted nucleic acid sequence, resulting in gene overexpression or repression. Inducing activity of a protein or system can include release of a molecule to allow for activity or the addition of an effector molecule to induce activity of a protein or system.

The term “nuclease-inactive” is used to describe a Cas enzyme which no longer has nuclease activity. In some embodiments, a Cas enzyme that no longer has nuclease activity can have a small amount of residual activity. In some embodiments, this small amount of residual activity is less than 5, 1, 0.1, 0.05, 0.01 or 0.005% of wild type nuclease activity of the Cas enzyme. A nuclease-inactive Cas protein may interchangeably be referred to as a “dCas” protein, e.g., dCas13b. In some embodiments, the dCas protein can be a dCas13b protein. In some embodiments, a polynucleotide sequence set forth in SEQ ID NOs: 1, 62, 71, 74, 77, 80, 83, 86, or 89 encodes a dCas13b protein. In some embodiments, dCas13b corresponds to, or comprises in part or in whole, the amino acid sequence set forth as SEQ ID NOs: 2, 63, 72, 75, 78, 81, 84, 87, or 90. In some embodiments, the dCas protein can be a dCas13a protein. In some embodiments, a dCas13a protein is encoded by a polynucleotide sequence set forth in any one of SEQ ID NOs: 17, 20, 23, 26, 29, 32, 35, 38, 41, 44, 47, 50, 53, 56, or 59. In some embodiments, dCas13a corresponds to, or comprises in part or in whole, any one of the amino acid sequence set forth as SEQ ID NOs: 18, 21, 24, 27, 30, 33, 36, 39, 42, 45, 48, 51, 54, 57, or 60. In some embodiments, the dCas protein can be a dCas13d protein. In some embodiments, the dCas13d protein is encoded by the polynucleotide sequence set forth in SEQ ID NO: 65. In some embodiments, the dCas13d protein corresponds to, or comprises in part or in whole, the amino acid sequence set forth in SEQ ID NO: 66. Nuclease-inactive Cas proteins may be variants having mutations which result in nuclease activity inactivated.

“Targeted” is used to describe a molecule, protein, or complex that comprise a targeting moiety which specifically binds to one or more targets associated with a specific pre-mRNA of interest.

As used herein, the term “RNA-binding” is used to describe a molecule, protein, nucleic acid, or complex that specifically binds to RNA.

“Pre-mRNA” refers to a precursor mRNA and is an RNA which contains both exons and intron(s). Pre-mRNA is a type of primary transcript that becomes a messenger RNA after processing. It is synthesized from a DNA template in the cell nucleus by transcription. In some embodiments, RNA is from a mammalian cell. In other embodiments, the RNA is from the mitochondria of a mammalian cell.

The phrase “associates with DNA” refers to nucleic acids, systems, proteins, and molecules that may bind or be in the same vicinity as DNA.

The term “post-mitotic” refers to a non-replicating cell, such as a cell of the nervous system, bone marrow cells, muscle cells, liver cells, and the like. Cells of the nervous system include neurons, glial cells, etc. that is no longer capable of undergoing mitosis.

The term “non-dividing cells” refers to a cell that does not frequently undergo mitosis. Many non-dividing cells may be blocked at any point in the cell cycle (e.g., G0/G1, G1/S, G2/M), as long as most of the cells are not actively dividing. In some embodiments, non-dividing cells are from tissue types that do not frequently divide. Examples of non-dividing cells in the body include, but are not limited to, neuronal, muscle (myocytes), liver, skin, heart, lung, adipose, and bone marrow cells, and their derivatives. “Dividing cells” would be a cell that frequently actively undergoes mitosis. In some embodiments, dividing cells are from tissue types that do not frequently divide. Examples of dividing cells in the body include, but are not limited to, epithelial cells and hematopoietic cells.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health. Diseases and disorders include spinal muscular atrophy, Rett Syndrome, Angelman syndrome, Parkinson's disease, Alzheimer's disease, Huntington's disease, frontotemporal dementia, lysosomal storage diseases, multiple sclerosis, and amyotrophic lateral sclerosis (ALS).

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, herpes simplex virus, anellovirus, baculovirus, retroviral vectors, and the like.

The term “specifically binds,” or the like, means that a given molecule forms a complex with another molecule that is relatively stable under physiologic conditions. Methods for determining whether a given molecule specifically binds to another molecule are well known in the art and include, for example, equilibrium dialysis, surface plasmon resonance, and the like. For example, an RNA hairpin that “specifically binds” a viral protein, as used herein, includes RNA hairpins that bind viral proteins or a portion thereof with a KD of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or about 0.5 nM, as measured in a surface plasmon resonance assay.

“Variant” as used herein means a polypeptide or nucleotide sequence that differs from a given polypeptide or nucleotide sequence in amino acid or nucleic acid sequence by the addition (e.g., insertion), deletion, or conservative substitution of amino acids or nucleotides, but that retains the biological activity of the given polypeptide (e.g., a variant nucleic acid could still encode the same or a similar amino acid sequence). A conservative substitution of an amino acid, i.e., replacing an amino acid with a different amino acid of similar properties (e.g., hydrophilicity and degree and distribution of charged regions) is recognized in the art as typically involving a minor change. These minor changes can be identified, in part, by considering the hydropathic index of amino acids, as understood in the art (see, e.g., Kyte et al., J. Mol. Biol., 157: 105-132 (1982)). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids also can be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity (see, e.g., U.S. Pat. No. 4,554,101). Substitution of amino acids having similar hydrophilicity values can result in peptides retaining biological activity, for example immunogenicity, as is understood in the art. In one aspect, substitutions are performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. “Variant” also can be used to describe a polypeptide or fragment thereof that has been differentially processed, such as by proteolysis, phosphorylation, or other post-translational modification, yet retains its biological activity or antigen reactivity. Use of “variant” herein is intended to encompass fragments of a variant unless otherwise contradicted by context.

Alternatively or additionally, a “variant” is to be understood as a polynucleotide or protein which differs in comparison to the polynucleotide or protein from which it is derived by one or more changes in its length or sequence. The polypeptide or polynucleotide from which a protein or nucleic acid variant is derived is also known as the parent polypeptide or polynucleotide. The term “variant” comprises “fragments” or “derivatives” of the parent molecule. Typically, “fragments” are smaller in length or size than the parent molecule, whilst “derivatives” exhibit one or more differences in their sequence in comparison to the parent molecule. Also encompassed modified molecules such as but not limited to post-translationally modified proteins (e.g. glycosylated, biotinylated, phosphorylated, ubiquitinated, palmitoylated, or proteolytically cleaved proteins) and modified nucleic acids such as methylated DNA. Also mixtures of different molecules such as but not limited to RNA-DNA hybrids, are encompassed by the term “variant”. Typically, a variant is constructed artificially, preferably by gene-technological means whilst the parent polypeptide or polynucleotide is a wild-type protein or polynucleotide. However, also naturally occurring variants are to be understood to be encompassed by the term “variant” as used herein. Further, the variants usable in the present disclosure may also be derived from homologs, orthologs, or paralogs of the parent molecule or from artificially constructed variant, provided that the variant exhibits at least one biological activity of the parent molecule, i.e. is functionally active.

Alternatively, or additionally, a “variant” as used herein, can be characterized by a certain degree of sequence identity to the parent polypeptide or parent polynucleotide from which it is derived. More precisely, a protein variant in the context of the present disclosure exhibits at least 80% sequence identity to its parent polypeptide. A polynucleotide variant in the context of the present disclosure exhibits at least 70% sequence identity to its parent polynucleotide. The term “at least 70% sequence identity” is used throughout the specification with regard to polypeptide and polynucleotide sequence comparisons. This expression preferably refers to a sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference polypeptide or to the respective reference polynucleotide.

The similarity of nucleotide and amino acid sequences, i.e. the percentage of sequence identity, can be determined via sequence alignments. Such alignments can be carried out with several art-known algorithms, preferably with the mathematical algorithm of Karlin and Altschul (Karlin & Altschul (1993) Proc. Natl. Acad. Sci. USA 90: 5873-5877), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80) available e.g. on http://www.ebi.ac.uk/Tools/clustalw/or on http://www.ebi.ac.uk/Tools/clustalw2/index.html or on http://npsa-pbil.ibcp.fr/cgi-bin/npsa_automat.pl?page=/NPSA/npsa_clustalw.html. Preferred parameters used are the default parameters as they are set on http://www.ebi.ac.uk/Tools/clustalw/or http://www.ebi.ac.uk/Tools/clustalw2/index.html. The grade of sequence identity (sequence matching) may be calculated using e.g. BLAST, BLAT or BlastZ (or BlastX). A similar algorithm is incorporated into the BLASTN and BLASTP programs of Altschul et al. (1990) J. Mol. Biol. 215: 403-410. To obtain gapped alignments for comparative purposes, Gapped BLAST is utilized as described in Altschul et al. (1997) Nucleic Acids Res. 25: 3389-3402. When utilizing BLAST and Gapped BLAST programs, the default parameters of the respective programs are used. Sequence matching analysis may be supplemented by established homology mapping techniques like Shuffle-LAGAN (Brudno M., Bioinformatics 2003b, 19 Suppl 1:I54-I62) or Markov random fields. When percentages of sequence identity are referred to in the present application, these percentages are calculated in relation to the full length of the longer sequence, if not specifically indicated otherwise.

### Trans-Splicing

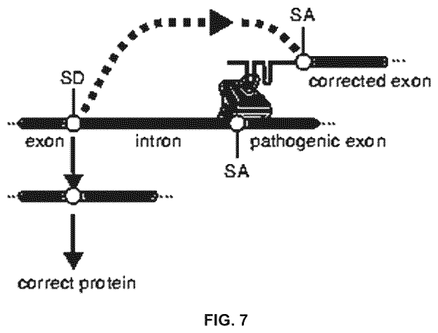

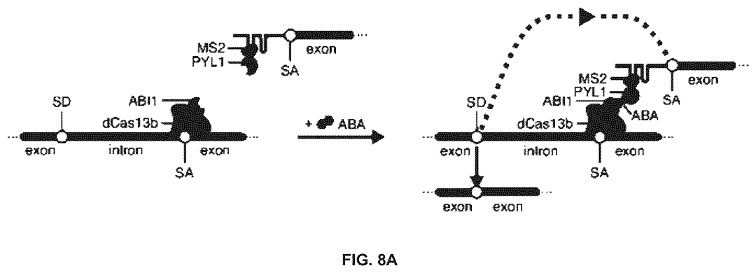

A number of gene therapy techniques have been devised to address specific pathologies. These different approaches range from gene supplementation using viral vectors to genome editing using CRISPR/Cas9 technology. However, there are times when these approaches are not applicable or effective to achieve significant therapeutic effect. Currently, there are efforts to address this problem by repairing mutant transcripts to make clean transcripts without altering gene expression levels by exploiting the spliceosome to catalyze therapeutic trans-splicing events. (See, e.g., FIG. 7).

Splicing is a reaction found in the nucleus of eukaryotic cells catalyzed by the spliceosome, a large ribonucleoproteic complex. Splicing results in the elimination of introns in the pre-mRNA. This reaction is considered cis-splicing as it involves a donor site, a branch point, and an acceptor site located on the same RNA molecule.

The spliceosome can also catalyze trans-splicing events. Unlike cis-splicing, trans-splicing occurs between two different RNA molecules. The molecular process is the same, except that the final mRNA is composed of the first exon(s) of the first pre-mRNA and the exon(s) of another, thus creating a chimeric molecule. With the discovery of naturally occurring trans-splicing, it was demonstrated to be useful for bioengineering purposes. In 1999, Puttaraju et al. (Nature Biotech., 17:246-52) demonstrated the ability of diverting trans-splicing to induce repair of an endogenous mRNA using exon exchange mediated by an artificial RNA capable of inducing trans-splicing in cell culture. Subsequent studies showed the feasibility of using this in vivo, leading to the functional restoration of mutant cystic fibrosis transmembrane conductance regulator (CFTR) in a human bronchial xenograft model system. This led to the study of and use of the spliceosome-mediated RNA trans-splicing, or SMaRT system, as a gene therapy strategy. While this technology has been used to mediate repair of various mRNA sequences associated with disease, there are drawbacks and limitations to the SMaRT technology.

There are some important drawbacks to the current SMaRT technology that one must take into account. One drawback of the SMaRT technology is the specificity of the molecule. Theoretically, off-target trans-splicing with random mRNAs should be processed by nonsense mediated decay or nonstop decay, it is essential to validate the specificity of the pre-mRNA trans-splicing molecule for the target sequence and limit nonspecific events. It has been found that increasing the length of the binding domain sequence up to 153 bases dramatically decreases the probability of finding the entire and exact corresponding sequence in a human genome and increases efficiency. Another drawback is the efficiency of the system. With certain diseases, even a low level of expression of the mutated protein will lead to a diseased phenotype.

### CRISPR/Cas

The present disclosure includes compositions and methods that comprise a targeted trans-splicing system comprising a nuclease-inactive CRISPR/Cas system, a nucleic acid guide, a specific RNA-binding domain, and a repair template comprising an RNA sequences that hybridizes under stringent conditions to the specific RNA-binding domain. Clustered regularly interspaced short palindromic repeats, known more widely as CRISPR, and a family of enzymes known as Cas (CRISPR-associated) proteins is a prokaryotic immune system that confers resistance to foreign genetic elements such as those present within plasmids and phages that provides a form of acquired immunity. Recently, CRISPR/Cas systems have been developed as tools used in basic molecular biology research, the development of biotechnology products, and treatment of disease. These systems have been widely adopted for mediating targeted DNA cleavage which in turn drives targeted gene disruption through non-homologous end joining (NHEJ) or precise gene editing through template-dependent homology-directed repair (HDR).

In certain embodiments, the CRISPR/Cas system described herein comprises a Cas13 enzyme encoded by a polynucleotide sequence of any one of SEQ ID NOs: 1, 17, 20, 23, 26, 29, 32, 35, 38, 41, 44, 47, 50, 53, 56, 59, 62, 65, 71, 74, 77, 80, 83, 86, or 89 or variants thereof. In certain embodiments, the CRISPR/Cas system comprises a Cas13 enzyme comprising an amino acid sequence of any one of SEQ ID NOs: 2, 18, 21, 24, 27, 30, 33, 36, 39, 42, 45, 48, 51, 54, 57, 60, 63, 66, 72, 75, 78, 81, 84, 87, or 90 or variants thereof. Cas13 enzymes are classified as Type VI CRISPR-Cas systems and have two Higher Eukaryotes and Prokaryotes Nucleotide-binding (HEPN) endoRNase domains that mediate precise RNA cleavage with a preference for targets with protospacer flanking site motif observed biochemically and in bacteria. Three Cas13 protein families have been identified to date. Cas13a, previously known as C2c2, can be adapted as tools for nucleic acid detection. Cas13b has been used for both RNA editing and nucleic acid detection, and is unique among the type VI CRISPR effectors in its linear domain architecture and CRISPR RNA (crRNA) structures. Cas13 enzymes are programmable in nature, and makes them an attractive starting point to develop tools for RNA binding and perturbation applications (Cox et al. (2017) Science. 358:1019-1027, incorporated by reference herein in its entirety).

The term “nucleic acid binding protein domain” refers to a domain of a protein that is capable of targeting a specific nucleic acid sequence. In some embodiments, the nucleic acid sequence is DNA. In other embodiments, the nucleic acid sequence is RNA. In some embodiments, the nucleic acid binding protein domain specifically binds to a splice acceptor or a splice donor site. Some examples of nucleic acid binding protein domains include, but are not limited to, zinc finger nucleases (ZFNs), and Pumby modules (Adamala et al. (2016) Proc Natl Acad Sci. 113(19):E2579-E2588, incorporated by reference herein in its entirety), or humanized CRISPR, e.g., CRISPR-Cas-Inspired RNA Targeting System (CIRTS) (Rauch et al. (2019) Cell. 178(1):P122-134, incorporated by reference herein in its entirety), or RNA recognition motif (Maris et al. (2005) The FEBS journal 272: 2118-2131).

The RNA-targeting systems, the vector systems, the vectors and the compositions described herein may be used in various RNA-targeting applications, altering or modifying synthesis of a gene product, such as a protein, RNA cleavage, RNA editing, RNA splicing; trafficking of target RNA, tracing of target RNA, isolation of target RNA, visualization of target RNA, etc.

The term “splice donor” refers to the 5′ end of the intron.

The term “splice acceptor” refers to the 3′ end of the intron.

The term “repair template” refers to a nucleic acid molecule with comprising a desired sequence to be spliced to a nucleic acid locus of interest. In some embodiments, the desired sequence to be spliced is an exon. In some embodiments, the repair template further comprises one or more introns.

In certain embodiments, the nucleic acid guide is a RNA guide. In certain embodiments, the nucleic acid guide is a DNA guide. In some embodiments, the guide nucleic acid, such as a guide RNA or gRNA, is a specific sequence that recognizes the target pre-mRNA of interest and directs the Cas protein to said pre-mRNA. In some embodiments, the guide nucleic acid targets the DNA locus where transcription is occurring. Generally, the guide nucleic acid is made of two components—a crispr RNA (crRNA), which is a 17-20 nucleotide sequence complementary to the target sequences, and a tracr RNA, which serves as a binding scaffold for the Cas nuclease. More recently, the crRNA and tracr RNA components have been fused into one molecule to create a single guide RNA (sgRNA). In certain embodiments, the trans-splicing system comprises one nucleic acid guides. For currently characterized CRISPR/Cas13 systems, a crispr RNA (crRNA) is comprised of a spacer and a direct repeat. For Cas13b, the spacer is 5′ and the direct repeat is the 3′ of the crRNA. For Cas13b, crRNA and guide RNA can be used interchangeably. For Cas13a and Cas13d, the spacer is 3′ and the direct repeat is the 5′ of the crRNA (Zhang F (2019). Development of CRISPR-Cas systems for genome editing and beyond. Quarterly Reviews of Biophysics 52, e6, 1-31. https://doi.org/10.1017/S0033583519000052, incorporated by reference herein in its entirety). In certain embodiments, the trans-splicing system comprises more than one nucleic acid guide. In certain embodiments, more than one nucleic acid guides are in an array operably linked to one promoter, which are then cleaved and processed by Cas13. In some embodiments, an array has direct repeats between multiple spacers wherein each spacer targets a different nucleic acid. In certain embodiments, more than one nucleic acid guides are expressed by separate promoters, such as U6 promotors. In certain embodiments, the guide RNAs have a direct repeat (e.g., SEQ ID NOs: 10, 19, 22, 25, 28, 31, 34, 37, 40, 43, 46, 49, 52, 55, 58, 61, 64, 67, 73, 76, 79, 82, 85, 88, and 91) at the 5′ end, and a spacer at the 3′ end. In other embodiments, the guide RNAs have a direct repeat (e.g., SEQ ID NOs: 10, 19, 22, 25, 28, 31, 34, 37, 40, 43, 46, 49, 52, 55, 58, 61, 64, 67, 73, 76, 79, 82, 85, 88, and 91) at the 3′ end, and a spacer at the 5′ end. In other embodiments, spacers are flanked by direct repeats (e.g., SEQ ID NOs: 10, 19, 22, 25, 28, 31, 34, 37, 40, 43, 46, 49, 52, 55, 58, 61, 64, 67, 73, 76, 79, 82, 85, 88, and 91). In certain embodiments, the direct repeats have at least 70% (e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%) sequence identity with the direct repeat nucleic acid sequences of SEQ ID NOs: 10, 19, 22, 25, 28, 31, 34, 37, 40, 43, 46, 49, 52, 55, 58, 61, 64, 67, 73, 76, 79, 82, 85, 88, or 91. In certain embodiments, the nucleic acid guides recognize multiple targets. In certain embodiments, the instant disclosure provides nucleic acid guide sequences having at least 70% (e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%) sequence identity with the nucleic acid sequence of SEQ ID NO: 11 or 12.

The invention uses nucleic acids to bind target RNA sequences. This is advantageous as nucleic acids are much easier and cheaper to produce than proteins, and the specificity can be varied according to the length of the stretch where homology is sought. Complex 3-D positioning of multiple fingers, for example is not required. The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. The term also encompasses nucleic-acid-like structures with synthetic backbones, see, e.g., Eckstein, 1991; Baserga et al., 1992; Milligan, 1993; WO 97/03211; WO 96/39154; Mata, 1997; Strauss-Soukup, 1997; and Samstag, 1996. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

In certain embodiments, the specific RNA binding domain of the CRISPR/Cas system comprises a viral protein fused to the Cas protein. The term “viral protein” can be used to describe a protein of viral origin that is bound to the Cas protein that binds to a hairpin of the splice acceptor template. Coat proteins of single-stranded RNA bacteriophages are translational repressors of viral replicase. They accomplish this by specifically binding an RNA hairpin that encompasses the replicase start codon. Some examples would be the coat proteins of RNA phages MS2, λN, QBeta, and PP7 (e.g. SEQ ID NOs: 5-9). In certain embodiments, the specific RNA binding domain comprises an amino acid sequence of any one of SEQ ID NOs: 6, 7, 8, or 9, or variants thereof. The term “binding”, or the like, means that a viral protein forms a complex with a corresponding hairpin that is relatively stable under physiologic conditions. In certain embodiments, the instant disclosure provides hairpin sequences having at least 70% (e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%) sequence identity with any one of the nucleic acid sequences of SEQ ID NOs: 13-16.

In certain embodiments, the viral protein is covalently bound to the CRISPR/Cas system. In certain embodiments, the viral protein is not covalently bound to the CRISPR/Cas system. In certain embodiments, the viral protein and CRISPR/Cas system is a fusion protein. As used herein, the term “covalently bound” refers to a chemical bond that involves the sharing of electrons between atoms. The term “fusion protein” refers to proteins created through the joining of two or more genes that originally coded for separate proteins.

In some embodiments, the specific RNA binding domain of the CRISPR/Cas system comprises an RNA binding protein that is not of viral origin.

In certain embodiments, the trans-splicing system comprises one repair template. In certain embodiments, the trans-splicing system comprises more than one repair template.

In certain embodiments, the trans-splicing system comprises a nucleic acid guide-binding protein domain comprising a nuclease-inactive CRISPR/Cas system.

In certain embodiments, expression and/or the activity of the trans-splicing system is transient. “Transient expression” refers to the temporary expression of genes that are expressed for a time after a nucleic acid has been introduced into eukaryotic cells. In some embodiments, transient expression of the trans-splicing system may be controlled with a small molecule. As used herein, the term “small molecule” refers to a non-nucleic acid/non-amino acid molecule. As used herein, the term “small molecule binding domain” refers to a portion of a molecule, often a protein, that specifically binds to a given small molecule. These can include a tet-ON or tet-OFF system or chemogetic control using synthetic transcription factors and protease inhibitors. For example, dCas13-NLS-NS3-MS2 remains active and uncleaved in the presence of BILN-2061. (Tague et al., Nature Methods, Volume 15, pages 519-522 (2018) and Wagner et al. Nature Chemical Biology, Volume 14, pages 1043-1050 (2018), incorporated by reference herein in their entireties). In some embodiments, transient expression of the trans-splicing system can be controlled by degradation of the delivery particle. In some embodiments, expression of the trans-splicing system may be controlled with a light-activated transcription factor (Konermann et al., Nature, Volume 500, pages 472-476 (22 Aug. 2013), incorporated by reference herein in its entirety). In some embodiments, assembly of the trans-splicing system may be controlled with a small molecule, e.g., chemically induced dimerization. Examples include, dCas13-NLS-FKBP and FKBP-NLS-MS2, assembled by FK1012; dCas13-NLS-FKBP and CNA-NLS-MS2, assembled by FK506; and dCas13-NLS-FRB and MS2-FKBP transiently assembled by rapamycin, and rapidly disassembled by FK506. (Braun et al., Nature Communications, DOI: 10.1038/s41467-017-00644-y, incorporated by reference herein in its entirety). Additional examples include dCas13-NLS-ABI1 and PYL1-MS2, assembled by abscisic acid (Gao et al., Nature Methods, DOI: https://doi.org/10.1038/nmeth.4042, incorporated by reference herein in its entirety). Other examples of small molecules and their chemically induced systems are included in Stanton et al. (Science, DOI: http://dx.doi.org/10.1126/science.aao5902, incorporated by reference herein in its entirety). In some embodiments, activity of the trans-splicing system can be controlled using a small molecule to degrade the system. In some embodiments, the small molecule binding domain may be linked to the CRISPR system via a self-cleaving peptide. In some embodiments, the self-cleaving peptide is a 2A self-cleaving peptide. Some examples include, but are not limited to, T2A, P2A, E2A, and F2A sequences.

In some embodiments, assembly of the trans-splicing system is mediated by a SunTag (Tanenbaum et al., Cell, 2014, DOI: https://doi.org/10.1016/j.cell.2014.09.039). In some embodiments, assembly of the trans-splicing system is mediated by hairpins on the guide RNA, either native or modified, such as the SAM system (Konermann et al., Nature, 2015, DOI: 10.1038/nature14136).

In some embodiments, transient expression is performed by delivering the trans-splicing system as RNA as shown in Hewitt et al., Science Translational Medicine 30 Jan. 2019: Vol. 11, Issue 477, eaat9143, incorporated by reference herein in its entirety. In some embodiments, the trans-splicing system assembly can be controlled via light. For example, dCas13-NLS-CRY2 and CIB1-NSL-MS2 are assembled by 466 nm light. (Konermann et al. (2013)). In some embodiments, transient expression is performed by delivering the trans-splicing system with an episomal or non-integrating virus. These viruses include Ad5, AAV, HSV-1, or baculovirus. In some embodiments, activity of the trans-splicing system is mediated by conditionally active inteins, such as inteins that undergo protein splicing in the presence of 4-hydroxytamoxifen (4-HT) or other small molecules. In some embodiments, activity of the trans-splicing system is controlled by 4-HT by including a 4-HT sensitive intein into Cas13 at a location that disrupts Cas13 activity until 4-HT mediated protein splicing has taken place, similar to Davis et al. (Nature Chemical Biology, 2015, DOI: https://doi.org/10.1038/nchembio.1793).

### Delivery

In some embodiments, the nucleic acid introduced into the eukaryotic cell is a plasmid DNA or viral vector.

Preferably, delivery is in the form of a vector which may be a viral vectors, such as a lenti- or baculo- or adeno-viral/adeno-associated viral vectors, but other means of delivery are known (such as yeast systems, microvesicles, gene guns/means of attaching vectors to gold nanoparticles) and are provided. The viral vector may be selected from a variety of families/genera of viruses, including, but not limited to Myoviridac, Siphoviridae, Podoviridae, Corticoviridae, Lipothrixviridae, Poxviridae, Tridoviridae, Adenoviridae, Polyomaviridae, Papillonaviridae, Mimiviridae, Pandoravirusa, Salterprovirusa, Inoviridae, Microviridae, Parvoviridae, Circoviridae, Hepadnaviridae, Caulimoviridae, Retroviridae, Cystoviridae, Reoviridae, Birnaviridae, Totiviridae, Partitiviridae, Filoviridae, Orthomyxoviridae, Deltavirusa, Leviviridae, Picornaviridae, Marnaviridae, Secoviridae, Potyviridae, Caliciviridae, Hepeviridae, Astroviridac, Nodaviridae, Tetraviridac, Luteoviridae, Tombusviridae, Coronaviridae, Arteriviridae, Flaviviridae, Togaviridae, Virgaviridae, Bronoviridae, Tymoviridae, Alphaflexiviridae, Sobemovirusa, or Idaeovirusa.

A vector may mean not only a viral or yeast system (for instance, where the nucleic acids of interest may be operably linked to and under the control of (in terms of expression, such as to ultimately provide a processed RNA) a promoter), but also direct delivery of nucleic acids into a host cell. While in herein methods the vector may be a viral vector and this is advantageously AAV, other viral vectors as herein discussed can be employed, such as lentivirus. For example, baculoviruses may be used for expression in insect cells. These insect cells may, in turn be useful for producing large quantities of further vectors, such as AAV or lentivirus adapted for delivery of the present invention. Also envisaged is a method of delivering the present CRISPR enzyme comprising delivering to a cell mRNA encoding the CRISPR enzyme. It will be appreciated that in certain embodiments the CRISPR enzyme is truncated, and/or comprised of less than one thousand amino acids or less than four thousand amino acids, and/or is a nuclease or nickase, and/or is codon-optimized, and/or comprises one or more mutations, and/or comprises a chimeric CRISPR enzyme, and/or the other options as herein discussed.

In some embodiments, expression of a nucleic acid sequence encoding a CRISPR enzyme may be driven by a promoter. In some embodiments, a single promoter drives expression of a nucleic acid sequence encoding a CRISPR enzyme and one or more of the guide sequences. In some embodiments, the CRISPR enzyme and guide sequence(s) are operable linked to and expressed from the same promoter. In some embodiments, the CRISPR enzyme and guide sequence(s) are expressed from different promoters. For example, the promoter(s) can be, but are not limited to, a UBC promoter, a PGK promoter, an EF1A promoter, a CMV promoter, an EFS promoter, a SV40 promoter, and a TRE promoter. The promoter may be a weak or a strong promoter. The promoter may be a constitutive promoter or an inducible promoter. In some embodiments, the promoter can also be an AAV ITR, and can be advantageous for eliminating the need for an additional promoter element, which can take up space in the vector. The additional space freed up by use of an AAV ITR can be used to drive the expression of additional elements, such as guide sequences. In some embodiments, the promoter may be a tissue specific promoter.

In some embodiments, an enzyme coding sequence encoding a CRISPR enzyme is codon-optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “codon usage tabulated from the international DNA sequence databases: status for the year 2000” Nucl. Acids Res, 28:292 (2000). Computer algorithms for codon optimizing a particular sequence for expression in a particular host cell are also available, such as Gene Forge (Aptagen; Jacobus, Pa.), are also available. In some embodiments, one or more codons (e.g., 1, 2, 3, 4, 5, 10, 15, 20, 25, 50, or more, or all codons) in a sequence encoding a Cas protein correspond to the most frequently used codon for a particular amino acid.

In some embodiments, a vector encodes a Cas protein comprising one or more nuclear localization sequences (NLSs), such as about or more than about 1, 2, 3, 4, 5, 6, 7, 8, 9, 10, or more NLSs. In some embodiments, the Cas protein comprises about or more than 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., one or more NLS at the amino-terminus and one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. Typically, an NLS consists of one or more short sequences of positively charged lysines or arginines exposed on the protein surface, bur other types of NLS are known. In some embodiments, the NLS is between two domains, for example between the Cas13 protein and the viral protein. The NLS may also be between two functional domains separated or flanked by a glycine-serine linker.

In general, the one or more NLSs are of sufficient strength to drive accumulation of the Cas protein in a detectable amount in the nucleus of a eukaryotic cell. In general, strength of nuclear localization activity may derive from the number of NLSs in the Cas protein, the particular NLS used, or a combination of these factors. Detection of accumulation in the nucleus may be performed by any suitable technique. For example, a detectable marker may be fused to the Cas protein, such that location within a cell may be visualized, such as in combination with a means for detecting the location of the nucleus (e.g., a stain specific for the nucleus such as DAPI). Examples of detectable markers include fluorescent proteins (such as green fluorescent proteins, or GFP; RFP; CFP), and epitope tags (HA tag, FLAG tag, SNAP tag). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of CRISPR complex formation (e.g. assay for DNA cleavage or mutation at the target sequence, or assay for altered gene expression activity affected by CRISPR complex formation and/or CRISPR complex activity), as compared to a control not exposed to the CRISPR complex, or exposed to a Cas protein lacking the one or more NLSs.

In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner). ClustalW, ClustalX, BLAT, Novoalign (Novocraft Technologies, ELAND (Illumina, San Diego, Calif.), SOAP (available at soap.genomics.org.cn), and Mag (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of a CRISPR complex to a target sequence may be assessed by any suitable assay.

In some aspects, the invention provides methods comprising delivering one or more polynucleotides, such as one or more vectors as described herein, one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, a Cas protein in combination with (and optionally complexed) with a guide sequence is delivered to a cell. Conventional viral and non-viral based gene transfer methods can be used to introduce nucleic acids in mammalian cells or target tissues. Such methods can be used to administer nucleic acids encoding components of a CRISPR system to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-8313 (1992); Navel and Feigner, TIBTECH 11:211-217 (1993); Mitani and Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer and Perricaudet, British Medical Bulletin 51(1):31-44 (1995); Haddada et al., in Current Topics in Microbiology and Immunology, Doerfler and Bohm (eds) (1995); and Yu et al., Gene Therapy 1:13-26 (1994).

The CRISPR/Cas system as described herein, can be delivered using adeno-associated virus (AAV), lentivirus, adenovirus, or other viral vector types, or combinations thereof. Cas protein(s) and one or more guide RNAs can be packaged into one or more viral vectors. In some embodiments, the targeted trans-splicing system is delivered via AAV as a split intein system, similar to Levy et al (Nature Biomedical Engineering, 2020, DOI: https://doi.org/10.1038/s41551-019-0501-5). In other embodiments, the targeted trans-splicing system can be delivered via AAV as a trans-splicing system, similar to Lai et al. (Nature Biotechnology, 2005, DOI: 10.1038/nbt1153). In some embodiments, the viral vector is delivered to the tissue of interest by, for example, an intramuscular injection, while other times the viral delivery is via intravenous, transdermal, intranasal, oral, mucosal, intrathecal, intracranial or other delivery methods. Such delivery may be either via a single dose, or multiple doses. One skilled in the art understands that the actual dosage to be delivered herein may vary greatly depending upon a variety of factors, such as the vector chosen, the target cell, organism, or tissue, the general condition of the subject to be treated, the degree of transformation/modification sought, the administration route, the administration mode, the type of transformation/modification sought, etc.

The use of RNA or DNA viral based systems for the delivery of nucleic acids takes advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues. Viral-mediated in vivo delivery of Cas13 and guide RNA provides a rapid and powerful technology for achieving precise miRNA perturbations within cells, especially in post-mitotic cells and tissues.

In certain embodiments, delivery of the trans-splicing system to a cell is viral. In certain embodiments, the virus is a retrovirus, adenovirus, adeno-associated virus, herpes simplex virus, anellovirus, or baculovirus.

In certain embodiments, delivery of the trans-splicing system to a cell is non-viral. In certain embodiments, the non-viral delivery system is selected from a cationic lipid vehicle, electroporation, calcium phosphate transfection, transfection through membrane disruption using mechanical shear forces, mechanical transfection, and nanoparticle delivery.

Preferably, the vector is a viral vector, such as a lenti-, baculo-, or adeno-viral/adeno-associated viral vectors, but other means of delivery are known (such as yeast systems, microvesicles, gene guns/means of attaching vectors to gold nanoparticles) and are provided. In some embodiments, one or more of the viral or plasmid vectors may be delivered via liposomes, nanoparticles, exosomes, microvesicles, or a gene-gun.

Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

In another embodiment, minimal non-primate lentiviral vectors based on the equine infectious anemia virus (EIAV) are also contemplated, especially for ocular gene therapy (see, e.g., Balagaan, J gene Med 2006: 8:275-285, Published online 21 Nov. 2005 in Wiley InterScience (www.interscience.wiley.com). DOI: 10.1002/jgmn.845). In another embodiment, RetinoStat, an equine infectious anemia virus-based lentiviral gene therapy vector that expresses angiostatic proteins endostatin and angiostatin that is delivered via a subretinal injection for the treatment of the web form of age-related macular degeneration is also contemplated (see, e.g., Binley et al., HUMAN GENE THERAPY 23:980-991 (September 2012)) may be modified for the CRISPR-Cas system of the present invention.

Lentiviral vectors have been disclosed as in the treatment for Parkinson's Disease, see, e.g., US Patent Publication No. 20120295960 and U.S. Pat. Nos. 7,303,910 and 7,351,585. Lentiviral vectors have also been disclosed for delivery to the brain, see, e.g., US Patent Publication Nos. US20110293571, US20040013648; US20070025970, US20090111106, and U.S. Pat. No. 7,259,015.

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386; 4,946,787; and 4,897,355; and lipofection reagents are sold commercially (e.g., Transfectam™ and Lipofectin™). Cationic and neutral lipids that are suitable for efficient receptor-recognition lipofection of polynucleotides include those of Felgner, WO 91/17424: WO 91/16024. Delivery can be to cells (e.g., in vitro or ex vivo administration) or target tissues (e.g., in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-289 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183; 4,217,344; 4,235,871; 4,261,975; 4,485,054; 4,501,728; 4,774,085; 4,837,028; and 4,946,787).

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. Cell lines are available from a variety of sources known to those with skill in the art (see, e.g., the American Type Culture Collection (ATCC) (Manassas, VA)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

In some embodiments, one or more vectors described herein are used to produce a non-human transgenic animal or transgenic plant. In some embodiments, the transgenic animal is a mammal, such as a mouse, rate, or rabbit. In certain embodiments, the organism or subject is a plant. In certain embodiments, the organism or subject or plant is algae. Methods for producing transgenic plants and animals are known in the art, and generally begin with a method of cell transfection, such as described herein.

In one aspect, the invention provides for methods of modifying a target polynucleotide in a eukaryotic cell. In some embodiments, the method comprises allowing a CRISPR complex to bind to the target polynucleotide to effect cleavage of said target polynucleotide thereby modifying the target polynucleotide, wherein the CRISPR complex comprises a CRISPR enzyme complexed with a guide sequence hybridized to a target sequence within said target polynucleotide, wherein said guide sequence is linked to a tracr mate sequence which in turn hybridizes to a tracr sequence.

With recent advances in crop genomics, the ability to use CRISPR/Cas systems to perform efficient and cost effective gene editing and manipulation will allow the rapid selection and comparison of single and multiplexed genetic manipulations to transform such genomes for improved production and enhanced traits. In this regard, reference is made to US patents and publications: U.S. Pat. No. 6,603,061—Agrobacterium-Mediated Plant Transformation Method; U.S. Pat. No. 7,868,149—Plant Genome Sequences and Uses Thereof and US 2009/0100536—Transgenic Plants with Enhanced Agronomic Traits, all the contents and disclosure of each of which are herein incorporated by reference in their entirety. In the practice of the invention, the contents and disclosure of Morell et al “Crop genomics: advances and application” Nat Rev Genet. 2011 Dec. 29; 13(2):85-96 are also herein incorporated by reference in their entirety.

Any components of the instant CRISPR/Cas system can be delivered in the form of RNA. Cas and/or viral protein mRNA can be generated using in vitro transcription. For example, Cas mRNA can be synthesized using a PCR cassette containing the following elements: T7 promoter-Kozak sequence (GCCACC)-Cas13-3′ UTR from beta globin-polyA tail (a string of 120 or more adenines). The cassette can be used for transcription by T7 polymerase. Guide RNAs can also be transcribe using in vitro transcription from a cassette containing T7 promoter-CC-guide RNA sequence.

The components of the instant CRISPR/Cas system may be delivered simultaneously using nanoparticles or lipid envelopes. For example, Su X, Fricke J, Kavanaugh D G. Irvine D J (“In vitro and in vivo mRNA delivery using lipid-enveloped pH-responsive polymer nanoparticles” Mole Phar. 2011 Jun. 6; 8(3):774-87. doi: 10.1021/mp100390w. Epub 2011 Apr 1) describes biodegradable core-shell structured nanoparticles with a poly(fi-amino ester) (PBAE) core enveloped by a phospholipid bilayer shell. These were developed for in vivo mRNA delivery. The pH-responsive PBAE component was chosen to promote endosome disruption, while the lipid surface layer was selected to minimize toxicity of the polycation core. Such are, therefore, preferred for delivering RNA of the present invention.

In one embodiment, nanoparticles based on self-assembling bioadhesive polymers are contemplated, which may be applied to oral delivery of peptides, intravenous delivery of peptides and nasal delivery of peptides, all to the brain. Other embodiments, such as oral absorption and ocular deliver of hydrophobic drugs are also contemplated. The molecular envelope technology involves an engineered polymer envelope which is protected and delivered to the site of the disease (see, e.g., Mazza, M. et al. ACSNano, 2013. 7(2):1016-1026; Siew, A., et al. Mol Pharm, 2012. 9(1):14-28; Lalatsa, A., et al. J Contr Rel, 2012. 161(2):523-36; Lalatsa, A., et al., Mol Pharm, 2012. 9(6):1665-80; Lalatsa, A., et al. Mol Phar, 2012. 9(6):1764-74; Garrett, N. L., et al. J. Biophotonics, 2012. 5(56):458-68; Garrett, N. L, et al. J Rainan Spect, 2012. 43(5):681-688; Ahmad, S., et al. J Royal Soc Interface 2010. 7:5423-33; Uchegbu, LF. Expert Opin Drug Deliv. 2006. 3(5):629-40; Qu, X., et al. Biomacromolecules, 2006. 7(12):3452-9 and Uchegbu, I. F., et al. Int J Pharm, 2001. 224:185-199) Doses of about 5 mg/kg are contemplated, with single or multiple doses, depending on the target tissue.

US Patent Application 20110293703 relates to lipidoid compounds are also particularly useful in the administration of polynucleotides, which may be applied to deliver the CRISPR/Cas system of the present invention. In one aspect, the aminoalcohol lipidoid compounds are combined with an agent to be delivered to a cell or a subject to form microparticles, nanoparticles, liposomes, or micelles. The agent to be delivered by the particles, liposomes, or micelles may be in the form of a gas, liquid, or solid, and the agent may be a polynucleotide, protein, peptide, or small molecule. The aminoalcohol lipidoid compounds may be combined with other aminoalcohol lipidoid compounds, polymers (synthetic or natural), surfactants, cholesterol, carbohydrates, proteins, lipids, etc. to form the particles. These particles may then optionally be combined with a pharmaceutical excipient to form a pharmaceutical composition.

US Patent Application 20130302401 relates to a class of poly(beta-amino alcohols) (PBAAs) has been prepared using combinatorial polymerization. The inventive PBAAs may be used in biotechnology and biomedical applications as coatings (such as coatings of films or multilayer films for medical devices or implants), additives, materials, excipients, non-biofueling agents, micropatterning agents, and cellular encapsulation agents. When used as surface coatings, these PBAAs elicited different levels of inflammation, both in vitro and in vivo, depending on their chemical structures. The large chemical diversity of this class of materials allowed us to identify polymer coatings that inhibit macrophage activation in vitro. Furthermore, these coatings reduce the recruitment of inflammatory cells, and reduce fibrosis, following the subcutaneous implantation of carboxylated polystyrene microparticles. These polymers may be used to form polyelectrolyte complex capsules for cell encapsulation. The invention may also have many other biological applications such as antimicrobial coatings, DNA or siRNA delivery, and stein cell tissue engineering. The teachings of US Patent Application No. 20130302401 may be applied to the CRISPR/Cas system of the present invention.

In another embodiment, lipid nanoparticles (LNPs) are contemplated. In particular, an antitransthyretin small interfering RNA encapsulated in lipid nanoparticles (see, e.g., Coelho et al., N Engl J Med 2013; 369:819-29) may be applied to the CRISPR/Cas system of the present invention.

The charge of the LNP must be taken into consideration. As cationic lipids combined with negatively charged lipids to induce nonbilayer structures that facilitate intracellular delivery. Because charged LNPs are rapidly cleared from circulation following intravenous injection, ionizable cationic lipids with pKa values below 7 were developed (see, e.g., Rosin et al, Molecular therapy, vol. 19, no. 12, pages 1286-2200, Dec. 2011). Negatively charged polymers such as siRNA oligonucleotides may be loaded into LNPs at low pH values (e.g., pH 4) where the ionizable lipids display a positive charge. However, at physiological pH values, the LNPs exhibit a low surface charge compatible with longer circulation times. Four species of ionizable cationic lipids have been focused upon, namely 1,2-dilineoyl-3-dimethylammonium-propane (DLinDAP), 1,2-dilinoleyoxy-3-N,N-dimethylaminopropane (DLinDMA), 1,2-dilinoleyloxy-keto-N,N-dimethyl-3-aminopropane (DLinKDMA), and 1,2-dilinoleyl-4-(2-dimethylaminoethyl)-[1,3]-dioxolane (DLinKC2-DMA).

Self-assembling nanoparticles with siRNA may be constructed with polyethyleneimine (PEI) that is PEGylated with an Arg-Gly-Asp (RGD) peptide ligand attached at the distal end of the polyethylene glycol (PEG), for example, as a means to target tumor neovasculature expressing integrins and used to deliver siRNA inhibiting vascular endothelial growth factor receptor-2 (VEGFR2) expression and thereby tumor angiogenesis (see, e.g., Schiffelers et al., Nucleic Acids Research, 2004, Vol. 32. No. 19). Nanoplexes may be prepared by mixing equal volumes of aqueous solutions of cationic polymer and nucleic acid to give a net molar excess of ionizable nitrogen (polymer) to phosphate (nucleic acid) over the range of 2 to 6. The electrostatic interactions between cationic polymers and nucleic acid resulted in the formation of polyplexes with average particle size distribution of about 100 nm, hence referred to her as nanoplexes.

Exosomes are endogenous nano-vesicles that transport RNAs and proteins which can deliver short interfering (si)RNA to the brain in mice. To reduce immunogenicity, Alvarez-Erviti et al. (2011, Nat Biotechnol 29: 341) used self-derived dendritic cells for exosome production.

In certain embodiments, the invention provides a method of treating or inhibiting a condition caused by a defect in a target sequence in a mRNA of interest in a subject (e.g., mammal or human) or a non-human subject (e.g., mammal) in need thereof comprising modifying the subject or a non-human subject by manipulation of the target sequence and wherein the condition is susceptible to treatment or inhibition by manipulation of the target sequence comprising providing treatment comprising: delivering a non-naturally occurring or engineered composition comprising an AAV or lentivirus vector system comprising one or more AAV or lentivirus vectors operably encoding a composition for expression thereof, wherein the target sequence is manipulated by the composition when expressed, wherein the composition comprises: (A) a non-naturally occurring or engineered composition comprising a vector system comprising one or more vectors comprising a targeted trans-splicing system comprising a non-natural nucleic acid guide-binding protein domain, a nucleic acid guide, a specific RNA-binding domain, and a repair template comprising a splice donor and/or splice acceptor and an RNA sequence that hybridizes under stringent conditions to the specific RNA-binding domain.

### Targeting of Cells

The work herein supports the use of CRISPR/Cas systems to target pre-mRNA in post-mitotic cells through delivery of the CRISPR/Cas system to the appropriate location (i.e., to cells within the organs or tissues of interest) to mediate trans-splicing events to pre-mRNA.

In certain embodiments, the CRISPR/Cas system further comprises a cell. In certain embodiments, the CRISPR/Cas system is targeted to RNA. In certain embodiments, the CRISPR/Cas system is associated with RNA in the cell. In certain embodiments, the RNA is a pre-mRNA. In certain embodiments, the CRISPR/Cas system mediates trans-splicing of pre-mRNA at intron-exon junctions. In certain embodiments, the CRISPR/Cas system mediates trans-splicing of pre-mRNA at exon-intron junctions. In certain embodiments, the CRISPR/Cas system mediates trans-splicing of a pre-mRNA at the 3′ end of the pre-mRNA. Examples of a sequence targeting the 3′ end of a pre-mRNA may include, but is not limited to SEQ ID NO: 70, as disclosed herein. In certain embodiments, the CRISPR/Cas system mediates trans-splicing of pre-mRNA within the 3′ untranslated region (UTR). Trans-splicing within the 3′ UTR can lead to stronger translation of the resulting mRNA. In certain embodiments, the CRISPR/Cas system mediates trans-splicing of a pre-mRNA at an internal site within a pre-mRNA. Examples of a sequence targeting an internal site within a pre-mRNA may include, but is not limited to SEQ ID NO: 92, as disclosed herein. In various embodiments, the CRISPR/Cas system mediates trans-splicing of pre-mRNA within the 5′ untranslated region (UTR). The 3′ and 5′ UTRs of mRNA are known to contain multiple regulatory elements and are critical for the stability and translation of mRNA into protein. Warren et al. (Cell Stem Cell, Volume 7, pages 618-630 (2010), incorporated by reference herein in its entirety) used an artificial 5′ UTR containing a strong Kozak translation signal and the alpha globin 3′ UTR to improve protein production during reprogramming of fibroblasts to induced pluripotent stem cells. In certain embodiments, the CRISPR/Cas system can mediate trans-splicing to provide a 3′UTR sequence to change the strength of translation, in some cases leading to stronger translation and significant increases in protein production. For example, 3′ UTRs from genes including, but are not limited to, AGXT, ALB, APOA2, ASL, C3, CYP2E1, FBA, HPX, and/or ORM, can be used to change the strength of translation. Also, for example, 5′ UTRs from genes including, but are not limited to, AGXT, ALB, APOA2, ASL, C3, CYP2E1, FBA, HPX, and/or ORM, can be used to change the strength of translation.

In certain embodiments, the CRISPR/Cas system is associated with DNA in the cell. In certain embodiments, the CRISPR/Cas system is not associated with DNA in the cell.

In certain embodiments, the trans-splicing system is introduced into the cell. In certain embodiments, the CRISPR/Cas system mediates trans-splicing of pre-mRNA in the nucleus of a cell. In certain embodiments, the CRISPR/Cas system mediates trans-splicing of pre-mRNA in the cytoplasm of a cell. In another aspect of the invention, provided herein is a method of mediating a targeted trans-splicing event on a pre-mRNA in a cell, the method comprising introducing a targeted trans-splicing CRISPR/Cas system into the cell, wherein the system comprises a nuclease-inactive nucleic acid-targeting CRISPR/Cas system, a nucleic acid guide that specifically hybridizes to a nucleic acid locus of interest, a specific RNA-binding domain, and a repair template comprising an RNA sequence that hybridizes under stringent conditions to the specific-RNA-binding domain.

Gene editing using Type II, and more recently Type V, CRISPR systems can be accomplished through either of two pathways: non-homologous end joining (NHEJ) or homology-directed repair (HDR). NHEJ does not require the cells to be actively dividing, however HDR is only active in dividing cells. In certain embodiments, the CRISPR/Cas system mediates trans-splicing of pre-mRNA in the nucleus of the cell. In certain embodiments, the CRISPR/Cas system mediates trans-splicing of pre-mRNA in the cytoplasm of the cell. The cell can be a dividing cell, or a post-mitotic cell. In certain embodiments, the post-mitotic cell can be simply one or post-mitotic cells, or an organ per se or a tissue within it. In certain embodiments, the post-mitotic cell can be selected from the group consisting of a neuron, myocyte, and adipocyte. The post-mitotic cells may be comprised within a vertebrate animal, either a patient (in the sense of an animal in need of trans-splicing mediated treatment) or a model organism, or may be in cell culture, an organoid, or other ex vivo tissue, such a “liver on a chip” for instance where hepatocytes are seeded and grown on a scaffold. With the development of 3-D printing techniques being applied to biology, printed tissues are within grasp and it is entirely feasible that liver cells or tissues printed I this way to create an organoid or onto a chip could also be targeted. Non-liver alternatives are also envisaged, particularly for other post-mitotic cells/tissues.

Thus, provided is a model organism comprising post-mitotic cells, such as neurons or kidney cells, to which the present CRISPR-Cas system has been delivered. Such collections may include post-mitotic organs, organoids, or cells populating a scaffold (‘kidney on a chip’).

In particular, such post-mitotic cells may express, or comprise polynucleotides capable of expressing, a Cas enzyme. As discussed herein, this has the advantage of providing a ready model for interrogating gene product function through targeted-trans-splicing of pre-mRNA. This is particularly useful in studying conditions of the post-mitotic cells, such as the kidney or brain, such as those listed herein, as well as broader conditions such as obesity.

Also provided is a method of inducing transcript perturbation in one or more post-mitotic cells, comprising transducing population of cells with a CRISPR/Cas system according to the present invention to thereby alter transcripts of a population of cells. The method may be ex vivo or in vitro, for instance in a cell culture or in an ex vivo or in vitro model (such as an organoid). Alternatively, the method may be in vivo, in which case it may also include isolating a population of cells from the subject, and transplanting the population of cells (back) into the subject. Transcript perturbation may be for one or more, or two or more, or three or more, or four or more genes. However, if the cells already comprise Cas, whether expressed as a protein or encoded by polynucleotides already comprised within the cells, then only the CRISPR polynucleotide needs to be delivered. The method may include extraction from and, optionally, re-insertion back into the post-mitotic cell. By delivering, it is meant actually physical delivery of the polynucleotides to the nucleus of the cell, but also transfection. Therefore, delivery should also be read as including transfection unless otherwise apparent.

### Gene Therapy

Because the described invention can be used to mediate trans-splicing in a non-dividing cell, in certain embodiments the cell is in a subject suffering from a disease or disorder. In certain embodiments the disease or disorder is a neurodegenerative, neurological, or neuromuscular disease or disorder. In certain embodiments, the disease or disorder is selected from the group consisting of spinal muscular atrophy, Rett Syndrome, Angelman syndrome, Parkinson's disease, Alzheimer's disease, Huntington's disease, frontotemporal dementia, lysosomal storage diseases, multiple sclerosis, and amyotrophic lateral sclerosis (ALS).

The approaches taken herein demonstrate that the instant invention can be applied to gene therapy. For instance, correction of one or more deficient genotypes (for example single point mutations) is achievable through the use of the present CRISPR-Cas system in the post-mitotic cells discussed herein. Monogenic conditions associated with the post-mitotic are particularly preferred and are exemplified herein.

Although one guide may be used, so-called multiplexing with two, three, four or more guides, is particularly useful in gene therapy where multiple defective genotypes are to be corrected (either multiple errors in a single gene or multiple errors spread across several genes.

Accordingly, in certain embodiments the invention provides a method of modifying post-mitotic cells of an organism, e.g., mammal including a human or a non-human mammal or organism by manipulation of a target sequence in a genomic locus of interest comprising delivering a non-naturally occurring or engineered composition comprising a viral or plasmid vector system comprising one or more viral or plasmid vectors operably encoding a composition for expression thereof, wherein the composition comprises: (A) a non-naturally occurring or engineered composition comprising a vector system comprising one or more vectors comprising a targeted trans-splicing system comprising a non-natural nucleic acid guide-binding protein domain, a nucleic acid guide, a specific RNA-binding domain, and a repair template comprising a splice donor and/or splice acceptor and an RNA sequence that hybridizes under stringent conditions to the specific RNA-binding domain.

The trans-splicing system as described herein can be used to interrogate the function of one or more genes in post-mitotic cells. This may be achieved through delivery and expression of the CRISPR/Cas system to the post-mitotic cell, wherein the guide(s) of the CRISPR/Cas system are designed to recruit the CRISPR/Cas system to the pre-mRNA target or targets of interest. Equally, where the CRISPR/Cas is already comprised within the post-mitotic cell, protein (transcribed) form, then delivery of the guides to the post-mitotic cell will suffice. Having a CRISPR/Cas system induced by a small molecule may be advantageous here. Where the CRISPR/Cas is already within the post-mitotic cell, in polynucleotide (untranscribed), then delivery of the guides to the post-mitotic cell as well as induction of transcription of the Cas9 polynucleotide will be necessary. Having the CRISPR/Cas system under the control of an inducible or repressible promoter, such as the tet (tetracycline) on-off system may be advantageous here.

One aspect that is particularly promising is the integration of CRISPR techniques with phenotypic assays to determine the phenotypic changes, if any, resulting from gene perturbations, especially knock downs. Use of the CRISPR/Cas system can be combined with biochemical, sequencing, electrophysiological, and behavioral analysis to study the function of the targeted genomic element.

This, in one aspect, there is provided a method of interrogating the function of one or more genes in a post-mitotic cell, comprising inducing an expression of a modulated mRNA and determining changes in phenotype due to one or more genes in the condition thereby interrogating the function of the one or more proteins translated from the modulated pre-mRNA.

The following applies broadly to appropriate aspects of the invention. The cell may be in a subject, such as a human, animal, or model organism, so that protein function is interrogated in vivo, however, it is also envisaged that the cell may be ex vivo, for instance in a cell culture or in a model organ or organoid. In some embodiments, the method may include isolation of a first population of cells from the subject, optionally culturing them and transducing them with one or more CRISPR/Cas systems. Further optional culturing may follow. Transplantation of the transduced cells back into the subject may then occur.

The cell may be from any of the tissues or organism described herein. The brain is one preferred example, providing for said method of interrogating the function of one or more gene products, such as pre-mRNA, wherein the post-mitotic cell is a brain cell, for instance a neuron. Particularly in vivo, this allows for the interrogation of mutated or modified protein function on animal behavior. The animal is preferably a mammal, for instance a rodent. Given the complexity of the nervous system, which consists of intricate networks of heterogeneous cell types, being able to efficiently edit pre-mRNAs of neurons in vivo enables direct testing of gene function in relevant cell types embedded in native contexts.

### Kits

The present disclosure provides kits for carrying out a method. In one aspect, the invention provides kits containing any one or more of the elements disclosed in the above methods and compositions. In some embodiments, the kit comprises a vector system and instructions for using the kit. In some embodiments, the kit comprises a vector system comprising regulatory elements and polynucleotides encoding the CRISPR/Cas trans-splicing system. In some embodiments, the kit comprises a viral delivery system of the CRISPR/Cas trans-splicing system. In some embodiments, the kit comprises a non-viral delivery system of the CRISPR/Cas trans-splicing system. Elements may be provided individually or in combinations, and may be provided in any suitable container, such as a vial, a bottle, or a tube. In some embodiments, the kit includes instruction in one or more languages, for examples, in more than one language.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a guide sequence for insertion into a vector so as to operably link the guide sequence and a regulatory element.

The invention is further described in detail by reference to the following experimental examples. These examples are included merely for purposes of illustration of certain aspects and embodiments of the present invention, and are not intended to limit the invention. Thus, the invention should in no way be construed as being limited to the following examples, but rather, should be construed to encompass any and all variation which become evident as a result of the teaching provided herein.

### EXAMPLES

### Example 1—Engineering of the CRISPR-Based Trans-Splicing System

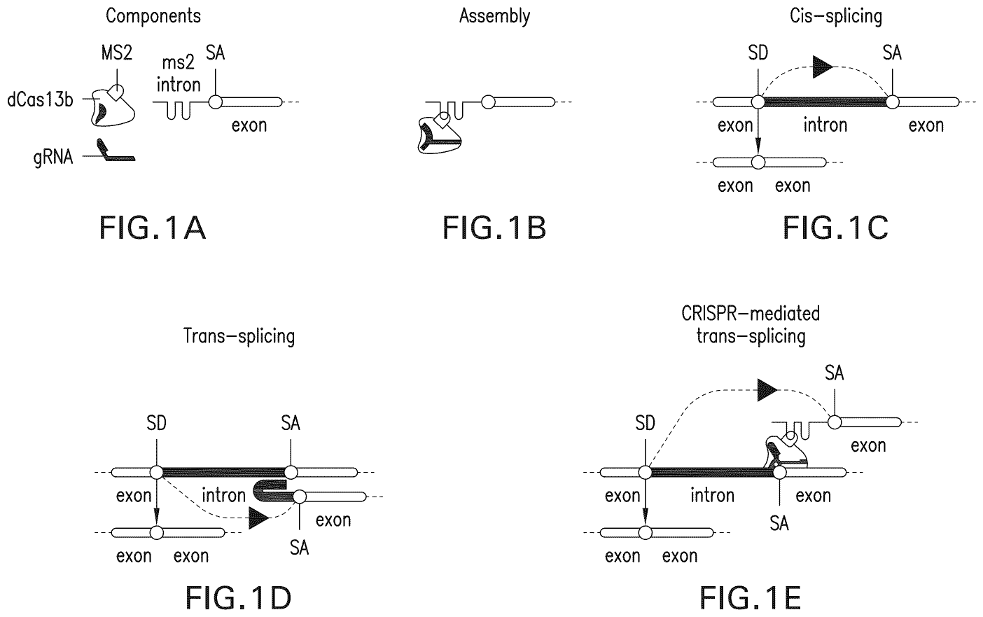

A CRISPR-based trans-splicing system was engineered to comprise a guide RNA, such as a guide RNA (gRNA), an exon or a group of exons of interest conjugated to an engineered intron with ms2 hairpins, and a catalytically inactive Cas13b (dCas13b) linked to a MS2 binding protein (FIG. 1A). Assembly of the trans-splicing system relies on the dCas13b recognizing the gRNA scaffold, and the MS2 binding protein recognizing the ms2 hairpins of the engineered intron (FIG. 1B). Instead of inducing a cis-splicing reaction (FIG. 1C), the CRISPR-dCas13b system induces a trans-splicing event similar to the endogenous spliceosome-based reaction that is illustrated in FIG. 1D. In the CRISPR/Cas-based system, the trans-splicing event occurs when the above described CRISPR-dCas13b assembly binds to a pre-mRNA. When the dCas13b that is linked to a MS2 binding protein binds to the pre-mRNA, the cis-splice acceptor is blocked. The MS2 binding protein tethers a trans-splicing RNA molecule of interest, and with the binding of the dCas13b to the target pre-mRNA, directs the trans-splicing RNA to the pre-mRNA. This enables highly efficient trans-splicing (FIG. 1E).

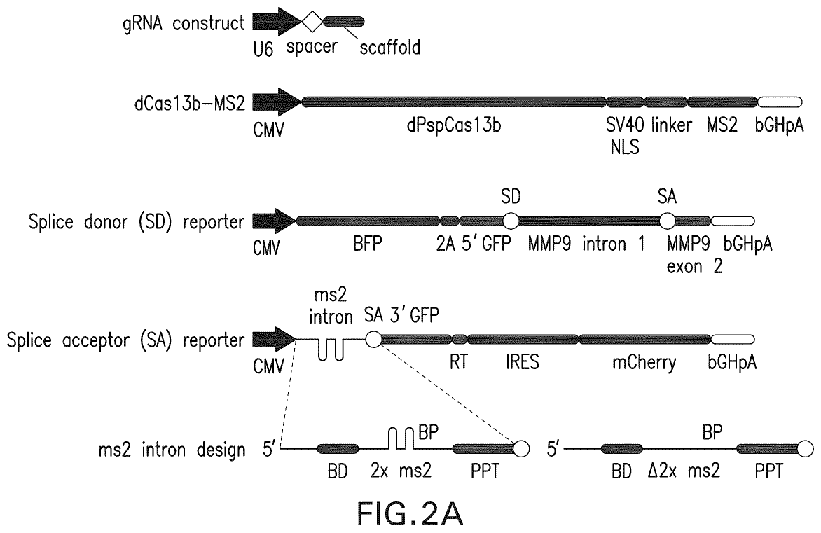

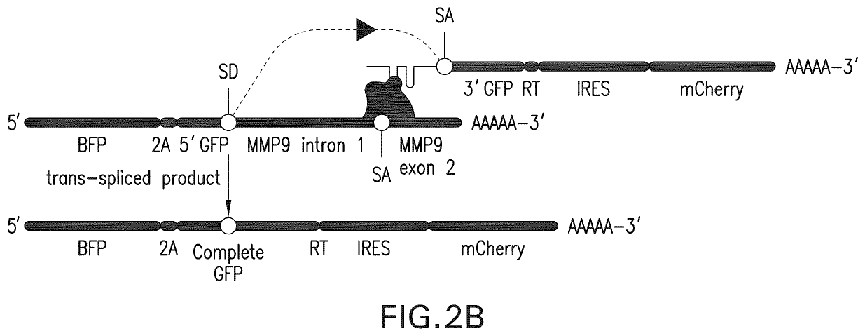

Several DNA constructs were utilized to reduce CRISPR-mediated trans-splicing to practice (FIG. 2A). First, a U6 promoter-driven gRNA construct with a Prevotella sp. Cas13b (PspCas13b) RNA scaffold was used to drive expression of the gRNA in the cells. The dCas13b was linked to a MS2 binding protein via a SV40 nuclear localization signal (SV40 NLS) and glycine-serine linker. Bovine growth hormone polyadenylation signal (bGHpA) was used to allow for stable expression of the construct. A splice donor was used to generate expression of a pre-mRNA that could be assayed for trans-splicing. Blue fluorescent protein (BFP) was used as a marker to validate expression of the splice donor, and a self-cleaving p2A linker was used on truncated GFP (5′ GFP). To ensure the splice acceptor would undergo trans-splicing, Matrix Metallopeptidase 9 (MMP9) intron 1 and exon 2 were placed downstream, followed by bGHpA to ensure stable expression. A splice acceptor reporter was designed, such that trans-splicing would generate a complete GFP, which could be observed by flow cytometry. The designed ms2 intron consists of i) a binding domain (BD) which hybridizes to MMP9 intron 1 in the splice donor reporter, ii) two ms2 loops, iii) a branch point (BP), and iv) a polypyrimidine tract (PPT), followed by v) a splice acceptor sequence. The second half of the truncated GFP was placed behind the ms2 intron, which was then followed by a reverse transcription primer binding site (RT), allowing for generation of next generation sequencing libraries (NGS) to validate trans-splicing via RNAseq. To confirm expression of the splice acceptor reporter, an internal ribosome entry site (IRES) was used to drive translation of mCherry, thus allowing mCherry measurement via flow cytometry independent of trans-splicing. Lastly, a ms2 null (Δ2×ms2) splice acceptor reporter was used to measure the effect of ms2 loops on CRISPR-mediated trans-splicing efficiency.

Upon successful trans-splicing, the truncated GFP exons (5′ GFP, 3′ GFP) introduced by the splice donor and acceptor reporters are spliced together, generating a transcript that contains a complete GFP mRNA sequence capable of creating a functional GFP protein product (FIG. 2B). Cells were gated for BFP+ and mCherry+using flow cytometry, to validate expression of both the splice donor (SD) and splice acceptor (SA) reporters respectively. From the gated double positive BFP+mCherry+population, trans-splicing activity was measured by the fraction of cells that were GFP+.

### Example 2—Measuring CRISPR-Mediated Trans-Splicing

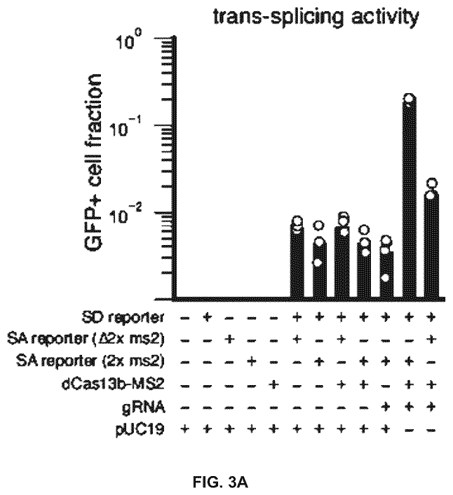

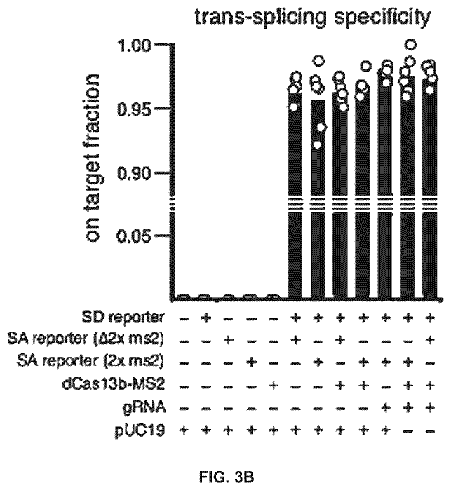

HEK293FT cells were transfected in a 12-well plate format, with a total of 1250 ng of DNA and 4 μL of Lipofectamine 2000 per condition. Each construct was one fourth of the total DNA transfection, with the exception of pUC19, which was used as non-coding control DNA in conditions where less than four components were delivered. A total of 1250 ng of DNA was delivered. Media was changed 6 hours post-transfection, and the cells were analyzed via flow cytometry 48 hours after transfection. Negative controls (columns 1-5 of FIG. 3A) showed no detection of trans-splicing via the GFP reporter assay. Column 6, which represents the state-of-the-art for trans-splicing, led to detection of trans-splicing in 0.69%±0.08% (mean±SD, n=4) of BFP+mCherry+ cells. CRISPR-mediated trans-splicing (column 11) led to detection of trans-splicing in 19.58%±1.01% (mean±SD, n=4) of BFP+mCherry+ cells, which was a 28.03±1.45-fold increase. As expected, CRISPR-mediated trans-splicing had a significant decrease in efficiency when utilizing the ms2 null (Δ2×ms2) SA reporter (column 12). Interestingly, there was a significant increase in trans-splicing compared to the case where dCas13b-MS2 and a gRNA were not expressed (column 6) by 2.45±0.45-fold (p=0.00074), suggesting that binding dCas13b to the SA prevents cis-splicing, thereby promoting trans-splicing in contexts where there are no further exons for cis-splicing.

RNAseq libraries were created by carrying out reverse transcription on the SA reporter using a reverse transcription primer that binds to the RT site on the SA reporter mRNA. The RT site was designed to also carry a TruSeq adapter sequence, such that read 1 of the final library would be the 3′ end of GFP. The read 2 sequencing adapter was randomly inserted via tagmentation. Reads were filtered by having read 1 match the 3′ end of GFP. Read 2 was then aligned using BWA to a whole transcriptome reference concatenated with the SD and SA reporters. The on-target fraction (plotted) was calculated by dividing the number of reads that map to the SD reporter by the number of total reads that align to the transcriptome (including the SD reporter) but do not align to the SA reporter (FIG. 3B).

### Example 3—Generation of RNAseq Library to Measure On-Target Trans-Splicing

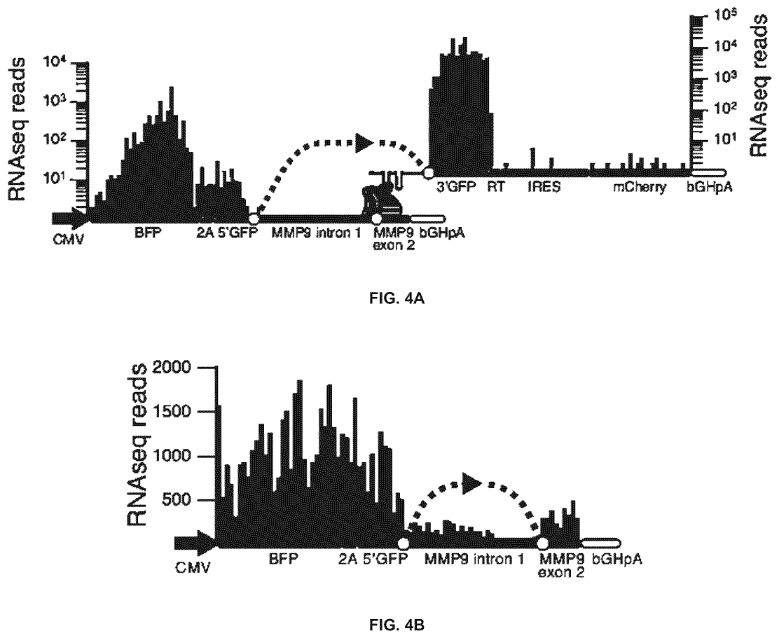

RNAseq libraries generated by RT of the SA reporter were analyzed for mapping position of read 2. As expected, read 2 showed high mapping to the exonic region of the SD reporter, demonstrating trans-splicing. In contrast, only 1 read mapped to the MMP9 intron and MMP9 exon 2, which was likely from index-hopping or chimeric events (FIG. 4A).

To validate cis-splicing of the SD reporter, RNAseq libraries were generated by reverse transcription of the poly(A) tail. As expected, cis-splicing was observed as the number of reads was lower for the intronic region (MMP intron 1) and higher for the exonic regions (BFP-2A-5′GFP and MMP9 exon 2) (FIG. 4B).

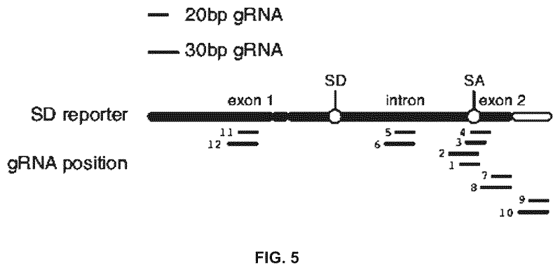

To better understand the gRNA design constraints for CRISPR-mediated trans-splicing, several gRNAS were designed to target different regions of the SD reporter, gRNAS-1-4 targeted the SA site, while gRNAs 5 and 6 targeted the intron. gRNAs 7 and 8 targeted MMP9 exon 2, while gRNAs 9 and 10 targeted bGHpA. gRNAs 11 and 12 targeted BFP in the first exon (FIG. 5).

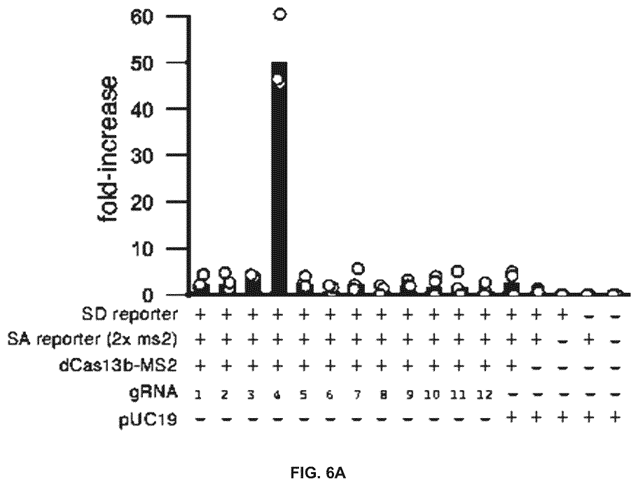

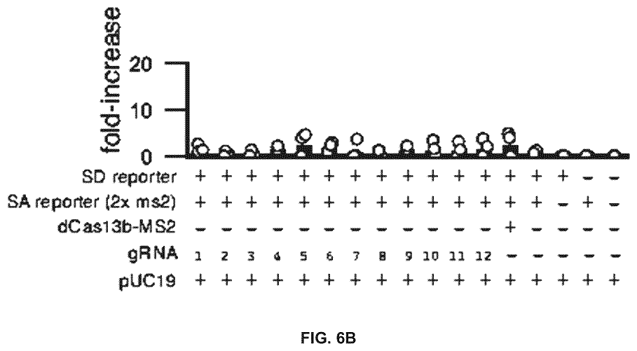

Targeted trans-splicing was measured by using the truncated GFP trans-splicing reporter assay. Flow cytometry was conducted on transfected cells with 3 biological replicates per condition. When using a gRNA that targets the splice acceptor site, CRISPR-mediated trans-splicing improved trans-splicing efficiency by ˜50-fold with a gRNA designed to target the splice acceptor site. In this case, the most effective gRNA (gRNA 4) has 3′ guide mapping to the splice acceptor site. As expected, targeting the CRISPR system to alternative regions of the SD reporter result in marginal gains in trans-splicing efficiency (FIG. 6A).

Fold-increases in targeted trans-splicing are marginal when excluding dCas13b-MS2, as measured by the truncated GFP trans-splicing reporter assay (FIG. 6B). Flow cytometry was conducted on transfected cells with three biological replicates per condition.

### Example 4—Trans-Splicing Induced by a Small Molecule

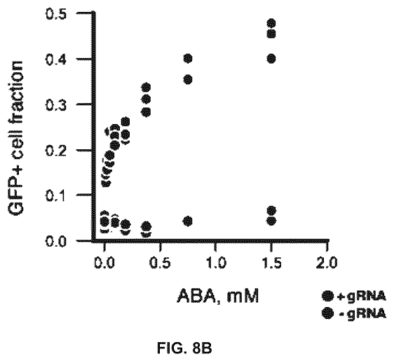

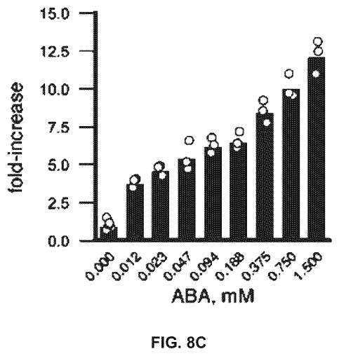

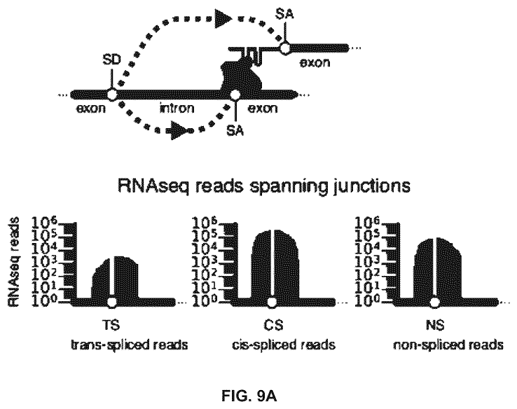

In order to test if the trans-splicing dCas13b complex could be modified to induce trans-splicing with a small molecule, dCas13b was fused to ABI1, and the MS2 was fused to PYL1, both via glycine-serine linkers (FIG. 8A). It was hypothesized that with this architecture, trans-splicing can be transiently induced via introduction of abscisic acid (ABA), as the machinery assembles in the presence of ABA. The construct delivered by a single expression cassette was a CMV-dPspCas13b-ABI1-2A-PYL1-MS2. A split GFP reporter was utilized to assay trans-splicing activity via GFP measurement through flow cytometry.

Using the construct as described above, the split GFP reporter was trans-spliced in the presence of 1.5 mM ABA, resulting in >40% of cells being GFP+(FIG. 8B). HEK293FT cells were transfected using Lipofectamine 2000 per the manufacturer's instructions, and flow cytometry was conducted 72 hours after transfection. ABA was titrated across multiple samples, and 3 biological replicates per ABA concentration were measured via flow cytometry. To demonstrate that ABA could control the induced targeted trans-splicing, a split GFP reporter was trans-spliced in the presence of ABA, and the fold-change in GFP translation was tightly controlled by ABA concentration (FIG. 8C).

### Example 5—Measuring Trans-Splicing at Splice Junctions

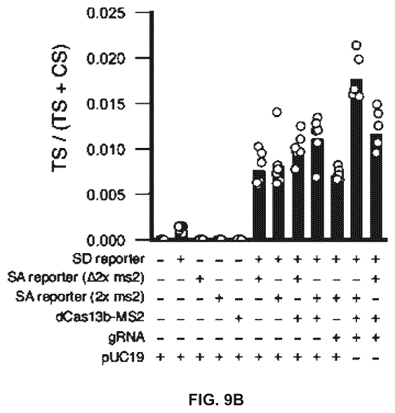

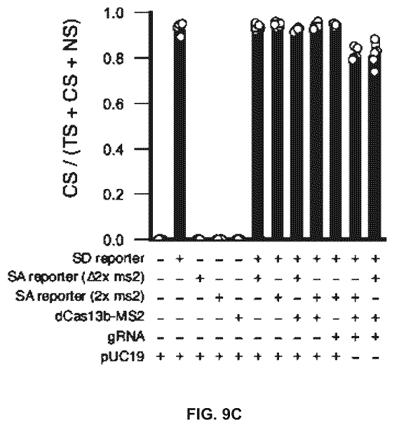

To measure exon junctions, full-length RNAseq was conducted on 293FT cells transfected with dCas13b-MS2, gRNA, and the split GFP reporter system (SD reporter, SA reporter with 2×ms2). Cells were gated for BFP+ and mCherry+ via flow cytometry, to validate expression of both the splice donor (SD) and splice acceptor (SA) reporters respectively, and were then sorted into TCL buffer to lyse the cells prior to RNAseq library construction. RNAseq reads spanning all possible junctions were filtered using regular expressions, and were subsequently mapped onto all possible junction sequences, namely, trans-spliced junctions (TS), cis-spliced junctions (CS), and non-spliced junctions (NS). The mapping positions were the plotted to illustrate reads spanning possible junctions (FIG. 9A).

Full length RNAseq reads spanning all possible junctions were filtered using regular expressions, and were subsequently mapped onto all possible junction sequences, namely, the trans-spliced junction (TS), cis-spliced junction (CS), and non-spliced junction (NS). A fraction of spliced reads that were trans-spliced were calculated and plotted for all transfection conditions (FIG. 9B). Full length RNAseq reads spanning all possible junctions were filtered using regular expressions, and were subsequently mapped onto all possible junction sequences, namely the TS, CS, and NS as above. Fractions of the spliced reads that were cis-spliced was calculated and plotted for all transfection conditions (FIG. 9C). Results how that the reporter undergoes efficient splicing, and that dCas13 inhibits cis-splicing in the presence of a targeting gRNA.

### Example 6—Strategies for Targeted Trans-Splicing

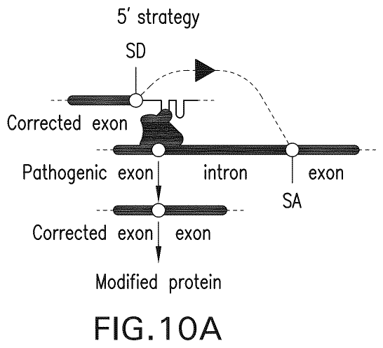

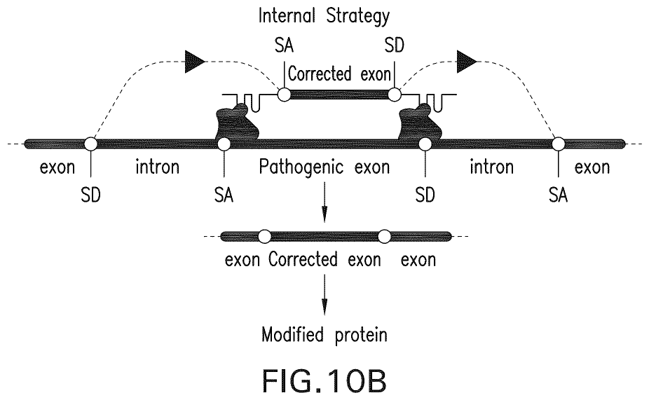

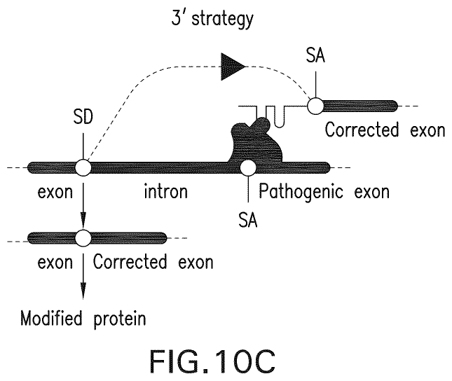

FIG. 10A illustrates a strategy for 5′ targeting of trans-splicing. For this strategy, a dCas13b-MS2 or analogue, can be targeted to a splice donor (SD), while simultaneously providing a 5′ repair template for 5′ correction or modification of RNA. Such a strategy may be advantageous when the correction or modification is near the 5′ end of the mRNA. FIG. 10B illustrates an internal targeted trans-splicing strategy. A dCas13b-MS2 or analogue, can be targeted both to a splice acceptor, and a splice donor, while simultaneously providing a repair template with an exon, or group of exons, possessing both a splice acceptor and splice donor. FIG. 10C illustrates a strategy for 3′ targeting of trans-splicing. For this strategy, a 3′ repair template may be provided for 3′ correction or modification of RNA, along with a dCas13b-MS2, or analogue, targeting a splice acceptor (SA). Such a strategy may be advantageous when the correction or modification is near the 3′ end of the mRNA.

### Example 7—Internal Exon Repair

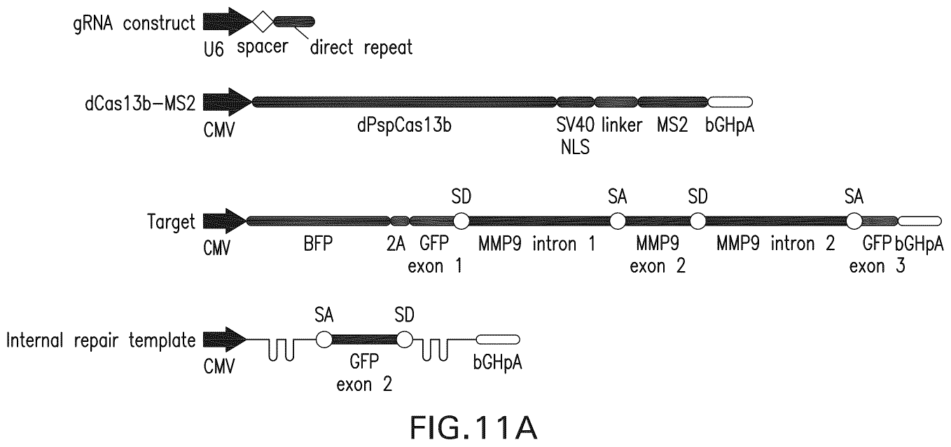

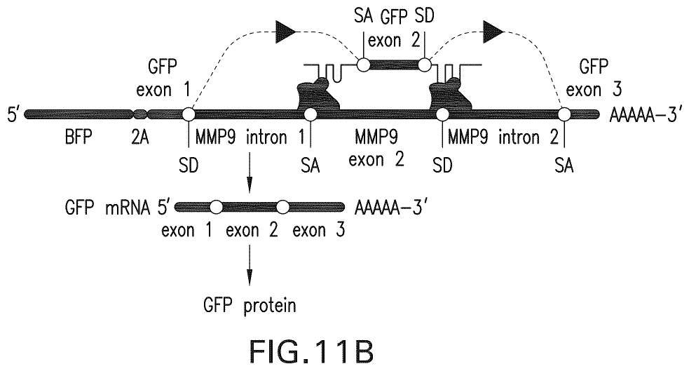

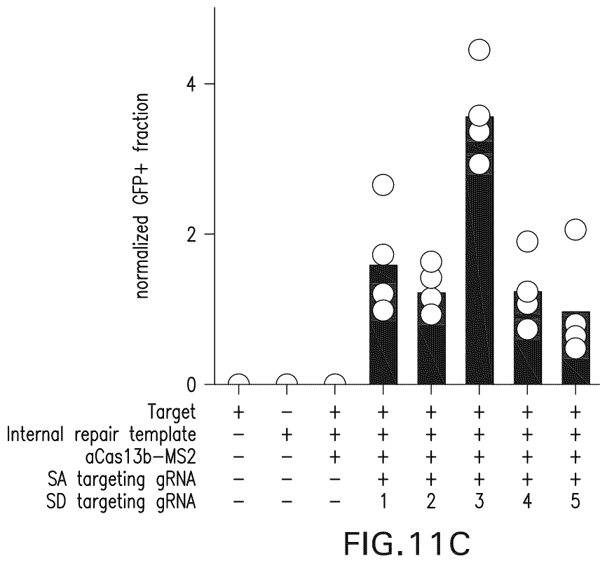

FIG. 11A illustrates a construct to be used for internal exon repair. Standard gRNA constructs were used, along with CMV-dPspCas13b-MS2 to test whether internal exon repair is possible with CRISPR-mediated trans-splicing. GFP was split into three exons, and GFP exons 1 and 3 were utilized in the target RNA molecule design. To simulate a pathogenic exon, MMP9 exon 2 was placed between GFP exons 1 and 3 on the target molecule, along with corresponding flanking introns: MMP9 intron 1 and MMP9 intron 2. The internal repair template was designed to have GFP exon 2 flanked by two synthetic introns, each with an ms2 hairpin.

FIG. 11B illustrates an assay for monitoring internal exon repair. The target transcript is monitored via expression of blue fluorescent protein (BFP). Cells expressing the target transcript are GFP negative, as the middle exon of GFP (GFP exon 2) is missing from the transcript. Upon internal exon repair, GFP exon 2 from the repair template is trans-spliced in order to create a complete GFP mRNA, and the BFP moiety is lost via the T2A self cleaving peptide. The presence of GFP mRNA leads to translation and expression of GFP protein, which can be measured through flow cytometry.

FIG. 11C is a readout of GFP expression following internal exon repair. 293FT cells were transfected using lipofectamine 2000 per manufacturer's instructions and cells were transfected in a 96 well format (100 ng per well) with four biological replicates for each condition. Flow cytometry was conducted 48 hours after transfection. Several gRNAs targeting the splice donor (SD) were designed (SD targeting gRNAs 1-5), and delivered in conjunction with a splice acceptor (SA) targeting gRNA. In the absence of gRNAs, no GFP+ cells were detected. However, upon introduction of gRNAs, GFP+ cells were detected and the fraction of GFP+ cells varied depending on the placement of the gRNA relative to the splice donor.

### Example 8—Use of Cas13 Orthologs

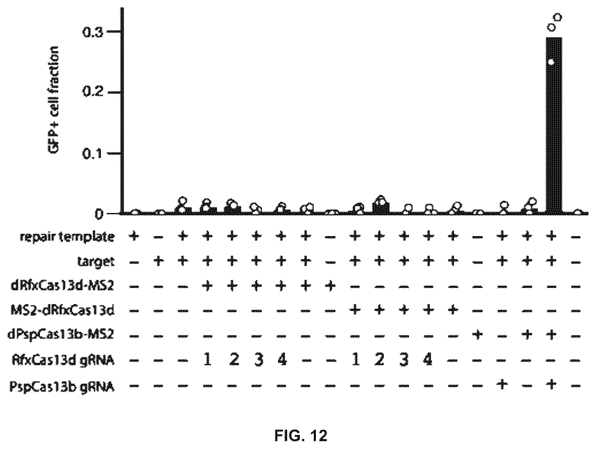

To test whether Cas13d could also lead to CRISPR-mediated trans-splicing, dRfxCas13d was benchmarked against dPspCas13b. Four guides were designed for RfxCas13d, which all targeted the splice acceptor site in the target reporter. MS2 was fused to the N and C-terminus in two different Cas13d architectures. 293FT cells were transfected using lipofectamine 2000 per manufacturer's instructions and cells were transfected in a 96 well format (100 ng per well) with three biological replicates per condition. Flow cytometry was conducted 48 hours after transfection, and cells were gated on BFP and mCherry in order to only consider cells with both target and repair template reporters. BFP+mCherry+ cells were gated on GFP to determine trans-splicing frequency. Mild trans-splicing was observed in the RfxCas13d systems, which were far outperformed by the PspCas13b system (FIG. 12).

### Example 9—AAV-Based Delivery of CRISPR Trans-Splicing System

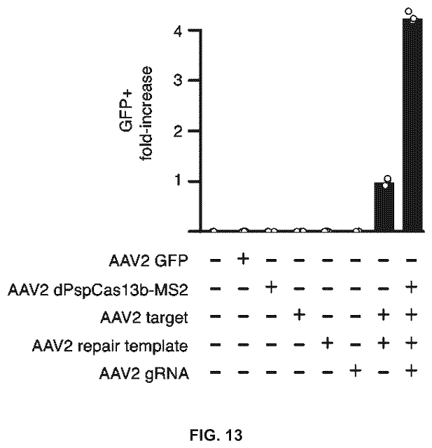

To test whether the CRISPR system could be delivered with a multi-vector AAV-based approach, individual components were cloned into constructs with AAV2 ITRs to enable AAV packaging. AAV2/8 was produced in 293FT cells by PEI co-transfecting each transfer vector with Rep2/Cap8 (pAAV2/8) and AAV helper plasmid (pAdDeltaF6) in a 3:5:6 ratio at 30 μg of DNA per 150 mm dish. Media was changed 24 hours after transfection, and supernatants were harvested 72 hours after transfection. Supernatant was then filtered with a 0.45 um cellulose acetate filter and AAV was concentrated by using a 100 kDa MWCO amicon filter at 4000 ref for 30 minutes at 4 C. 293FT cells were then transduced in suspension using 3 uL of AAV per well in a 96 well plate with four biological replicates per condition. Flow cytometry was conducted 72 hours after AAV transduction in order to measure CRISPR-mediated trans-splicing. Cells were gated on BFP and mCherry in order to only consider cells with both target and repair template reporters. BFP+mCherry+ cells were gated on GFP to determine trans-splicing frequency. Delivery of the CRISPR system via a multi-vector AAV approach showed significant increase in trans-splicing as measured by GFP+ fluorescence in flow cytometry (FIG. 13).

1.-221. (canceled)

222. A method of mediating a targeted trans-splicing event on a pre-mRNA in a cell, the method comprising introducing at least one portion of a targeting trans-splicing CRISPR/Cas system into the cell, wherein the CRISPR/Cas system comprises:
a) a Type VI Cas polypeptide;
b) a nucleic acid guide;
c) a trans-splicing template comprising a splice donor and/or a splice acceptor and an RNA sequence that hybridizes under physiological conditions to the specific RNA-binding domain.

223. The method of claim 222, wherein the CRISPR/Cas system further comprises a specific RNA-binding polypeptide that associates with the CRISPR/Cas system.

224. The method of claim 222, wherein the nucleic acid guide comprises RNA.

225. The method of claim 222, wherein the nucleic acid guide comprises DNA.

226. The method of claim 222, wherein the CRISPR/Cas system comprises one nucleic acid guide.

227. The method of claim 222, wherein the CRISPR/Cas system comprises more than one nucleic acid guide.

228. The method of claim 222, wherein the more than one nucleic acid guide target more than one nucleic acid of interest.

229. The method of claim 222, wherein the nucleic acid guide targets a splice acceptor (SA) site.

230. The method of claim 222, wherein the nucleic acid guide targets a splice donor (SD) site.

231. The method of claim 222, wherein the nucleic acid guide targets a region near the splice site.

232. The method of claim 222, wherein the nucleic acid guide targets a region within 200 nucleotides of the splice site.

233. The method of claim 222, wherein the nucleic acid guide targets a region less than or equal to 100 nucleotides of the splice site.

234. The method of claim 222, wherein the Type VI Cas polypeptide comprises a Cas13 polypeptide.

235. The method of claim 234, wherein the Cas13 polypeptide is a nuclease active Cas13a, Cas13b, or Cas13d polypeptide, or a nuclease-inactive Cas13b (dCas13b).

236. The method of 223, wherein the specific RNA-binding domain comprises a viral protein.

237. The method of claim 236, wherein the viral protein is a MS2 binding protein, a XN protein, a PP7 coat protein, or a QBeta coat protein.

238. The method of claim 222, wherein the trans-splicing event is regulated by a small molecule.

239. The method of claim 238, wherein the small molecule comprises abscisic acid (ABA), rapamycin or rapalog, FK506, Cyclosporine A, FK1012, Gibberellin3-AM, FKCsA, AP1903/AP20187, or Auxin.

240. The method of claim 222, wherein the CRISPR/Cas system comprises one or polynucleotides encoding the Type VI Cas polypeptide, the nucleic acid guide, and/or the trans-splicing template.

241. The method of claim 240, wherein the CRISPR/Cas system comprises one or more vectors comprising the one or more polynucleotides.


SEQUENCE LISTING (101 sequences)

SEQ ID NO: 1 - DNA, 3282 bp, synthetic construct
  misc_feature 1..3282: note = dPspCas13b nucleic acid sequence
  source 1..3282: mol_type = other DNA
atgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaacgcatacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatgcaaacaattaccccgacaaaggcgtggtggaaatcaaggccctgcctgagatcgccatgagcatcaagaaggcctttggggagtacgccatcatgaagggaagcctgcag

SEQ ID NO: 2 - protein, 1094 aa, synthetic construct
  REGION 1..1094: note = dPspCas13b amino acid sequence
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDANNYPDKGVVEIKALPEIAMSIKKAFGEYAIMKGSLQ

SEQ ID NO: 3 - DNA, 3717 bp, synthetic construct
  misc_feature 1..3717: note = dPspCas13b-SV40NLS-linker-MS2 nucleic acid sequence
  source 1..3717: mol_type = other DNA
atgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaacgcatacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatgcaaacaattaccccgacaaaggcgtggtggaaatcaaggccctgcctgagatcgccatgagcatcaagaaggcctttggggagtacgccatcatgaagggaagcctgcagccaaagaagaagcggaaggtcggtggatccggaggaggtggaagcatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctac

SEQ ID NO: 4 - protein, 1239 aa, synthetic construct
  REGION 1..1239: note = dPspCas13b-SV40NLS-linker-MS2 amino acid sequence
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDANNYPDKGVVEIKALPEIAMSIKKAFGEYAIMKGSLQPKKKRKVGGSGGGGSMASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 5 - DNA, 390 bp, synthetic construct
  misc_feature 1..390: note = MS2 coat protein nucleic acid sequence
  source 1..390: mol_type = other DNA
atggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctac

SEQ ID NO: 6 - protein, 130 aa, synthetic construct
  REGION 1..130: note = MS2 coat protein amino acid sequence
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 7 - protein, 22 aa, synthetic construct
  REGION 1..22: note = LambdaN amino acid sequence
MNARTRRRERRAEKQAQWKAAN

SEQ ID NO: 8 - protein, 133 aa, synthetic construct
  REGION 1..133: note = QBeta coat protein amino acid seqeunce
MAKLETVTLGNIGKDGKQTLVLNPRGVNPTNGVASLSQAGAVRALEKRVTVSVSQPSRNRKNYKVQVKIQNPTAGTANGSGDPSVTRQAYADVTFSFTQYSTDEERAFVRTELAALLASPLLIDAIDQLRPAY

SEQ ID NO: 9 - protein, 123 aa, synthetic construct
  REGION 1..123: note = PP7 coat protein amino acid sequence
MASKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGR

SEQ ID NO: 10 - DNA, 36 bp, synthetic construct
  misc_feature 1..36: note = PspCas13b direct repeat
  source 1..36: mol_type = other DNA
gttgtggaaggtccagttttgaggggctattacaac

SEQ ID NO: 11 - DNA, 56 bp, synthetic construct
  misc_feature 1..56: note = PspCas13 gRNA
  misc_difference 1..20: note = misc_feature - n is a, c, g, or t
  source 1..56: mol_type = other DNA
nnnnnnnnnnnnnnnnnnnngttgtggaaggtccagttttgaggggctattacaac

SEQ ID NO: 12 - DNA, 56 bp, synthetic construct
  misc_feature 1..56: note = PspCas13b gRNA
  source 1..56: mol_type = other DNA
tagcggtacaggtattcctggttgtggaaggtccagttttgaggggctattacaac

SEQ ID NO: 13 - DNA, 19 bp, synthetic construct
  misc_feature 1..19: note = MS2 hairpin
  source 1..19: mol_type = other DNA
acatgaggatcacccatgt

SEQ ID NO: 14 - DNA, 17 bp, synthetic construct
  misc_feature 1..17: note = boxB hairpin
  source 1..17: mol_type = other DNA
ggccctgaaaaagggcc

SEQ ID NO: 15 - DNA, 20 bp, synthetic construct
  misc_feature 1..20: note = QBeta hairpin
  source 1..20: mol_type = other DNA
atgcatgtctaagacagcat

SEQ ID NO: 16 - DNA, 25 bp, synthetic construct
  misc_feature 1..25: note = PP7 hairpin
  source 1..25: mol_type = other DNA
taaggagtttatatggaaaccctta

SEQ ID NO: 17 - DNA, 4170 bp, synthetic construct
  misc_feature 1..4170: note = LshCas13a nucleic acid sequence
  source 1..4170: mol_type = other DNA
atggggaacctgttcggacacaagcggtggtacgaggtgcgagataagaaagacttcaagatcaagcgaaaggtgaaagtcaagcggaattatgacgggaacaagtacattctgaacatcaacgaaaacaacaacaaagagaagatcgacaacaacaagttcatcagaaagtacatcaactacaagaaaaacgataatattctgaaggagttcactaggaaatttcatgcaggaaatatcctgttcaaactgaagggcaaagaagggatcattagaattgagaataacgacgacttcctggagacagaggaagtggtgctgtatatcgaggcctacggcaagagcgagaagctgaaagcactggggatcacaaagaaaaagatcattgacgaggccatcaggcagggcattactaaggacgataaaaagatcgagatcaagcgacaggagaacgaggaagagatcgaaattgacatccgggatgagtacactaataagaccctgaacgactgctccatcattctgcgcatcattgaaaacgatgaactggagacaaagaagagcatctacgagatcttcaagaacatcaatatgagcctgtataagatcatcgagaagatcatcgaaaacgagacagaaaaggtgtttgaaaatagatactacgaagagcacctgagggagaagctgctgaaagacgataagattgacgtgatcctgaccaacttcatggaaatccgggagaagatcaagtctaatctggagatcctgggcttcgtgaagttttacctgaacgtcggcggggacaaaaagaaaagtaaaaataagaaaatgctggtggaaaagattctgaacatcaatgtggatctgaccgtcgaggacattgccgatttcgtgatcaaggagctggaattttggaacatcacaaagcgcattgagaaagtgaagaaagtcaataacgagttcctggagaagcggagaaatcggacatacatcaagtcctatgtgctgctggacaagcacgaaaagtttaaaatcgagagagaaaacaagaaggataagatcgtgaagttctttgtcgagaacattaagaacaactctatcaaggaaaagattgagaagatcctggctgagttcaagatcgacgagctgattaagaaactggagaaggaactgaagaaagggaactgtgataccgagatcttcggaatctttaagaagcattacaaggtgaacttcgacagcaagaaattttccaagaaatctgatgaagagaaggagctgtataagatcatctacagatacctgaagggcagaattgaaaaaatcctggtgaacgagcagaaggtcagactgaagaaaatggagaagatcgagatcgaaaagattctgaatgaaagtatcctgtcagagaaaattctgaagagagtgaaacagtatacactggagcacattatgtacctggggaagctgaggcataacgacatcgatatgaccacagtgaatactgacgatttcagccgcctgcacgccaaggaagagctggacctggaactgatcaccttctttgccagcacaaatatggagctgaacaagatcttttcccgagaaaacatcaacaacgacgagaacatcgatttctttggaggcgaccgggagaagaactatgtgctggataagaaaatcctgaatagtaagatcaagatcatccgcgacctggatttcatcgataacaagaacaacatcacaaacaacttcattcgaaagtttacaaagatcggcactaatgaaaggaaccgcatcctgcatgccatttccaaagagagggacctgcaggggactcaggacgattacaacaaagtgatcaacatcattcagaatctgaagatctccgatgaagaggtgagcaaagctctgaacctggacgtggtctttaaggacaagaaaaacatcatcacaaagatcaatgacatcaagatctctgaagagaacaacaacgatatcaagtatctgcccagcttcagcaaagtgctgcccgaaatcctgaacctgtaccgcaacaatcccaagaatgagccttttgacacaatcgagactgaaaaaattgtgctgaacgctctgatctacgtcaataaggagctgtataagaaactgatcctggaggacgatctggaagagaacgagtccaagaatatcttcctgcaggaactgaagaaaaccctgggcaacattgacgaaatcgatgagaacatcatcgagaactactacaagaacgcacagatttctgccagtaaggggaacaacaaggcaatcaagaaatatcagaagaaagtgatcgagtgctacattggatatctgcgcaaaaactacgaagagctgttcgacttttcagacttcaagatgaacatccaggaaatcaagaaacagattaaggacatcaacgataacaagacttatgagtggatcaccgtgaaaaccagcgacaaaaccattgtcatcaacgacgatttcgagtacatcatttctatctttgcactgctgaacagtaatgccgtgattaataagatccgaaacagattcttcgccaccagcgtgtggctgaacacctcagaataccagaatatcattgacatcctggatgagattatgcagctgaataccctgcggaacgaatgcatcacagagaactggaatctgaacctggaagagttcattcagaagatgaaagagatcgaaaaggatttcgacgacttcaagatccagactaagaaagaaatcttcaacaactactacgaggacatcaagaacaacattctgaccgagtttaaagacgatatcaacggctgtgatgtgctggaaaagaaactggagaagattgtcatcttcgacgatgaaaccaagttcgagatcgacaagaaatccaacatcctgcaggatgaacagagaaagctgtctaacatcaacaagaaggacctgaagaaaaaggtggatcagtatatcaaggacaaagatcaggagatcaagtctaaaatcctgtgcaggatcattttcaacagtgactttctgaaaaagtacaagaaggaaatcgacaatctgattgaggatatggagtctgaaaatgagaacaagttccaggagatctactatcccaaggaacggaagaacgagctgtatatctacaaaaagaatctgttcctgaacatcggaaatcctaactttgacaagatctacggcctgattagcaacgacatcaagatggccgatgctaaattcctgtttaatatcgatggaaagaacatcagaaagaacaaaatcagtgagatcgacgctattctgaagaatctgaacgataaactgaacggctactcaaaggaatacaaggagaagtacatcaaaaagctgaaggagaacgacgatttctttgcaaagaacatccagaataagaactacaaatccttcgaaaaggactataaccgcgtgtctgagtacaaaaagattcgagatctggtcgagttcaactatctgaacaaaatcgagtcctacctgattgacatcaactggaagctggctattcagatggcaagattcgaaagggatatgcactatatcgtgaatggactgagggagctgggcatcattaagctgtcaggctataacaccgggatcagcagggcatacccaaagcgcaatggaagcgacggcttttacactaccacagcctactacaagttctttgatgaagagtcctacaagaagttcgagaagatttgctacgggtttggaatcgacctgagcgaaaattccgagatcaacaagcctgaaaatgagagcattcggaactatatctcccatttctacatcgtgagaaatccatttgccgactacagtattgctgagcagatcgatcgggtgagcaacctgctgtcatatagcacacgctacaacaattcaacttatgccagcgtgttcgaagtctttaaaaaggacgtgaatctggactacgatgagctgaaaaagaaattcaaactgatcggcaacaatgatattctggagcgcctgatgaagcccaagaaagtgagcgtcctggaactggagtcctacaacagtgactacattaagaatctgatcattgaactgctgaccaaaatcgagaatactaacgataccctgtga

SEQ ID NO: 18 - protein, 1389 aa, synthetic construct
  REGION 1..1389: note = LshCas13a amino acid sequence
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYEWITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 19 - DNA, 28 bp, synthetic construct
  misc_feature 1..28: note = LshCas13a direct repeat
  source 1..28: mol_type = other DNA
ccaccccaatatcgaaggggactaaaac

SEQ ID NO: 20 - DNA, 3456 bp, synthetic construct
  misc_feature 1..3456: note = LwaCas13a nucleic acid sequence
  source 1..3456: mol_type = other DNA
atgaaagtgaccaaggtcgacggcatcagccacaagaagtacatcgaagagggcaagctcgtgaagtccaccagcgaggaaaaccggaccagcgagagactgagcgagctgctgagcatccggctggacatctacatcaagaaccccgacaacgcctccgaggaagagaaccggatcagaagagagaacctgaagaagttctttagcaacaaggtgctgcacctgaaggacagcgtgctgtatctgaagaaccggaaagaaaagaacgccgtgcaggacaagaactatagcgaagaggacatcagcgagtacgacctgaaaaacaagaacagcttctccgtgctgaagaagatcctgctgaacgaggacgtgaactctgaggaactggaaatctttcggaaggacgtggaagccaagctgaacaagatcaacagcctgaagtacagcttcgaagagaacaaggccaactaccagaagatcaacgagaacaacgtggaaaaagtgggcggcaagagcaagcggaacatcatctacgactactacagagagagcgccaagcgcaacgactacatcaacaacgtgcaggaagccttcgacaagctgtataagaaagaggatatcgagaaactgtttttcctgatcgagaacagcaagaagcacgagaagtacaagatccgcgagtactatcacaagatcatcggccggaagaacgacaaagagaacttcgccaagattatctacgaagagatccagaacgtgaacaacatcaaagagctgattgagaagatccccgacatgtctgagctgaagaaaagccaggtgttctacaagtactacctggacaaagaggaactgaacgacaagaatattaagtacgccttctgccacttcgtggaaatcgagatgtcccagctgctgaaaaactacgtgtacaagcggctgagcaacatcagcaacgataagatcaagcggatcttcgagtaccagaatctgaaaaagctgatcgaaaacaaactgctgaacaagctggacacctacgtgcggaactgcggcaagtacaactactatctgcaagtgggcgagatcgccacctccgactttatcgcccggaaccggcagaacgaggccttcctgagaaacatcatcggcgtgtccagcgtggcctacttcagcctgaggaacatcctggaaaccgagaacgagaacggtatcaccggccggatgcggggcaagaccgtgaagaacaacaagggcgaagagaaatacgtgtccggcgaggtggacaagatctacaatgagaacaagcagaacgaagtgaaagaaaatctgaagatgttctacagctacgacttcaacatggacaacaagaacgagatcgaggacttcttcgccaacatcgacgaggccatcagcagcatcagacacggcatcgtgcacttcaacctggaactggaaggcaaggacatcttcgccttcaagaatatcgcccccagcgagatctccaagaagatgtttcagaacgaaatcaacgaaaagaagctgaagctgaaaatcttcaagcagctgaacagcgccaacgtgttcaactactacgagaaggatgtgatcatcaagtacctgaagaataccaagttcaacttcgtgaacaaaaacatccccttcgtgcccagcttcaccaagctgtacaacaagattgaggacctgcggaataccctgaagtttttttggagcgtgcccaaggacaaagaagagaaggacgcccagatctacctgctgaagaatatctactacggcgagttcctgaacaagttcgtgaaaaactccaaggtgttctttaagatcaccaatgaagtgatcaagattaacaagcagcggaaccagaaaaccggccactacaagtatcagaagttcgagaacatcgagaaaaccgtgcccgtggaatacctggccatcatccagagcagagagatgatcaacaaccaggacaaagaggaaaagaatacctacatcgactttattcagcagattttcctgaagggcttcatcgactacctgaacaagaacaatctgaagtatatcgagagcaacaacaacaatgacaacaacgacatcttctccaagatcaagatcaaaaaggataacaaagagaagtacgacaagatcctgaagaactatgagaagcacaatcggaacaaagaaatccctcacgagatcaatgagttcgtgcgcgagatcaagctggggaagattctgaagtacaccgagaatctgaacatgttttacctgatcctgaagctgctgaaccacaaagagctgaccaacctgaagggcagcctggaaaagtaccagtccgccaacaaagaagaaaccttcagcgacgagttggaactgatcaacctgctgaacctggacaacaacagagtgaccgaggacttcgagctggaagccaacgagatcggcaagttcctggacttcaacgaaaacaaaatcaaggaccggaaagagctgaaaaagttcgacaccaacaagatctatttcgacggcgagaacatcatcaagcaccgggccttctacaatatcaagaaatacggcatgctgaatctgctggaaaagatcgccgataaggccaagtataagatcagcctgaaagaactgaaagagtacagcaacaagaagaatgagattgaaaagaactacaccatgcagcagaacctgcaccggaagtacgccagacccaagaaggacgaaaagttcaacgacgaggactacaaagagtatgagaaggccatcggcaacatccagaagtacacccacctgaagaacaaggtggaattcaatgagctgaacctgctgcagggcctgctgctgaagatcctgcaccggctcgtgggctacaccagcatctgggagcgggacctgagattccggctgaagggcgagtttcccgagaaccactacatcgaggaaattttcaatttcgacaactccaagaatgtgaagtacaaaagcggccagatcgtggaaaagtatatcaacttctacaaagaactgtacaaggacaatgtggaaaagcggagcatctactccgacaagaaagtgaagaaactgaagcaggaaaaaaaggacctgtacatccggaactacattgcccacttcaactacatcccccacgccgagattagcctgctggaagtgctggaaaacctgcggaagctgctgtcctacgaccggaagctgaagaacgccatcatgaagtccatcgtggacattctgaaagaatacggcttcgtggccaccttcaagatcggcgctgacaagaagatcgaaatccagaccctggaatcagagaagatcgtgcacctgaagaatctgaagaaaaagaaactgatgaccgaccggaacagcgaggaactgtgcgaactcgtgaaagtcatgttcgagtacaaggccctggaa

SEQ ID NO: 21 - protein, 1152 aa, synthetic construct
  REGION 1..1152: note = LwaCas13 amino acid sequence
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENGITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 22 - DNA, 36 bp, synthetic construct
  misc_feature 1..36: note = LwaCas13a direct repeat
  source 1..36: mol_type = other DNA
gatttagactaccccaaaaacgaaggggactaaaac

SEQ ID NO: 23 - DNA, 3363 bp, synthetic construct
  misc_feature 1..3363: note = LseCas13a nucleic acid sequence
  source 1..3363: mol_type = other DNA
atgtggatcagcattaagaccctgatccaccatctgggcgtgctgttcttttgcgattacatgtataaccggagagagaagaaaatcatcgaagtcaaaacaatgaggatcactaaggtggaggtcgatcggaagaaagtgctgatcagccgggacaagaatggcgggaaactggtctatgagaacgaaatgcaggacaatacagagcagatcatgcaccataagaaaagctccttctacaaaagcgtggtcaacaagactatttgtcgccctgagcagaagcagatgaagaaactggtgcacggcctgctgcaggagaacagccaggaaaagatcaaggtgagcgatgtcactaagctgaatatctcaaacttcctgaatcacagattcaagaagagcctgtactatttcccagagaacagtcccgataagtcagaggaatacagaatcgaaattaatctgtctcagctgctggaggacagtctgaagaaacagcagggaaccttcatctgctgggagtccttttctaaggacatggaactgtatattaactgggccgagaactacatctctagtaagactaaactgattaagaaaagtatcaggaacaatcgcatccagtcaaccgagagtaggtcaggccagctgatggaccgctatatgaaggatattctgaacaagaacaagcccttcgatatccagagcgtgtccgagaaataccagctggaaaagctgacatccgccctgaaggctacttttaaagaagctaagaaaaacgacaaggagatcaattacaagctgaaatctaccctgcagaaccacgagaggcagatcattgaggaactgaaagagaatagcgaactgaaccagttcaatatcgagattcgcaagcatctggaaacttacttccctattaagaaaaccaacagaaaagtgggagatatcaggaatctggagatcggcgaaattcagaagatcgtgaaccaccgcctgaagaacaagattgtccagcgaatcctgcaggagggaaagctggcctcctatgagatcgaatctacagtgaactctaatagtctgcagaagatcaaaattgaggaagccttcgctctgaagtttatcaacgcttgcctgttcgcatctaacaatctgaggaatatggtgtaccccgtctgtaagaaagacattctgatgatcggcgagttcaagaacagcttcaaggagattaagcacaagaagttcatccgccagtggagccagttcttttcccaggaaattactgtggacgatatcgagctggcctcttggggactgcgaggagcaattgcccctatccggaacgagatcattcacctgaagaaacatagctggaagaaattctttaacaacccaactttcaaagtgaagaaatccaagatcattaatggcaagaccaaagacgtgaccagcgagttcctgtataaggaaaccctgttcaaggattacttttattctgagctggacagtgtgcccgaactgatcattaacaaaatggagtcaagcaagatcctggactactattcctctgatcagctgaaccaggtgttcaccattcctaattttgagctgtcactgctgacaagcgccgtgcccttcgctccttcctttaaaagagtctatctgaagggcttcgactaccagaaccaggatgaggcacagccagactataatctgaagctgaacatctacaatgagaaagccttcaacagcgaagcatttcaggcccagtactccctgtttaagatggtgtactatcaggtgttcctgccccagtttaccacaaacaatgatctgttcaagagttcagtggactttatcctgaccctgaacaaagagaggaagggctatgctaaggcattccaggatatccgcaaaatgaataaggacgagaaaccttccgaatacatgtcttatattcagagtcagctgatgctgtaccagaagaaacaggaggaaaaggagaaaatcaaccacttcgaaaagtttattaaccaggtgtttatcaaagggttcaacagctttattgagaagaataggctgacctacatctgccaccctaccaagaacacagtgccagagaacgataatatcgaaattccattccatacagacatggacgatagcaatatcgctttttggctgatgtgcaaactgctggatgcaaagcagctgtccgagctgcggaacgaaatgatcaagttcagctgtagcctgcagagtacagaggaaatttcaacttttaccaaggcaagagaagtgatcggcctggccctgctgaacggggagaaaggatgtaatgactggaaggagctgttcgacgataaagaagcctggaagaaaaacatgtcactgtatgtgagcgaggaactgctgcagtctctgccctacacccaggaggatgggcagacacctgtgattaatcggagtatcgacctggtcaagaaatatggaacagagactatcctggaaaagctgttcagctcctctgacgattataaggtgagcgcaaaagatattgccaagctgcacgagtacgacgtgacagaaaagattgctcagcaggaatccctgcataaacagtggatcgagaagccagggctggctcgggatagcgcatggactaagaaataccagaacgtgattaatgacatctccaactaccagtgggcaaagactaaagtggagctgacccaggtcagacacctgcatcagctgaccattgacctgctgtcccggctggccggatacatgtctatcgctgacagagatttccagtttagttcaaactacattctggagcgcgaaaatagcgaataccgagtgacaagctggattctgctgtccgagaacaagaacaagaacaagtacaacgattatgaactgtataacctgaagaatgcctccatcaaagtgagctccaagaatgatccccagctgaaagtcgacctgaagcagctgcggctgaccctggagtatctggaactgttcgacaatcgactgaaagagaagcggaacaatatcagtcactttaactacctgaatggccagctggggaactcaattctggaactgttcgacgatgccagagatgtgctgagctatgacaggaaactgaagaatgctgtctccaagtctctgaaagaaatcctgtctagtcatggcatggaggtgaccttcaagccactgtaccagactaaccaccatctgaagattgacaaactgcagcccaagaaaatccaccatctgggggagaagtctacagtgtcaagcaaccaggtcagtaacgaatactgtcagctggtgagaactctgctgaccatgaagtga

SEQ ID NO: 24 - protein, 1120 aa, synthetic construct
  REGION 1..1120: note = LseCas13a amino acid sequence
MWISIKTLIHHLGVLFFCDYMYNRREKKIIEVKTMRITKVEVDRKKVLISRDKNGGKLVYENEMQDNTEQIMHHKKSSFYKSVVNKTICRPEQKQMKKLVHGLLQENSQEKIKVSDVTKLNISNFLNHRFKKSLYYFPENSPDKSEEYRIEINLSQLLEDSLKKQQGTFICWESFSKDMELYINWAENYISSKTKLIKKSIRNNRIQSTESRSGQLMDRYMKDILNKNKPFDIQSVSEKYQLEKLTSALKATFKEAKKNDKEINYKLKSTLQNHERQIIEELKENSELNQFNIEIRKHLETYFPIKKTNRKVGDIRNLEIGEIQKIVNHRLKNKIVQRILQEGKLASYEIESTVNSNSLQKIKIEEAFALKFINACLFASNNLRNMVYPVCKKDILMIGEFKNSFKEIKHKKFIRQWSQFFSQEITVDDIELASWGLRGAIAPIRNEIIHLKKHSWKKFFNNPTFKVKKSKIINGKTKDVTSEFLYKETLFKDYFYSELDSVPELIINKMESSKILDYYSSDQLNQVFTIPNFELSLLTSAVPFAPSFKRVYLKGFDYQNQDEAQPDYNLKLNIYNEKAFNSEAFQAQYSLFKMVYYQVFLPQFTTNNDLFKSSVDFILTLNKERKGYAKAFQDIRKMNKDEKPSEYMSYIQSQLMLYQKKQEEKEKINHFEKFINQVFIKGFNSFIEKNRLTYICHPTKNTVPENDNIEIPFHTDMDDSNIAFWLMCKLLDAKQLSELRNEMIKFSCSLQSTEEISTFTKAREVIGLALLNGEKGCNDWKELFDDKEAWKKNMSLYVSEELLQSLPYTQEDGQTPVINRSIDLVKKYGTETILEKLFSSSDDYKVSAKDIAKLHEYDVTEKIAQQESLHKQWIEKPGLARDSAWTKKYQNVINDISNYQWAKTKVELTQVRHLHQLTIDLLSRLAGYMSIADRDFQFSSNYILERENSEYRVTSWILLSENKNKNKYNDYELYNLKNASIKVSSKNDPQLKVDLKQLRLTLEYLELFDNRLKEKRNNISHFNYLNGQLGNSILELFDDARDVLSYDRKLKNAVSKSLKEILSSHGMEVTFKPLYQTNHHLKIDKLQPKKIHHLGEKSTVSSNQVSNEYCQLVRTLLTMK

SEQ ID NO: 25 - DNA, 36 bp, synthetic construct
  misc_feature 1..36: note = LseCas13a direct repeat
  source 1..36: mol_type = other DNA
gtaagagactacctctatatgaaagaggactaaaac

SEQ ID NO: 26 - DNA, 4023 bp, synthetic construct
  misc_feature 1..4023: note = LbmCas13a nucleic acid sequence
  source 1..4023: mol_type = other DNA
atgcagatctccaaggtgaaccacaagcacgtggcagtgggacagaaggacagggagagaatcaccggcttcatctataatgacccagtgggcgatgagaagagcctggaggacgtggtggcaaagagggccaacgataccaaggtgctgttcaacgtgttcaacacaaaggacctgtacgattctcaggagagcgacaagtccgagaaggataaggagatcatctccaagggcgccaagttcgtggccaagtcttttaacagcgccatcacaatcctgaagaagcagaataagatctattccaccctgacatctcagcaggtcatcaaggagctgaaggacaagttcggcggcgcccgcatctacgacgatgacatcgaggaggccctgaccgagacactgaagaagtcttttagaaaggagaatgtgaggaacagcatcaaggtgctgatcgagaatgcagcaggcatccggagctccctgtccaaggacgaggaggagctgatccaggagtatttcgtgaagcagctggtggaggagtacaccaagacaaagctgcagaagaacgtggtgaagagcatcaagaatcagaacatggtcatccagcccgatagcgactcccaggtgctgtctctgagcgagtcccggagagagaagcagtctagcgccgtgtcctctgacaccctggtgaattgcaaggagaaggacgtgctgaaggccttcctgacagattacgccgtgctggatgaggacgagcgcaacagcctgctgtggaagctgcggaatctggtgaacctgtacttttatggctctgagagcatcagggattactcttataccaaggagaagagcgtgtggaaggagcacgacgagcagaaggccaacaagaccctgttcatcgatgagatctgccacatcacaaagatcggcaagaacggcaaggagcagaaggtgctggactatgaggagaataggagccgctgtcggaagcagaatatcaactactatcgctccgccctgaattacgccaagaacaataccagcggcatcttcgagaatgaggattccaaccacttttggattcacctgatcgagaacgaggtggagcggctgtataatggcatcgagaacggcgaggagttcaagtttgagacaggctacatctctgagaaagtgtggaaggccgtgatcaaccacctgagcatcaagtacatcgccctgggcaaggccgtgtataactatgccatgaaggagctgagctcccccggcgacatcgagcctggcaagatcgatgactcctatatcaacggcatcacctctttcgactacgagatcatcaaggccgaggagtccctgcagagagatatctctatgaatgtggtgtttgccacaaactacctggcctgcgccaccgtggatacagacaaggatttcctgctgtttagcaaggaggacatcaggtcctgcaccaagaaggatggcaacctgtgcaagaacatcatgcagttctggggcggctattccacatggaagaatttttgtgaggagtacctgaaggatgacaaggacgccctggagctgctgtactccctgaagtctatgctgtattctatgagaaactctagcttccacttttctaccgagaatgtggacaacggcagctgggatacagagctgatcggcaagctgttcgaggaggattgtaaccgcgccgcccggatcgagaaggagaagttctacaacaataacctgcacatgttttattcctctagcctgctggagaaggtgctggagagactgtactcctctcaccacgagagggccagccaggtgccttccttcaacagagtgtttgtgaggaagaatttcccaagctccctgtccgagcagagaatcacccccaagtttacagactccaaggatgagcagatctggcagtctgccgtgtactatctgtgcaaggagatctactataacgacttcctgcagagcaaggaggcctacaagctgtttagggagggcgtgaagaatctggacaagaacgatatcaataaccagaaggccgccgatagcttcaagcaggccgtggtgtactatggcaaggccatcggcaacgccaccctgtcccaggtgtgccaggccatcatgacagagtataataggcagaataacgacggcctgaagaagaagagcgcctacgccgagaagcagaacagcaacaagtacaagcactatcctctgttcctgaagcaggtgctgcagagcgccttttgggagtacctggatgagaacaaggagatctatggcttcatctctgcccagatccacaagagcaacgtggagatcaaggccgaggactttatcgccaattactctagccagcagtataagaagctggtggataaggtgaagaaaacccctgagctgcagaagtggtatacactgggccgcctgatcaatccacggcaggccaaccagttcctgggctccatcagaaattacgtgcagtttgtgaaggacatccagaggcgcgccaaggagaatggcaacccaatcaggaactactatgaggtgctggagtccgattctatcatcaagatcctggagatgtgcaccaagctgaatggcaccacaagcaacgacatccacgattacttccgcgacgaggatgagtacgccgagtatatctcccagttcgtgaactttggcgacgtgcactctggcgccgccctgaatgccttttgcaacagcgagtccgagggcaagaagaacggcatctactatgacggcatcaatcccatcgtgaatcggaactgggtgctgtgcaagctgtatggctcccctgatctgatctctaagatcatcagccgcgtgaatgagaacatgatccacgacttccacaagcaggaggatctgatccgggagtaccagatcaagggcatctgttctaacaagaaggagcagcaggacctgcgcacctttcaggtgctgaagaatcgcgtggagctgcgggatatcgtggagtacagcgagatcatcaacgagctgtatggccagctgatcaagtggtgctacctgagagagagggacctgatgtacttccagctgggctttcactacctgtgcctgaataacgcctcctctaaggaggccgattatatcaagatcaatgtggatgaccggaacatcagcggcgccatcctgtaccagatcgccgccatgtatatcaatggcctgcccgtgtactataagaaggatgacatgtacgtggccctgaagtccggcaagaaggcctctgacgagctgaatagcaacgagcagacctccaagaagatcaactacttcctgaagtatggcaataacatcctgggcgacaagaaggatcagctgtacctggccggcctggagctgttcgagaatgtggccgagcacgagaacatcatcatctttagaaatgagatcgaccacttccactacttttatgaccgcgatcggtccatgctggatctgtattctgaggtgtttgaccgcttctttacctacgatatgaagctgcggaagaatgtggtgaacatgctgtataacatcctgctggaccacaatatcgtgagctccttcgtgtttgagacaggcgagaagaaggtcggcaggggcgatagcgaagtgatcaagccttccgccaagatcagactgagggccaataacggcgtgtctagcgacgtgttcacctacaaagtgggcagcaaggatgagctgaagatcgccacactgccagccaagaacgaggagtttctgctgaatgtggccagactgatctactatcccgacatggaggccgtgtccgagaacatggtgagggagggcgtggtgaaggtggagaagtctaatgataagaagggcaagatcagcagaggctccaataccaggtcctctaaccagtctaagtacaacaacaagagcaagaacagaatgaactactctatgggcagcatcttcgagaagatggacctgaagtttgattga

SEQ ID NO: 27 - protein, 1340 aa, synthetic construct
  REGION 1..1340: note = LbmCas13a amino acid sequence
MQISKVNHKHVAVGQKDRERITGFIYNDPVGDEKSLEDVVAKRANDTKVLFNVFNTKDLYDSQESDKSEKDKEIISKGAKFVAKSFNSAITILKKQNKIYSTLTSQQVIKELKDKFGGARIYDDDIEEALTETLKKSFRKENVRNSIKVLIENAAGIRSSLSKDEEELIQEYFVKQLVEEYTKTKLQKNVVKSIKNQNMVIQPDSDSQVLSLSESRREKQSSAVSSDTLVNCKEKDVLKAFLTDYAVLDEDERNSLLWKLRNLVNLYFYGSESIRDYSYTKEKSVWKEHDEQKANKTLFIDEICHITKIGKNGKEQKVLDYEENRSRCRKQNINYYRSALNYAKNNTSGIFENEDSNHFWIHLIENEVERLYNGIENGEEFKFETGYISEKVWKAVINHLSIKYIALGKAVYNYAMKELSSPGDIEPGKIDDSYINGITSFDYEIIKAEESLQRDISMNVVFATNYLACATVDTDKDFLLFSKEDIRSCTKKDGNLCKNIMQFWGGYSTWKNFCEEYLKDDKDALELLYSLKSMLYSMRNSSFHFSTENVDNGSWDTELIGKLFEEDCNRAARIEKEKFYNNNLHMFYSSSLLEKVLERLYSSHHERASQVPSFNRVFVRKNFPSSLSEQRITPKFTDSKDEQIWQSAVYYLCKEIYYNDFLQSKEAYKLFREGVKNLDKNDINNQKAADSFKQAVVYYGKAIGNATLSQVCQAIMTEYNRQNNDGLKKKSAYAEKQNSNKYKHYPLFLKQVLQSAFWEYLDENKEIYGFISAQIHKSNVEIKAEDFIANYSSQQYKKLVDKVKKTPELQKWYTLGRLINPRQANQFLGSIRNYVQFVKDIQRRAKENGNPIRNYYEVLESDSIIKILEMCTKLNGTTSNDIHDYFRDEDEYAEYISQFVNFGDVHSGAALNAFCNSESEGKKNGIYYDGINPIVNRNWVLCKLYGSPDLISKIISRVNENMIHDFHKQEDLIREYQIKGICSNKKEQQDLRTFQVLKNRVELRDIVEYSEIINELYGQLIKWCYLRERDLMYFQLGFHYLCLNNASSKEADYIKINVDDRNISGAILYQIAAMYINGLPVYYKKDDMYVALKSGKKASDELNSNEQTSKKINYFLKYGNNILGDKKDQLYLAGLELFENVAEHENIIIFRNEIDHFHYFYDRDRSMLDLYSEVFDRFFTYDMKLRKNVVNMLYNILLDHNIVSSFVFETGEKKVGRGDSEVIKPSAKIRLRANNGVSSDVFTYKVGSKDELKIATLPAKNEEFLLNVARLIYYPDMEAVSENMVREGVVKVEKSNDKKGKISRGSNTRSSNQSKYNNKSKNRMNYSMGSIFEKMDLKFD

SEQ ID NO: 28 - DNA, 35 bp, synthetic construct
  misc_feature 1..35: note = LbmCas13a direct repeat
  source 1..35: mol_type = other DNA
gtattgagaaaagccagatatagttggcaatagac

SEQ ID NO: 29 - DNA, 4314 bp, synthetic construct
  misc_feature 1..4314: note = LbnCas13a nucleic acid sequence
  source 1..4314: mol_type = other DNA
atgaagatcagcaaggtgcgggaggagaacagaggcgccaagctgaccgtgaatgccaagacagccgtggtgtctgagaaccggagccaggagggcatcctgtataatgacccaagcaggtacggcaagtcccgcaagaacgacgaggatagggaccgctatatcgagagcagactgaagagctccggcaagctgtaccggatctttaacgaggataagaataagcgcgagacagacgagctgcagtggttcctgtccgagatcgtgaagaagatcaaccggagaaatggcctggtgctgtccgacatgctgtctgtggacgatagagccttcgagaaggcctttgagaagtacgccgagctgtcttataccaacaggcgcaataaggtgtccggctctcccgccttcgagacatgcggagtggatgcagcaacagcagagcggctgaagggaatcatctccgagacaaacttcatcaacagaatcaagaacaacatcgacaacaaggtgtctgaggacatcatcgatcggatcatcgccaagtacctgaagaagagcctgtgccgggagagagtgaagagaggcctgaagaagctgctgatgaatgcctttgatctgccatactctgatcccgacatcgatgtgcagagggacttcatcgattatgtgctggaggacttttaccacgtgcgggccaagagccaggtgagcagatccatcaagaacatgaatatgcccgtgcagcctgagggcgacggcaagttcgccatcaccgtgagcaagggcggcacagagtctggcaacaagcgcagcgccgagaaggaggccttcaagaagtttctgagcgattacgcctccctggacgagagggtgcgcgacgatatgctgcggagaatgaggcgcctggtggtgctgtacttttatggctccgacgattctaagctgagcgatgtgaacgagaagttcgacgtgtgggaggaccacgcagcccggagagtggacaatagggagttcatcaagctgccactggagaacaagctggccaatggcaagaccgacaaggatgccgagcggatcagaaagaacacagtgaaggagctgtatagaaaccagaatatcggctgctacaggcaggccgtgaaggccgtggaggaggacaacaatggccggtactttgacgataagatgctgaacatgttctttatccacagaatcgagtatggcgtggagaagatctacgccaatctgaagcaggtgaccgagttcaaggcccgcacaggctacctgagcgagaagatctggaaggatctgatcaactacatctctatcaagtatatcgccatgggcaaggccgtgtataactatgccatggatgagctgaatgcctccgacaagaaggagatcgagctgggcaagatctccgaggagtatctgtctggcatctctagcttcgactacgagctgatcaaggccgaggagatgctgcagagggagacagccgtgtacgtggcctttgccgcaaggcacctgtcctctcagacagtggagctggattccgagaactctgacttcctgctgctgaagcctaagggcaccatggacaagaacgataagaataagctggcctccaacaatatcctgaattttctgaaggataaggagacactgcgggacacaatcctgcagtatttcggcggccactctctgtggacagatttcccatttgacaagtacctggccggcggcaaggacgatgtggattttctgaccgacctgaaggatgtgatctatagcatgcggaacgactccttccactacgccacagagaaccacaacaatggcaagtggaataaggagctgatctccgccatgtttgagcacgagacagagagaatgacagtggtcatgaaggacaagttctattctaacaatctgcccatgttttacaagaacgacgatctgaagaagctgctgatcgacctgtataaggacaatgtggagagagcctctcaggtgcccagcttcaacaaggtgtttgtgcgcaagaatttccctgccctggtgcgggacaaggataacctgggcatcgagctggatctgaaggccgacgccgataagggcgagaatgagctgaagttctacaacgccctgtactacatgttcaaggagatctactacaacgccttcctgaacgacaagaatgtgagggagcggttcatcaccaaggccacaaaggtggccgacaactatgataggaataaggagcgcaacctgaaggataggatcaagagcgccggctccgacgagaagaagaagctgcgcgagcagctgcagaattacatcgccgagaacgatttcggccagaggatcaagaatatcgtgcaggtgaaccctgactataccctggcccagatctgccagctgatcatgacagagtacaaccagcagaacaatggctgtatgcagaagaagagcgccgcccggaaggatatcaataaggactcctaccagcactataagatgctgctgctggtgaacctgagaaaggccttcctggagtttatcaaggagaattatgcctttgtgctgaagccctacaagcacgacctgtgcgataaggccgacttcgtgcctgattttgccaagtacgtgaagccctacgccggcctgatcagcagggtggcaggcagctccgagctgcagaagtggtatatcgtgtcccgctttctgtctcctgcccaggccaaccacatgctgggcttcctgcactcctacaagcagtacgtgtgggacatctacaggcgcgcctctgagacaggcaccgagatcaatcacagcatcgccgaggataagatcgccggcgtggacatcaccgacgtggatgccgtgatcgatctgagcgtgaagctgtgcggcacaatctctagcgagatctccgactacttcaaggacgatgaggtgtacgccgagtatatctcctcttacctggattttgagtatgacggcggcaactacaaggatagcctgaataggttctgtaactccgatgccgtgaatgaccagaaggtggccctgtactatgacggcgagcaccctaagctgaaccgcaatatcatcctgagcaagctgtacggcgagcggagatttctggagaagatcaccgatcgggtgagccggagcgacatcgtggagtactataagctgaagaaggagacaagccagtaccagacaaagggcatcttcgatagcgaggacgagcagaagaacatcaagaagtttcaggagatgaagaatatcgtggagttcagagatctgatggactatagcgagatcgccgatgagctgcagggccagctgatcaactggatctacctgcgggagcgggacctgatgaatttccagctgggctaccactatgcctgcctgaacaatgattccaacaagcaggccacctatgtgacactggactaccagggcaagaagaatcggaagatcaacggcgccatcctgtatcagatctgtgccatgtatatcaatggcctgcctctgtactatgtggacaaggatagctccgagtggaccgtgtctgacggcaaggagagcacaggcgccaagatcggcgagttctacagatatgccaagtcctttgagaacacctctgattgctacgccagcggcctggagatctttgagaatatcagcgagcacgacaacatcacagagctgaggaattatatcgagcactttcgctactacagcagcttcgaccggagcttcctgggcatctactctgaggtgttcgatcggttctttacctacgacctgaagtatagaaagaacgtgccaacaatcctgtataacatcctgctgcagcacttcgtgaacgtgaggttcgagttcgtgagcggcaagaagatgatcggcatcgataagaaggaccgcaagatcgccaaggagaaggagtgtgcccggatcaccatcagagagaagaacggcgtgtatagcgagcagtttacctacaagctgaagaatggcacagtgtatgtggatgccagggacaagagatacctgcagtccatcatcaggctgctgttctatccagagaaggtgaacatggatgagatgatcgaggtgaaggagaagaagaagccctccgacaacaataccggcaagggctactctaagagggatcgccagcaggaccgcaaggagtacgataagtataaggagaagaagaagaaggagggcaatttcctgagcggcatgggcggcaacatcaattgggacgagatcaatgcccagctgaagaactga

SEQ ID NO: 30 - protein, 1437 aa, synthetic construct
  REGION 1..1437: note = LbnCas13a amino acid sequence
MKISKVREENRGAKLTVNAKTAVVSENRSQEGILYNDPSRYGKSRKNDEDRDRYIESRLKSSGKLYRIFNEDKNKRETDELQWFLSEIVKKINRRNGLVLSDMLSVDDRAFEKAFEKYAELSYTNRRNKVSGSPAFETCGVDAATAERLKGIISETNFINRIKNNIDNKVSEDIIDRIIAKYLKKSLCRERVKRGLKKLLMNAFDLPYSDPDIDVQRDFIDYVLEDFYHVRAKSQVSRSIKNMNMPVQPEGDGKFAITVSKGGTESGNKRSAEKEAFKKFLSDYASLDERVRDDMLRRMRRLVVLYFYGSDDSKLSDVNEKFDVWEDHAARRVDNREFIKLPLENKLANGKTDKDAERIRKNTVKELYRNQNIGCYRQAVKAVEEDNNGRYFDDKMLNMFFIHRIEYGVEKIYANLKQVTEFKARTGYLSEKIWKDLINYISIKYIAMGKAVYNYAMDELNASDKKEIELGKISEEYLSGISSFDYELIKAEEMLQRETAVYVAFAARHLSSQTVELDSENSDFLLLKPKGTMDKNDKNKLASNNILNFLKDKETLRDTILQYFGGHSLWTDFPFDKYLAGGKDDVDFLTDLKDVIYSMRNDSFHYATENHNNGKWNKELISAMFEHETERMTVVMKDKFYSNNLPMFYKNDDLKKLLIDLYKDNVERASQVPSFNKVFVRKNFPALVRDKDNLGIELDLKADADKGENELKFYNALYYMFKEIYYNAFLNDKNVRERFITKATKVADNYDRNKERNLKDRIKSAGSDEKKKLREQLQNYIAENDFGQRIKNIVQVNPDYTLAQICQLIMTEYNQQNNGCMQKKSAARKDINKDSYQHYKMLLLVNLRKAFLEFIKENYAFVLKPYKHDLCDKADFVPDFAKYVKPYAGLISRVAGSSELQKWYIVSRFLSPAQANHMLGFLHSYKQYVWDIYRRASETGTEINHSIAEDKIAGVDITDVDAVIDLSVKLCGTISSEISDYFKDDEVYAEYISSYLDFEYDGGNYKDSLNRFCNSDAVNDQKVALYYDGEHPKLNRNIILSKLYGERRFLEKITDRVSRSDIVEYYKLKKETSQYQTKGIFDSEDEQKNIKKFQEMKNIVEFRDLMDYSEIADELQGQLINWIYLRERDLMNFQLGYHYACLNNDSNKQATYVTLDYQGKKNRKINGAILYQICAMYINGLPLYYVDKDSSEWTVSDGKESTGAKIGEFYRYAKSFENTSDCYASGLEIFENISEHDNITELRNYIEHFRYYSSFDRSFLGIYSEVFDRFFTYDLKYRKNVPTILYNILLQHFVNVRFEFVSGKKMIGIDKKDRKIAKEKECARITIREKNGVYSEQFTYKLKNGTVYVDARDKRYLQSIIRLLFYPEKVNMDEMIEVKEKKKPSDNNTGKGYSKRDRQQDRKEYDKYKEKKKKEGNFLSGMGGNINWDEINAQLKN

SEQ ID NO: 31 - DNA, 35 bp, synthetic construct
  misc_feature 1..35: note = LbnCas13a direct repeat
  source 1..35: mol_type = other DNA
gttgatgagaagagcccaagatagagggcaataac

SEQ ID NO: 32 - DNA, 4158 bp, synthetic construct
  misc_feature 1..4158: note = CamCas13a nucleic acid sequence
  source 1..4158: mol_type = other DNA
atgaagttctctaaggtggaccacacccggagcgccgtgggcatccagaaggccaccgactctgtgcacggcatgctgtataccgaccccaagaagcaggaagtgaatgatctggacaagaggtttgatcagctgaacgtgaaggccaagcgcctgtataacgtgttcaatcagtccaaggccgaggaggacgatgacgagaagcggtttggcaaggtggtgaagaagctgaacagagagctgaaggacctgctgttccaccgggaggtgagcagatacaactccatcggcaatgccaagtataactactatggcatcaagtccaaccctgaggagatcgtgtctaatctgggcatggtggagagcctgaagggagagcgggacccccagaaagtgatcagcaagctgctgctgtactatctgagaaagggcctgaagcctggcaccgacggcctgaggatgatcctggaggcctcctgcggcctgcgcaagctgtctggcgatgagaaggagctgaaggtgttcctgcagaccctggatgaggactttgagaagaaaaccttcaagaagaacctgatccggtctatcgagaaccagaatatggccgtgcagccaagcaacgagggcgaccccatcatcggcatcacccagggccggtttaatagccagaagaacgaggagaagtccgccatcgagagaatgatgtctatgtacgccgatctgaacgaggaccacagggaggatgtgctgcgcaagctgcggagactgaatgtgctgtatttcaacgtggacaccgagaaaaccgaggagcccaccctgcctggagaggtggatacaaatccagtgtttgaagtgtggcacgaccacgagaagggcaaggagaacgatagacagtttgccaccttcgccaagatcctgaccgaggacagggagacacgcaagaaggagaagctggccgtgaaggaggccctgaatgacctgaagagcgccatcagggatcacaacatcatggcctaccgctgttccatcaaggtgaccgagcaggataaggacggcctgttctttgaggaccagcggatcaatagattctggattcaccacatcgagtctgccgtggagagaatcctggccagcatcaaccccgagaagctgtataagctgcgcatcggctatctgggcgagaaagtgtggaaggatctgctgaattacctgagcatcaagtatatcgccgtgggcaaggccgtgttccactttgccatggaggacctgggcaagaccggccaggatatcgagctgggcaagctgagcaactccgtgtctggcggcctgaccagcttcgactacgagcagatccgggccgatgagacactgcagagacagctgtccgtggaggtggcctttgccgccaacaatctgttcagggcagtggtgggacagaccggcaagaagatcgagcagagcaagtccgaggagaatgaggaggactttctgctgtggaaggccgagaagatcgccgagtccatcaagaaggagggcgagggcaacacactgaagtctatcctgcagttctttggcggcgccagctcctgggatctgaatcacttctgcgcagcctacggcaacgagtctagcgccctgggctatgagacaaagtttgccgatgacctgaggaaggccatctactccctgcgcaatgagacattccactttaccacactgaacaagggctcttttgactggaatgccaagctgatcggcgatatgttcagccacgaggcagcaaccggcatcgcagtggagaggacacgcttttacagcaacaatctgcctatgttctatcgggagtccgacctgaagagaatcatggatcacctgtataacacctatcaccctcgcgccagccaggtgccatctttcaacagcgtgtttgtgaggaagaattttcgcctgttcctgtccaacaccctgaacaccaatacatctttcgacacagaggtgtaccagaagtgggagtccggcgtgtactatctgtttaaggagatctactacaactctttcctgccaagcggcgacgcccaccacctgttctttgagggcctgaggcgcatcaggaaggaggccgataatctgcccatcgtgggcaaggaggccaagaagcgcaacgccgtgcaggactttggccggagatgcgatgagctgaagaacctgtctctgagcgccatctgtcagatgatcatgaccgagtacaatgagcagaacaatggcaacaggaaggtgaagagcacacgggaggacaagagaaagcccgatatcttccagcactacaagatgctgctgctgcgcaccctgcaggaggcctttgccatctatatcaggcgcgaggagttcaagtttatcttcgacctgcccaagacactgtacgtgatgaagcccgtggaggagtttctgcctaactggaagtccggcatgttcgactctctggtggagcgggtgaagcagtcccctgatctgcagagatggtatgtgctgtgcaagttcctgaatggccggctgctgaaccagctgagcggcgtgatcagatcctacatccagtttgcaggcgacatccagcggagagcaaaggcaaaccacaatcggctgtatatggataatacccagagagtggagtactattctaacgtgctggaggtggtggacttctgcatcaagggcacatccaggttctctaacgtgttcagcgattacttccgcgatgaggacgcctacgccgattatctggacaactatctgcagtttaaggacgagaagatcgccgaggtgagcagcttcgccgccctgaaaaccttctgtaacgaggaggaggtgaaggccggcatctacatggacggcgagaatcctgtgatgcagaggaacatcgtgatggccaagctgttcggccctgatgaggtgctgaagaatgtggtgccaaaggtgacacgggaggagatcgaggagtactatcagctggagaagcagatcgccccatacagacagaacggctattgtaagtccgaggaggaccagaagaagctgctgaggttccagcgcatcaagaatcgcgtggagtttcagaccatcacagagttctctgagatcatcaacgagctgctgggccagctgatctcctggtcttttctgcgggagagagatctgctgtactttcagctgggcttccactatctgtgcctgcacaatgacaccgagaagcctgccgagtacaaggagatcagccgggaggatggcacagtgatcagaaacgccatcctgcaccaggtggcagcaatgtacgtgggaggcctgccagtgtataccctggccgacaagaagctggccgccttcgagaagggagaggcagactgtaagctgagcatctccaaggatacagccggcgccggcaagaagatcaaggatttctttcggtactccaagtatgtgctgatcaaggacagaatgctgaccgatcagaaccagaagtacacaatctatctggccggcctggagctgttcgagaataccgatgagcacgacaacatcacagacgtgcggaagtacgtggatcactttaagtactatgccacctctgacgagaatgccatgagcatcctggatctgtattccgagatccacgacagattctttacatacgatatgaagtaccagaagaacgtggccaatatgctggagaacatcctgctgaggcacttcgtgctgatccgccccgagttctttaccggcagcaagaaggtcggcgagggcaagaagatcacatgcaaggccagggcccagatcgagatcgccgagaatggcatgcgctccgaggactttacctacaagctgagcgatggcaagaagaatatctccacatgtatgatcgccgccagggaccagaagtatctgaacaccgtggcccgcctgctgtactatccacacgaggccaagaagagcatcgtggacacacgggagaagaagaacaacaagaaaaccaatagaggcgatggcacattcaacaagcagaagggcaccgccaggaaggagaaggacaatggcccccgcgagtttaacgataccggcttctccaatacaccatttgccggcttcgatccctttagaaactcttga

SEQ ID NO: 33 - protein, 1385 aa, synthetic construct
  REGION 1..1385: note = CamCas13a amino acid sequence
MKFSKVDHTRSAVGIQKATDSVHGMLYTDPKKQEVNDLDKRFDQLNVKAKRLYNVFNQSKAEEDDDEKRFGKVVKKLNRELKDLLFHREVSRYNSIGNAKYNYYGIKSNPEEIVSNLGMVESLKGERDPQKVISKLLLYYLRKGLKPGTDGLRMILEASCGLRKLSGDEKELKVFLQTLDEDFEKKTFKKNLIRSIENQNMAVQPSNEGDPIIGITQGRFNSQKNEEKSAIERMMSMYADLNEDHREDVLRKLRRLNVLYFNVDTEKTEEPTLPGEVDTNPVFEVWHDHEKGKENDRQFATFAKILTEDRETRKKEKLAVKEALNDLKSAIRDHNIMAYRCSIKVTEQDKDGLFFEDQRINRFWIHHIESAVERILASINPEKLYKLRIGYLGEKVWKDLLNYLSIKYIAVGKAVFHFAMEDLGKTGQDIELGKLSNSVSGGLTSFDYEQIRADETLQRQLSVEVAFAANNLFRAVVGQTGKKIEQSKSEENEEDFLLWKAEKIAESIKKEGEGNTLKSILQFFGGASSWDLNHFCAAYGNESSALGYETKFADDLRKAIYSLRNETFHFTTLNKGSFDWNAKLIGDMFSHEAATGIAVERTRFYSNNLPMFYRESDLKRIMDHLYNTYHPRASQVPSFNSVFVRKNFRLFLSNTLNTNTSFDTEVYQKWESGVYYLFKEIYYNSFLPSGDAHHLFFEGLRRIRKEADNLPIVGKEAKKRNAVQDFGRRCDELKNLSLSAICQMIMTEYNEQNNGNRKVKSTREDKRKPDIFQHYKMLLLRTLQEAFAIYIRREEFKFIFDLPKTLYVMKPVEEFLPNWKSGMFDSLVERVKQSPDLQRWYVLCKFLNGRLLNQLSGVIRSYIQFAGDIQRRAKANHNRLYMDNTQRVEYYSNVLEVVDFCIKGTSRFSNVFSDYFRDEDAYADYLDNYLQFKDEKIAEVSSFAALKTFCNEEEVKAGIYMDGENPVMQRNIVMAKLFGPDEVLKNVVPKVTREEIEEYYQLEKQIAPYRQNGYCKSEEDQKKLLRFQRIKNRVEFQTITEFSEIINELLGQLISWSFLRERDLLYFQLGFHYLCLHNDTEKPAEYKEISREDGTVIRNAILHQVAAMYVGGLPVYTLADKKLAAFEKGEADCKLSISKDTAGAGKKIKDFFRYSKYVLIKDRMLTDQNQKYTIYLAGLELFENTDEHDNITDVRKYVDHFKYYATSDENAMSILDLYSEIHDRFFTYDMKYQKNVANMLENILLRHFVLIRPEFFTGSKKVGEGKKITCKARAQIEIAENGMRSEDFTYKLSDGKKNISTCMIAARDQKYLNTVARLLYYPHEAKKSIVDTREKKNNKKTNRGDGTFNKQKGTARKEKDNGPREFNDTGFSNTPFAGFDPFRNS

SEQ ID NO: 34 - DNA, 35 bp, synthetic construct
  misc_feature 1..35: note = CamCas13a direct repeat
  source 1..35: mol_type = other DNA
gtctattgccctctatatcgggctgttctccaaac

SEQ ID NO: 35 - DNA, 3354 bp, synthetic construct
  misc_feature 1..3354: note = CgaCas13a nucleic acid sequence
  source 1..3354: mol_type = other DNA
atgcggatcacaaaggtgaagatcaagctggacaacaagctgtaccaggtgaccatgcagaaggaggagaagtatggcacactgaagctgaatgaggagagcaggaagtccaccgccgagatcctgcgcctgaagaaggccagcttcaacaagtcttttcacagcaagaccatcaattcccagaaggagaacaagaatgccacaatcaagaagaacggcgactacatcagccagatcttcgagaagctggtgggcgtggataccaacaagaatatcaggaagcccaagatgtccctgacagacctgaaggatctgcctaagaaggacctggccctgttcatcaagcgcaagtttaagaacgacgatatcgtggagatcaagaatctggatctgatctccctgttttataatgccctgcagaaggtgccaggcgagcacttcaccgacgagtcttgggccgatttttgccaggagatgatgccctaccgggagtataagaacaagttcatcgagagaaagatcatcctgctggccaactccatcgagcagaataagggcttctctatcaatcccgagacattcagcaagcggaagagagtgctgcaccagtgggcaatcgaggtgcaggagaggggcgacttttccatcctggatgagaagctgtctaagctggccgagatctacaacttcaagaagatgtgcaagagagtgcaggacgagctgaacgatctggagaagagcatgaagaagggcaagaatcctgagaaggagaaggaggcctataagaagcagaagaactttaagatcaagaccatctggaaggactacccatataagacacacatcggcctgatcgagaagatcaaggagaatgaggagctgaaccagttcaatatcgagatcggcaagtacttcgagcactattttccaatcaagaaggagagatgcaccgaggatgagccctactatctgaacagcgagacaatcgccaccacagtgaactaccagctgaagaatgccctgatctcctacctgatgcagatcggcaagtataagcagtttggcctggagaatcaggtgctggactccaagaagctgcaggagatcggcatctatgagggcttccagaccaagtttatggatgcctgcgtgttcgccacaagctccctgaagaacatcatcgagcctatgcggagcggcgacatcctgggcaagagagagtttaaggaggccatcgccacatctagcttcgtgaattaccaccacttctttccctatttcccttttgagctgaagggcatgaaggatagggagtccgagctgatcccatttggcgagcagaccgaggccaagcagatgcagaacatctgggccctgaggggctctgtgcagcagatccgcaatgagatcttccacagctttgacaagaaccagaagttcaatctgccccagctggacaagagcaacttcgagtttgatgcctccgagaacagcaccggcaagtctcagagctacatcgagacagattataagttcctgtttgaggccgagaagaaccagctggagcagttctttatcgagcgcatcaagtcctctggcgccctggagtactatcccctgaagtccctggagaagctgttcgccaagaaggagatgaagtttagcctgggctcccaggtggtggccttcgcccctagctacaagaagctggtgaagaagggccactcctatcagaccgccacagagggcaccgccaactacctgggcctgtcctactataataggtatgagctgaaggaggagtcttttcaggcccagtactatctgctgaagctgatctaccagtacgtgttcctgcctaacttttcccagggcaattctccagccttccgcgagacagtgaaggccatcctgcggatcaacaaggacgaggccagaaagaagatgaagaagaataagaagttcctgaggaagtacgcctttgagcaggtgcgcgagatggagttcaaggagacacccgaccagtacatgagctatctgcagtccgagatgagggaggagaaggtgcgcaaggccgagaagaacgataagggcttcgagaagaacatcaccatgaattttgagaagctgctgatgcagatcttcgtgaagggcttcgacgtgttcctgaccacatttgccggcaaggagctgctgctgagctccgaggagaaagtgatcaaggagacagagatctccctgtctaagaagatcaacgagcgggagaaaaccctgaaggccagcatccaggtggagcaccagctggtggccaccaattctgccatcagctactggctgttctgcaagctgctggactcccggcacctgaacgagctgagaaatgagatgatcaagttcaagcagagccggatcaagttcaaccacacacagcacgccgagctgatccagaatctgctgcctatcgtggagctgaccatcctgtctaacgactacgatgagaagaacgactcccagaatgtggacgtgagcgcctattttgaggataagagcctgtacgagacagccccttatgtgcagaccgacgataggacacgcgtgtctttcaggccaatcctgaagctggagaagtaccacacaaagagcctgatcgaggccctgctgaaggacaacccacagtttcgcgtggccgccaccgatatccaggagtggatgcacaagagggaggagatcggcgagctggtggagaagcgcaagaatctgcacaccgagtgggcagagggacagcagacactgggagcagagaagcgggaggagtacagagactattgtaagaagatcgatcggttcaactggaaggccaataaggtgaccctgacatacctgtctcagctgcactatctgatcacagacctgctgggcagaatggtgggcttcagcgccctgtttgagagggatctggtgtacttcagccgctccttttctgagctgggcggcgagacataccacatcagcgattataagaacctgtccggcgtgctgcggctgaatgccgaggtgaagcccatcaagatcaagaacatcaaagtgatcgacaacgaggagaatccttacaagggcaatgagccagaggtgaagcccttcctggatcggctgcacgcctatctggagaacgtgatcggcatcaaggccgtgcacggcaagatcagaaatcagacagcccacctgtccgtgctgcagctggagctgtctatgatcgagagcatgaacaatctgcgggacctgatggcctacgatagaaagctgaagaacgccgtgaccaagtctatgatcaagatcctggacaagcacggctga

SEQ ID NO: 36 - protein, 1117 aa, synthetic construct
  REGION 1..1117: note = CgaCas13a amino acid sequence
MRITKVKIKLDNKLYQVTMQKEEKYGTLKLNEESRKSTAEILRLKKASFNKSFHSKTINSQKENKNATIKKNGDYISQIFEKLVGVDTNKNIRKPKMSLTDLKDLPKKDLALFIKRKFKNDDIVEIKNLDLISLFYNALQKVPGEHFTDESWADFCQEMMPYREYKNKFIERKIILLANSIEQNKGFSINPETFSKRKRVLHQWAIEVQERGDFSILDEKLSKLAEIYNFKKMCKRVQDELNDLEKSMKKGKNPEKEKEAYKKQKNFKIKTIWKDYPYKTHIGLIEKIKENEELNQFNIEIGKYFEHYFPIKKERCTEDEPYYLNSETIATTVNYQLKNALISYLMQIGKYKQFGLENQVLDSKKLQEIGIYEGFQTKFMDACVFATSSLKNIIEPMRSGDILGKREFKEAIATSSFVNYHHFFPYFPFELKGMKDRESELIPFGEQTEAKQMQNIWALRGSVQQIRNEIFHSFDKNQKFNLPQLDKSNFEFDASENSTGKSQSYIETDYKFLFEAEKNQLEQFFIERIKSSGALEYYPLKSLEKLFAKKEMKFSLGSQVVAFAPSYKKLVKKGHSYQTATEGTANYLGLSYYNRYELKEESFQAQYYLLKLIYQYVFLPNFSQGNSPAFRETVKAILRINKDEARKKMKKNKKFLRKYAFEQVREMEFKETPDQYMSYLQSEMREEKVRKAEKNDKGFEKNITMNFEKLLMQIFVKGFDVFLTTFAGKELLLSSEEKVIKETEISLSKKINEREKTLKASIQVEHQLVATNSAISYWLFCKLLDSRHLNELRNEMIKFKQSRIKFNHTQHAELIQNLLPIVELTILSNDYDEKNDSQNVDVSAYFEDKSLYETAPYVQTDDRTRVSFRPILKLEKYHTKSLIEALLKDNPQFRVAATDIQEWMHKREEIGELVEKRKNLHTEWAEGQQTLGAEKREEYRDYCKKIDRFNWKANKVTLTYLSQLHYLITDLLGRMVGFSALFERDLVYFSRSFSELGGETYHISDYKNLSGVLRLNAEVKPIKIKNIKVIDNEENPYKGNEPEVKPFLDRLHAYLENVIGIKAVHGKIRNQTAHLSVLQLELSMIESMNNLRDLMAYDRKLKNAVTKSMIKILDKHG

SEQ ID NO: 37 - DNA, 36 bp, synthetic construct
  misc_feature 1..36: note = CgaCas13a direct repeat
  source 1..36: mol_type = other DNA
attaaagactacctctaaatgtaagaggactataac

SEQ ID NO: 38 - DNA, 3669 bp, synthetic construct
  misc_feature 1..3669: note = Cga2Cas13a nucleic acid sequence
  source 1..3669: mol_type = other DNA
atgatcctgaagctgaagatcgacgagaaccacaagaatttcgagatcgagagcctgatcccaaaggagatcatccacctgaaggataaggccatcaagaccaaccaggtgtccgaggagtactgccagctggtgctggccctgctgaccacaaaccccggcaatcagctgaacatgaggatgacaaaggtgaagatcaacggcagccccgtgagcatgaatcgcagcaagctgaacggccacctggtgtggaatggcaccacaaataccgtgaacatcctgacaaagaaggagcagagcttcgccgcctcctttctgaacaagaccctggtgaaggccgaccaggtgaagggctacaaggtgctggccgagaatatcttcatcatctttgagcagctggagaagtccaactctgagaagcccagcgtgtacctgaacaatatccggagactgaaggaggccggcctgaagcggttctttaagagcaagtaccacgaggagatcaagtatacctctgagaagaatcagagcgtgccaacaaagctgaatctgatccccctgttctttaacgccgtggacagaatccaggaggacaagttcgatgagaagaactggtcctacttttgcaaggagatgtctccctacctggattataagaagagctatctgaataggaagaaggagatcctggccaattctatccagcagaacaggggcttcagcatgccaaccgcagaggagcctaacctgctgtctaagcggaagcagctgttccagcagtgggccatgaagtttcaggagagccctctgatccagcagaacaattttgccgtggagcagttcaataaggagtttgccaataagatcaacgagctggccgccgtgtataacgtggacgagctgtgcaccgccatcacagagaagctgatgaacttcgacaaggataagtccaataagaccagaaactttgagatcaagaagctgtggaagcagcaccctcacaacaaggataaggccctgatcaagctgttcaatcaggagggcaacgaggccctgaatcagtttaacatcgagctgggcaagtacttcgagcactattttcctaagacaggcaagaaggagagcgccgagtcctactatctgaatccacagaccatcatcaagacagtgggctaccagctgaggaacgccttcgtgcagtatctgctgcaagtgggcaagctgcaccagtacaacaagggcgtgctggacagccagaccctgcaggagatcggcatgtatgagggcttccagacaaagtttatggatgcctgcgtgttcgccagctcctctctgcggaatatcatccaggccaccacaaacgaggacatcctgaccagagagaagtttaagaaggagctggagaagaacgtggagctgaagcacgacctgttctttaagaccgagatcgtggaggagcgcgatgagaatcctgccaagaagatcgccatgacaccaaacgagctggacctgtgggcaatcaggggagccgtgcagagggtgcgcaatcagatcttccaccagcagatcaataagagacacgagcccaaccagctgaaggtcggctcctttgagaatggcgatctgggcaacgtgtcttaccagaaaaccatctatcagaagctgttcgacgccgagatcaaggatatcgagatctactttgccgagaagatcaagagctccggcgccctggagcagtattccatgaaggacctggagaagctgttcagcaacaaggagctgacactgtccctgggaggacaggtggtggccttcgccccttcttacaagaagctgtataagcagggctacttttatcagaatgagaaaaccatcgagctggagcagtttacagactacgatttctctaatgacgtgttcaaggccaactactatctgatcaagctgatctaccactacgtgttcctgccacagtttagccaggccaacaataagctgttcaaggacaccgtgcactacgtgatccagcagaataaggagctgaacaccacagagaaggataagaagaacaataagaagatcagaaagtatgcctttgagcaggtgaagctgatgaagaacgagtccccagagaagtacatgcagtatctgcagagggagatgcaggaggagcgcaccatcaaggaggccaagaaaaccaacgaggagaagcccaactacaatttcgagaagctgctgatccagatctttatcaagggcttcgacacctttctgaggaatttcgatctgaacctgaatcctgccgaggagctggtgggcacagtgaaggagaaggccgagggcctgcggaagagaaaggagcgcatcgccaagatcctgaacgtggacgagcagatcaagaccggcgatgaggagatcgccttctggatctttgcaaagctgctggacgcaaggcacctgagcgagctgagaaacgagatgatcaagtttaagcagtctagcgtgaagaagggcctgatcaagaatggcgatctgatcgagcagatgcagccaatcctggagctgtgcatcctgagcaacgacagcgagtccatggagaaggagtccttcgataagatcgaggtgtttctggagaaggtggagctggccaagaatgagccatacatgcaggaggacaagctgacccccgtgaagttcaggtttatgaagcagctggagaagtatcagacacgcaatttcatcgagaacctggtcatcgagaatccagagtttaaggtgtccgagaagatcgtgctgaactggcacgaggagaaggagaagatcgccgacctggtggataagcggaccaagctgcacgaggagtgggcctccaaggccagagagatcgaggagtacaatgagaagatcaagaagaacaagtctaagaagctggacaagcccgccgagttcgccaagtttgccgagtataagatcatctgtgaggccatcgagaacttcaataggctggatcacaaggtgcgcctgacatacctgaagaacctgcactatctgatgatcgacctgatgggcaggatggtgggcttctccgtgctgtttgagcgggatttcgtgtatatgggcagatcttatagcgccctgaagaagcagtctatctacctgaatgactatgataccttcgccaacatccgcgactgggaggtgaacgagaataagcacctgtttggcacatcctctagcgatctgaccttccaggagacagccgagtttaagaatctgaagaagcccatggagaaccagctgaaggccctgctgggcgtgaccaaccacagcttcgagatccggaacaatatcgcccacctgcacgtgctgagaaatgatggcaagggcgagggcgtgtctctgctgagctgcatgaacgacctgaggaagctgatgtcctacgatcgcaagctgaagaatgccgtgacaaaggccatcatcaagatcctggacaagcacggcatgatcctgaagctgaccaacaatgatcacacaaagcccttcgagattgagagcctgaagcctaagaagatcatccacctggagaagtccaaccactctttccctatggaccaggtgtcccaggagtactgtgatctggtgaagaagatgctggtgtttaccaattga

SEQ ID NO: 39 - protein, 1222 aa, synthetic construct
  REGION 1..1222: note = Cga2Cas13a amino acid sequence
MILKLKIDENHKNFEIESLIPKEIIHLKDKAIKTNQVSEEYCQLVLALLTTNPGNQLNMRMTKVKINGSPVSMNRSKLNGHLVWNGTTNTVNILTKKEQSFAASFLNKTLVKADQVKGYKVLAENIFIIFEQLEKSNSEKPSVYLNNIRRLKEAGLKRFFKSKYHEEIKYTSEKNQSVPTKLNLIPLFFNAVDRIQEDKFDEKNWSYFCKEMSPYLDYKKSYLNRKKEILANSIQQNRGFSMPTAEEPNLLSKRKQLFQQWAMKFQESPLIQQNNFAVEQFNKEFANKINELAAVYNVDELCTAITEKLMNFDKDKSNKTRNFEIKKLWKQHPHNKDKALIKLFNQEGNEALNQFNIELGKYFEHYFPKTGKKESAESYYLNPQTIIKTVGYQLRNAFVQYLLQVGKLHQYNKGVLDSQTLQEIGMYEGFQTKFMDACVFASSSLRNIIQATTNEDILTREKFKKELEKNVELKHDLFFKTEIVEERDENPAKKIAMTPNELDLWAIRGAVQRVRNQIFHQQINKRHEPNQLKVGSFENGDLGNVSYQKTIYQKLFDAEIKDIEIYFAEKIKSSGALEQYSMKDLEKLFSNKELTLSLGGQVVAFAPSYKKLYKQGYFYQNEKTIELEQFTDYDFSNDVFKANYYLIKLIYHYVFLPQFSQANNKLFKDTVHYVIQQNKELNTTEKDKKNNKKIRKYAFEQVKLMKNESPEKYMQYLQREMQEERTIKEAKKTNEEKPNYNFEKLLIQIFIKGFDTFLRNFDLNLNPAEELVGTVKEKAEGLRKRKERIAKILNVDEQIKTGDEEIAFWIFAKLLDARHLSELRNEMIKFKQSSVKKGLIKNGDLIEQMQPILELCILSNDSESMEKESFDKIEVFLEKVELAKNEPYMQEDKLTPVKFRFMKQLEKYQTRNFIENLVIENPEFKVSEKIVLNWHEEKEKIADLVDKRTKLHEEWASKAREIEEYNEKIKKNKSKKLDKPAEFAKFAEYKIICEAIENFNRLDHKVRLTYLKNLHYLMIDLMGRMVGFSVLFERDFVYMGRSYSALKKQSIYLNDYDTFANIRDWEVNENKHLFGTSSSDLTFQETAEFKNLKKPMENQLKALLGVTNHSFEIRNNIAHLHVLRNDGKGEGVSLLSCMNDLRKLMSYDRKLKNAVTKAIIKILDKHGMILKLTNNDHTKPFEIESLKPKKIIHLEKSNHSFPMDQVSQEYCDLVKKMLVFTN

SEQ ID NO: 40 - DNA, 36 bp, synthetic construct
  misc_feature 1..36: note = Cga2Cas13a direct repeat
  source 1..36: mol_type = other DNA
aatataaactacctctaaatgtaagaggactataac

SEQ ID NO: 41 - DNA, 3465 bp, synthetic construct
  misc_feature 1..3465: note = PprCas13a nucleic acid sequence
  source 1..3465: mol_type = other DNA
atgcgggtgtccaaggtgaaggtgaaggacggcggcaaggataagatggtgctggtgcacagaaagaccacaggcgcacagctggtgtactctggacagcccgtgtctaatgagacaagcaacatcctgcctgagaagaagaggcagtcctttgacctgtctaccctgaacaagacaatcatcaagttcgacacagccaagaagcagaagctgaatgtggatcagtacaagatcgtggagaagatctttaagtatcctaagcaggagctgccaaagcagatcaaggccgaggagatcctgccatttctgaatcacaagttccaggagcccgtgaagtactggaagaacggcaaggaggagtccttcaatctgaccctgctgatcgtggaggccgtgcaggcacaggacaagcgcaagctgcagccctactatgattggaaaacctggtatatccagacaaagagcgacctgctgaagaagtccatcgagaacaataggatcgatctgacagagaacctgtctaagcgcaagaaggccctgctggcctgggagacagagttcacagccagcggctccatcgacctgacccactaccacaaggtgtatatgacagacgtgctgtgcaagatgctgcaggatgtgaagccactgaccgacgataagggcaagatcaacacaaatgcctaccaccggggcctgaagaaggccctgcagaatcaccagcctgccatctttggcacccgggaggtgccaaacgaggccaatagagccgataaccagctgtccatctaccacctggaggtggtgaagtacctggagcactatttccccatcaagacctctaagcggagaaacacagccgacgatatcgcccactatctgaaggcccagaccctgaaaaccacaatcgagaagcagctggtgaacgccatcagagccaatatcatccagcagggcaagaccaaccaccacgagctgaaggccgacaccacaagcaatgatctgatccggatcaagacaaacgaggcctttgtgctgaatctgaccggcacatgtgccttcgccgccaacaatatcagaaatatggtggacaacgagcagaccaatgatatcctgggcaagggcgacttcatcaagtctctgctgaaggacaacacaaatagccagctgtactccttctttttcggcgagggcctgagcaccaataaggccgagaaggagacacagctgtggggcatcaggggagccgtgcagcagatccgcaacaatgtgaaccactataagaaggatgccctgaaaaccgtgttcaacatctccaatttcgagaaccccaccatcacagaccctaagcagcagaccaactacgccgatacaatctataaggccaggtttatcaatgagctggagaagatccctgaggccttcgcccagcagctgaaaaccggcggagccgtgtcttactatacaatcgagaatctgaagagcctgctgaccacatttcagttctctctgtgccgcagcaccatcccatttgcccccggcttcaagaaggtgtttaacggcggcatcaattaccagaacgccaagcaggacgagagcttctacgagctgatgctggagcagtatctgaggaaggagaactttgccgaggagtcctacaatgcccgctatttcatgctgaagctgatctataacaatctgttcctgccaggctttaccacagatcggaaggcctttgccgacagcgtgggcttcgtgcagatgcagaacaagaagcaggccgagaaagtgaatccaaggaagaaggaggcctacgcctttgaggccgtgcgccccatgaccgcagcagactccatcgccgattacatggcctatgtgcagtctgagctgatgcaggagcagaacaagaaggaggagaaggtggccgaggagacaaggatcaattttgagaagttcgtgctgcaggtgttcatcaagggctttgactccttcctgcgcgccaaggagtttgatttcgtgcagatgccacagcctcagctgaccgcaacagcctctaaccagcagaaggccgacaagctgaatcagctggaggccagcatcaccgccgattgcaagctgacaccccagtacgccaaggccgacgatgccacccacatcgccttctacgtgttctgcaagctgctggacgccgcccacctgagcaatctgcggaacgagctgatcaagttcagagagtccgtgaacgagtttaagttccaccacctgctggagatcatcgagatctgcctgctgagcgccgacgtggtgcccaccgactacagagatctgtatagctccgaggcagattgtctggcaaggctgcgcccttttatcgagcagggcgccgatatcacaaactggtccgacctgttcgtgcagtccgataagcactctcctgtgatccacgccaatatcgagctgtctgtgaagtacggcaccacaaagctgctggagcagatcatcaacaaggacacccagtttaagaccacagaggccaacttcaccgcctggaatacagcccagaagagcatcgagcagctgatcaagcagagggaggatcaccacgagcagtgggtgaaggccaagaacgccgacgataaggagaagcaggagcggaagagagagaagtccaatttcgcccagaagtttatcgagaagcacggcgacgattatctggacatctgcgattacatcaatacctataactggctggacaacaagatgcacttcgtgcacctgaatcgcctgcacggcctgacaatcgagctgctgggaaggatggcaggattcgtggccctgtttgacagagatttccagtttttcgacgagcagcagatcgccgatgagtttaagctgcacggcttcgtgaacctgcactccatcgacaagaagctgaatgaggtgcccaccaagaagatcaaggagatctacgatatccggaacaagatcatccagatcaacggcaataagatcaacgagtctgtgcgggccaatctgatccagtttatctctagcaagagaaactactataacaatgccttcctgcacgtgagcaatgacgagatcaaggagaagcagatgtacgatatcagaaaccacatcgcccactttaattatctgaccaaggacgccgccgatttcagcctgatcgacctgatcaacgagctgagggagctgctgcactacgatcgcaagctgaagaatgccgtgtccaaggcctttatcgacctgttcgataagcacggcatgatcctgaagctgaagctgaacgccgaccacaagctgaaggtggagtctctggagcctaagaagatctaccacctgggctcctctgccaaggataagccagagtaccagtattgtaccaaccaggtcatgatggcctattgcaatatgtgccggagcctgctggagatgaagaagtga

SEQ ID NO: 42 - protein, 1154 aa, synthetic construct
  REGION 1..1154: note = PprCas13a amino acid sequence
MRVSKVKVKDGGKDKMVLVHRKTTGAQLVYSGQPVSNETSNILPEKKRQSFDLSTLNKTIIKFDTAKKQKLNVDQYKIVEKIFKYPKQELPKQIKAEEILPFLNHKFQEPVKYWKNGKEESFNLTLLIVEAVQAQDKRKLQPYYDWKTWYIQTKSDLLKKSIENNRIDLTENLSKRKKALLAWETEFTASGSIDLTHYHKVYMTDVLCKMLQDVKPLTDDKGKINTNAYHRGLKKALQNHQPAIFGTREVPNEANRADNQLSIYHLEVVKYLEHYFPIKTSKRRNTADDIAHYLKAQTLKTTIEKQLVNAIRANIIQQGKTNHHELKADTTSNDLIRIKTNEAFVLNLTGTCAFAANNIRNMVDNEQTNDILGKGDFIKSLLKDNTNSQLYSFFFGEGLSTNKAEKETQLWGIRGAVQQIRNNVNHYKKDALKTVFNISNFENPTITDPKQQTNYADTIYKARFINELEKIPEAFAQQLKTGGAVSYYTIENLKSLLTTFQFSLCRSTIPFAPGFKKVFNGGINYQNAKQDESFYELMLEQYLRKENFAEESYNARYFMLKLIYNNLFLPGFTTDRKAFADSVGFVQMQNKKQAEKVNPRKKEAYAFEAVRPMTAADSIADYMAYVQSELMQEQNKKEEKVAEETRINFEKFVLQVFIKGFDSFLRAKEFDFVQMPQPQLTATASNQQKADKLNQLEASITADCKLTPQYAKADDATHIAFYVFCKLLDAAHLSNLRNELIKFRESVNEFKFHHLLEIIEICLLSADVVPTDYRDLYSSEADCLARLRPFIEQGADITNWSDLFVQSDKHSPVIHANIELSVKYGTTKLLEQIINKDTQFKTTEANFTAWNTAQKSIEQLIKQREDHHEQWVKAKNADDKEKQERKREKSNFAQKFIEKHGDDYLDICDYINTYNWLDNKMHFVHLNRLHGLTIELLGRMAGFVALFDRDFQFFDEQQIADEFKLHGFVNLHSIDKKLNEVPTKKIKEIYDIRNKIIQINGNKINESVRANLIQFISSKRNYYNNAFLHVSNDEIKEKQMYDIRNHIAHFNYLTKDAADFSLIDLINELRELLHYDRKLKNAVSKAFIDLFDKHGMILKLKLNADHKLKVESLEPKKIYHLGSSAKDKPEYQYCTNQVMMAYCNMCRSLLEMKK

SEQ ID NO: 43 - DNA, 36 bp, synthetic construct
  misc_feature 1..36: note = PprCas13a direct repeat
  source 1..36: mol_type = other DNA
cttgtggattatcccaaaattgaagggaactacaac

SEQ ID NO: 44 - DNA, 2913 bp, synthetic construct
  misc_feature 1..2913: note = LweCas13a nucleic acid sequence
  source 1..2913: mol_type = other DNA
atgctggccctgctgcaccaggaggtgccttcccagaagctgcacaacctgaagagcctgaataccgagtccctgacaaagctgtttaagccaaagttccagaacatgatcagctatccaccttccaagggagcagagcacgtgcagttctgcctgaccgacatcgccgtgcccgcaatcagggacctggatgagatcaagcctgactggggcatcttctttgagaagctgaagccatacacagattgggccgagtcttacatccactataagcagaccacaatccagaagagcatcgagcagaacaagatccagagcccagactccccaaggaagctggtgctgcagaagtatgtgaccgcctttctgaatggagagccactgggcctggacctggtggccaagaagtacaagctggccgatctggccgagtccttcaaggtggtggacctgaacgaggataagtctgccaattataagatcaaggcctgtctgcagcagcaccagcggaacatcctggacgagctgaaggaggaccccgagctgaatcagtacggcatcgaggtgaagaagtacatccagcggtatttcccaatcaagagggcaccaaaccgcagcaagcacgccagagccgactttctgaagaaggagctgatcgagtccaccgtggagcagcagttcaagaatgccgtgtaccactatgtgctggagcagggcaagatggaggcctacgagctgaccgatcccaagacaaaggacctgcaggatatcagatctggcgaggccttcagctttaagttcatcaacgcctgcgcctttgccagcaacaatctgaagatgatcctgaatcctgagtgtgagaaggacatcctgggcaagggcgatttcaagaagaacctgccaaattctaccacacagagcgacgtggtgaagaagatgatccccttcttttccgacgagatccagaacgtgaatttcgatgaggccatctgggccatccggggctctatccagcagatcagaaacgaggtgtatcactgcaagaagcactcttggaagagcatcctgaagatcaagggctttgagttcgagccaaacaatatgaagtacaccgactccgatatgcagaagctgatggacaaggatatcgccaagatccccgactttatcgaggagaagctgaagagctccggcatcatcaggttctactcccacgataagctgcagtctatctgggagatgaagcagggctttagcctgctgaccacaaacgccccttttgtgccatccttcaagagggtgtacgccaagggccacgactatcagacaagcaagaatcgctactatgacctgggcctgaccacattcgatatcctggagtacggagaggaggacttccgggcacgctatttcctgaccaagctggtgtactatcagcagtttatgccttggttcacagccgacaacaatgcctttcgcgatgccgccaacttcgtgctgaggctgaacaagaatcgccagcaggacgccaaggcctttatcaatatccgggaggtggaggagggcgagatgcctagagattacatgggctatgtgcagggccagatcgccatccacgaggactccaccgaggatacaccaaaccactttgagaagttcatctctcaggtgtttatcaagggcttcgactcccacatgcggtctgccgatctgaagttcatcaagaaccccagaaatcagggcctggagcagagcgagatcgaggagatgtcctttgacatcaaggtggagcctagcttcctgaagaacaaggacgattatatcgccttttggaccttctgcaagatgctggacgcaaggcacctgtccgagctgagaaatgagatgatcaagtacgatggccacctgacaggcgagcaggagatcatcggcctggccctgctgggagtggactctagggagaacgattggaagcagttcttttctagcgagcgcgagtacgagaagatcatgaagggctatgtgggcgaggagctgtaccagcgggagccttatagacagagcgacggcaagaccccaatcctgttcaggggagtggagcaggcaaggaagtacggcaccgagacagtgatccagcggctgtttgatgcctctcctgagttcaaggtgagcaagtgtaacatcacagagtgggagagacagaaggagacaatcgaggagacaatcgagcggagaaaggagctgcacaacgagtgggagaagaatcccaagaagcctcagaacaatgccttctttaaggagtacaaggagtgctgtgacgccatcgatgcctataactggcacaagaataagaccacactggtgtacgtgaatgagctgcaccacctgctgatcgagatcctgggcaggtacgtgggctatgtggccatcgccgaccgcgattttcagtgcatggccaaccagtatttcaagcactctggcatcaccgagagggtggagtactggggcgacaatcgcctgaagagcatcaagaagctggatacatttctgaagaaggagggcctgttcgtgtccgagaagaacgccaggaatcacatcgcccacctgaactacctgtccctgaagtctgagtgtaccctgctgtatctgtccgagcggctgagagagatctttaagtacgacaggaagctgaagaatgccgtgtccaagtctctgatcgacatcctggatcgccacggcatgtctgtggtgttcgccaacctgaaggagaataagcaccggctggtcatcaagagcctggagccaaagaagctgagacacctgggcgagaagaagatcgataacggctacatcgagacaaatcaggtgagcgaggagtattgtggcatcgtgaagagactgctggagatctga

SEQ ID NO: 45 - protein, 970 aa, synthetic construct
  REGION 1..970: note = LweCas13a amino acid sequence
MLALLHQEVPSQKLHNLKSLNTESLTKLFKPKFQNMISYPPSKGAEHVQFCLTDIAVPAIRDLDEIKPDWGIFFEKLKPYTDWAESYIHYKQTTIQKSIEQNKIQSPDSPRKLVLQKYVTAFLNGEPLGLDLVAKKYKLADLAESFKVVDLNEDKSANYKIKACLQQHQRNILDELKEDPELNQYGIEVKKYIQRYFPIKRAPNRSKHARADFLKKELIESTVEQQFKNAVYHYVLEQGKMEAYELTDPKTKDLQDIRSGEAFSFKFINACAFASNNLKMILNPECEKDILGKGDFKKNLPNSTTQSDVVKKMIPFFSDEIQNVNFDEAIWAIRGSIQQIRNEVYHCKKHSWKSILKIKGFEFEPNNMKYTDSDMQKLMDKDIAKIPDFIEEKLKSSGIIRFYSHDKLQSIWEMKQGFSLLTTNAPFVPSFKRVYAKGHDYQTSKNRYYDLGLTTFDILEYGEEDFRARYFLTKLVYYQQFMPWFTADNNAFRDAANFVLRLNKNRQQDAKAFINIREVEEGEMPRDYMGYVQGQIAIHEDSTEDTPNHFEKFISQVFIKGFDSHMRSADLKFIKNPRNQGLEQSEIEEMSFDIKVEPSFLKNKDDYIAFWTFCKMLDARHLSELRNEMIKYDGHLTGEQEIIGLALLGVDSRENDWKQFFSSEREYEKIMKGYVGEELYQREPYRQSDGKTPILFRGVEQARKYGTETVIQRLFDASPEFKVSKCNITEWERQKETIEETIERRKELHNEWEKNPKKPQNNAFFKEYKECCDAIDAYNWHKNKTTLVYVNELHHLLIEILGRYVGYVAIADRDFQCMANQYFKHSGITERVEYWGDNRLKSIKKLDTFLKKEGLFVSEKNARNHIAHLNYLSLKSECTLLYLSERLREIFKYDRKLKNAVSKSLIDILDRHGMSVVFANLKENKHRLVIKSLEPKKLRHLGEKKIDNGYIETNQVSEEYCGIVKRLLEI

SEQ ID NO: 46 - DNA, 36 bp, synthetic construct
  misc_feature 1..36: note = LweCas13a direct repeat
  source 1..36: mol_type = other DNA
gatttagagtacctcaaaatagaagaggtctaaaac

SEQ ID NO: 47 - DNA, 3156 bp, synthetic construct
  misc_feature 1..3156: note = LbfCas13a nucleic acid sequence
  source 1..3156: mol_type = other DNA
atgaagatcaccaagatgcgggtggacggcagaaccatcgtgatggagcggacatccaaggagggccagctgggctatgagggcatcgacggcaacaagaccacagagatcatctttgataagaagaaggagagcttctacaagtccatcctgaacaagaccgtgagaaagcctgatgagaaggagaagaatcggagaaagcaggccatcaacaaggccatcaataaggagatcacagagctgatgctggccgtgctgcaccaggaggtgcctagccagaagctgcacaacctgaagagcctgaataccgagtccctgacaaagctgtttaagccaaagttccagaatatgatctcttatccacctagcaagggagcagagcacgtgcagttctgcctgaccgacatcgccgtgcccgcaatccgggacctggatgagatcaagcctgactggggcatcttctttgagaagctgaagccatacacagattgggccgagtcctacatccactataagcagaccacaatccagaagtctatcgagcagaacaagatccagagcccagactcccccagaaagctggtgctgcagaagtatgtgaccgcctttctgaatggagagccactgggcctggacctggtggccaagaagtacaagctggccgatctggccgagtctttcaagctggtggacctgaacgaggataagagcgccaattataagatcaaggcctgtctgcagcagcaccagcggaacatcctggacgagctgaaggaggaccccgagctgaatcagtacggcatcgaggtgaagaagtacatccagaggtatttcccaatcaagagggcccccaaccgctctaagcacgccagagccgactttctgaagaaggagctgatcgagagcaccgtggagcagcagttcaagaatgccgtgtaccactatgtgctggagcagggcaagatggaggcctacgagctgaccgatcccaagacaaaggacctgcaggatatcaggtctggcgaggccttcagctttaagttcatcaacgcctgcgcctttgcctccaacaatctgaagatgatcctgaaccctgagtgtgagaaggacatcctgggcaagggcaatttcaagaagaacctgccaaattccaccacacgctctgatgtggtgaagaagatgatccccttctttagcgacgagctgcagaacgtgaatttcgatgaggccatctgggccatccggggctccatccagcagatcagaaatgaggtgtatcactgcaagaagcactcttggaagagcatcctgaagatcaagggctttgagttcgagccaaacaatatgaagtacgccgacagcgatatgcagaagctgatggacaaggatatcgccaagatccccgagtttatcgaggagaagctgaagagctccggcgtggtgcggttctacagacacgacgagctgcagagcatctgggagatgaagcagggcttttccctgctgaccacaaacgccccttttgtgcccagcttcaagcgggtgtacgccaagggccacgactatcagacctccaagaacagatactataatctggacctgaccacattcgatatcctggagtacggcgaggaggattttcgggccagatatttcctgaccaagctggtgtactatcagcagtttatgccctggttcacagccgacaacaatgcctttagggatgccgccaacttcgtgctgaggctgaacaagaatcgccagcaggacgccaaggcctttatcaatatccgggaggtggaggagggcgagatgcctagagattacatgggctatgtgcagggccagatcgccatccacgaggacagcatcgaggataccccaaaccactttgagaagttcatctcccaggtgtttatcaagggcttcgacaggcacatgcgctctgccaatctgaagttcatcaagaacccccgcaatcagggcctggagcagtccgagatcgaggagatgtcttttgatatcaaggtggagccttctttcctgaagaacaaggacgattatatcgccttttggatcttctgcaagatgctggacgcaaggcacctgagcgagctgagaaatgagatgatcaagtacgatggccacctgaccggcgagcaggagatcatcggcctgrccctgctgggagtggactcccgggagaacgattggaagcagttcttttctagcgagagagagtacgagaagatcatgaagggctatgtggtggaggagctgtaccagcgggagccttatagacagtctgacggcaagacaccaatcctgttcaggggagtggagcaggcaaggaagtacggcaccgagacagtgatccagaggctgtttgatgccaaccccgagttcaaggtgagcaagtgtaatctggccgagtgggagcgccagaaggagacaatcgaggagacaatcaagaggcgcaaggagctgcacaacgagtgggccaagaatcccaagaagcctcagaacaatgccttctttaaggagtacaaggagtgctgtgacgccatcgatgcctataactggcacaagaataagaccacactggcctacgtgaacgagctgcaccacctgctgatcgagatcctgggcaggtacgtgggctatgtggccatcgccgaccgcgattttcagtgcatggccaaccagtatttcaagcactccggcatcaccgagagggtggagtactggggcgacaatcgcctgaagtctatcaagaagctggatacatttctgaagaaggagggcctgttcgtgagcgagaagaacgcccggaatcacatcgcccacctgaactacctgtccctgaagtctgagtgtaccctgctgtatctgtccgagaggctgcgcgagatctttaagtacgaccggaagctgaagaatgccgtgtccaagtctctgatcgacatcctggatagacacggcatgtccgtggtgttcgccaacctgaaggagaataagcaccggctggtcatcaagagcctggagcctaagaagctgcgccacctgggcggcaagaagatcgatggcggctacatcgagacaaaccaggtgagcgaggagtattgtggcatcgtgaagagactgctggagatgtga

SEQ ID NO: 48 - protein, 1050 aa, synthetic construct
  REGION 1..1050: note = LbfCas13a amino acid sequence
MKITKMRVDGRTIVMERTSKEGQLGYEGIDGNKTTEIIFDKKKESFYKSILNKTVRKPDEKEKNRRKQAINKAINKEITELMLAVLHQEVPSQKLHNLKSLNTESLTKLFKPKFQNMISYPPSKGAEHVQFCLTDIAVPAIRDLDEIKPDWGIFFEKLKPYTDWAESYIHYKQTTIQKSIEQNKIQSPDSPRKLVLQKYVTAFLNGEPLGLDLVAKKYKLADLAESFKLVDLNEDKSANYKIKACLQQHQRNILDELKEDPELNQYGIEVKKYIQRYFPIKRAPNRSKHARADFLKKELIESTVEQQFKNAVYHYVLEQGKMEAYELTDPKTKDLQDIRSGEAFSFKFINACAFASNNLKMILNPECEKDILGKGNFKKNLPNSTTRSDVVKKMIPFFSDELQNVNFDEAIWAIRGSIQQIRNEVYHCKKHSWKSILKIKGFEFEPNNMKYADSDMQKLMDKDIAKIPEFIEEKLKSSGVVRFYRHDELQSIWEMKQGFSLLTTNAPFVPSFKRVYAKGHDYQTSKNRYYNLDLTTFDILEYGEEDFRARYFLTKLVYYQQFMPWFTADNNAFRDAANFVLRLNKNRQQDAKAFINIREVEEGEMPRDYMGYVQGQIAIHEDSIEDTPNHFEKFISQVFIKGFDRHMRSANLKFIKNPRNQGLEQSEIEEMSFDIKVEPSFLKNKDDYIAFWIFCKMLDARHLSELRNEMIKYDGHLTGEQEIIGLLLGVDSRENDWKQFFSSEREYEKIMKGYVVEELYQREPYRQSDGKTPILFRGVEQARKYGTETVIQRLFDANPEFKVSKCNLAEWERQKETIEETIKRRKELHNEWAKNPKKPQNNAFFKEYKECCDAIDAYNWHKNKTTLAYVNELHHLLIEILGRYVGYVAIADRDFQCMANQYFKHSGITERVEYWGDNRLKSIKKLDTFLKKEGLFVSEKNARNHIAHLNYLSLKSECTLLYLSERLREIFKYDRKLKNAVSKSLIDILDRHGMSVVFANLKENKHRLVIKSLEPKKLRHLGGKKIDGGYIETNQVSEEYCGIVKRLLEM

SEQ ID NO: 49 - DNA, 36 bp, synthetic construct
  misc_feature 1..36: note = LbfCas13a direct repeat
  source 1..36: mol_type = other DNA
gatttagagtacctcaaaacaaaagaggactaaaac

SEQ ID NO: 50 - DNA, 3459 bp, synthetic construct
  misc_feature 1..3459: note = Lwa2Cas13a nucleic acid sequence
  source 1..3459: mol_type = other DNA
atgaaggtgaccaaggtggacggcatcagccacaagaagtacatcgaggagggcaagctggtgaagtccacctctgaggagaaccggacatctgagagactgagcgagctgctgtccatcaggctggacatctacatcaagaaccccgataatgcctccgaggaggagaaccgcatccggagagagaatctgaagaagttcttttctaacaaggtgctgcacctgaaggacagcgtgctgtacctgaagaacaggaaggagaagaatgccgtgcaggataagaattattccgaggaggacatctctgagtacgatctgaagaacaagaatagcttctccgtgctgaagaagatcctgctgaacgaggacgtgaatagcgaggagctggagatcttcaggaaggatgtggaggccaagctgaacaagatcaattctctgaagtatagctttgaggagaacaaggccaattaccagaagatcaacgagaacaatgtggagaaagtgggcggcaagagcaagcgcaacatcatctatgactactatcgggagtccgccaagagaaatgattacatcaacaatgtgcaggaggccttcgacaagctgtataagaaggaggatatcgagaagctgttctttctgatcgagaactccaagaagcacgagaagtacaagatccgggagtactatcacaagatcatcggcagaaagaacgacaaggagaatttcgccaagatcatctatgaggagatccagaacgtgaacaatatcaaggagctgatcgagaagatccctgatatgagcgagctgaagaagtcccaggtgttttacaagtactatctggacaaggaggagctgaacgataagaatatcaagtatgccttctgccactttgtggagatcgagatgagccagctgctgaagaactacgtgtataagcggctgtctaacatcagcaatgacaagatcaagagaatcttcgagtaccagaatctgaagaagctgatcgagaacaagctgctgaataagctggacacctatgtgcgcaactgtggcaagtacaattactatctgcaagtgggcgagatcgccacatccgatttcatcgccaggaaccgccagaatgaggcctttctgcggaacatcatcggcgtgagctccgtggcctacttttctctgagaaatatcctggagacagagaacgagaatgacatcacaggccggatgagaggcaagaccgtgaagaacaataagggcgaggagaagtacgtgagcggcgaggtggataagatctacaacgagaataagcagaacgaggtgaaggagaatctgaagatgttctacagctatgactttaacatggataacaagaatgagatcgaggacttctttgccaatatcgatgaggccatctctagcatccggcacggcatcgtgcacttcaacctggagctggagggcaaggacatcttcgcctttaagaatatcgccccatccgagatctctaagaagatgtttcagaacgagatcaatgagaagaagctgaagctgaagatcttcaagcagctgaactccgccaacgtgttcaactactatgagaaggacgtgatcatcaagtacctgaagaacaccaagttcaattttgtgaacaagaatatccccttcgtgccttcctttacaaagctgtataacaagatcgaggatctgagaaataccctgaagttcttttggtctgtgccaaaggacaaggaggagaaggatgcccagatctacctgctgaagaacatctactatggcgagttcctgaacaagtttgtgaagaacagcaaggtgttctttaagatcacaaatgaagtgatcaagatcaacaagcagcggaatcagaaaaccggccactacaagtatcagaagttcgagaacatcgagaaaaccgtgcccgtggagtacctggccatcatccagagcagagagatgatcaacaatcaggacaaggaggagaagaacacctatatcgatttcatccagcagatcttcctgaagggctttatcgactatctgaataagaacaatctgaagtacatcgagtccaacaacaacaacgacaacaacgatatcttttctaagatcaagatcaagaaggacaacaaggagaagtatgataagatcctgaagaattacgagaagcacaacaggaataaggagatcccccacgagatcaacgagttcgtgcgcgagatcaagctgggcaagatcctgaagtatacagagaacctgaatatgttttacctgatcctgaagctgctgaaccacaaggagctgaccaatctgaagggctctctggagaagtaccagagcgccaacaaggaggagacattcagcgatgagctggagctgatcaatctgctgaacctggacaacaatcgggtgaccgaggattttgagctggaggccaacgagatcggcaagttcctggactttaacgagaataagatcaaggataggaaggagctgaagaagttcgacacaaacaagatctactttgatggcgagaatatcatcaagcaccgcgccttctataacatcaagaagtacggcatgctgaatctgctggagaagatcgccgacaaggccaagtataagatcagcctgaaggagctgaaggagtactccaataagaagaacgagatcgagaagaactataccatgcagcagaatctgcacaggaagtacgcccgccctaagaaggatgagaagttcaacgacgaggattacaaggagtatgagaaggccatcggcaacatccagaagtacacccacctgaagaataaggtggagtttaatgagctgaacctgctgcagggcctgctgctgaagatcctgcacaggctggtgggctatacatccatctgggagcgcgacctgaggttccgcctgaagggcgagtttccagagaaccactacatcgaggagatcttcaatttcgataactccaagaatgtgaagtacaagtctggccagatcgtggagaagtacatcaacttctataaggagctgtataaggacaatgtggagaagcggagcatctactccgataagaaggtgaagaagctgaagcaggagaagaaggacctgtatatcagaaactacatcgcccactttaattatatccctcacgccgagatcagcctgctggaggtgctggagaacctgaggaagctgctgtcttacgaccgcaagctgaagaatgccatcatgaagagcatcgtggatatcctgaaggagtatggcttcgtggccaccttcaagatcggcgccgacaagaagatcgagatccagaccctggagagcgagaagatcgtgcacctgaagaacctgaagaagaagaagctgatgacagatcggaacagcgaggagctgtgcgagctggtgaaagtgatgttcgagtacaaggccctggagtga

SEQ ID NO: 51 - protein, 1152 aa, synthetic construct
  REGION 1..1152: note = Lwa2Cas13a amino acid sequence
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 52 - DNA, 36 bp, synthetic construct
  misc_feature 1..36: note = Lwa2Cas13a direct repeat
  source 1..36: mol_type = other DNA
gatatagataaccccaaaaacgaagggatctaaaac

SEQ ID NO: 53 - DNA, 3858 bp, synthetic construct
  misc_feature 1..3858: note = RcsCas13a nucleic acid sequence
  source 1..3858: mol_type = other DNA
atgcagattggaaaagtgcagggaaggactattagcgagtttggggaccccgcaggaggactgaagagaaagattagcaccgatggaaagaatcgcaaggaactgccagcacacctgtcaagtgatccaaaggccctgattggacagtggatctcagggatcgacaaaatctaccggaagcccgatagtaggaaatccgatggcaaagcaatccactccccaacccctagcaaaatgcagtttgacgcaagggatgacctgggagaggcattctggaaactggtgtcagaagctggcctggctcaggatagtgactatgatcagtttaagcggcgactgcatccatacggagacaaatttcagcctgctgatagcggagcaaaactgaagttcgaagccgacccacctgagcctcaggccttccacggcaggtggtatggagcaatgtccaagcgcgggaatgacgcaaaggagctggcagtcgctctgtacgaacatctgcacgtcgatgagaaaagaatcgatggccagcctaagcggaatcctaagactgataaattcgctccagggctggtggtggcccgcgctctggggattgagtcaagcgtgctgccaagaggaatggctagactggctaggaattggggcgaagaggaaatccagacttactttgtcgtggatgtggccgcatcagtgaaggaggtggctaaagctgctgtgagcgcagcacaggctttcgatccacctcgccaggtcagcgggagaagtctgagtcccaaagtggggttcgcactggcagagcatctggaaagggtgacaggctccaagaggtgctcattcgatcccgcagccggcccttctgtcctggcactgcacgacgaagtgaagaaaacttataaaagactgtgcgcccggggaaagaatgcagccagagcttttccagccgacaagaccgaactgctggctctgatgagacatacccatgagaacagagtccggaatcagatggtcagaatgggaagggtgtcagaataccgaggccagcaggccggggatctggctcagtcccactattggacaagtgccggccagacagaaatcaaagaaagcgagatctttgtcaggctgtgggtgggagcttttgcactggctgggcgcagcatgaaagcatggattgacccaatggggaagatcgtgaacactgagaagaatgatagagatctgactgccgccgtcaacatcagacaggtaatctccaacaaggagatggtcgctgaggcaatggctcggcgcggaatctatttcggcgagacaccagagctggatagactgggggcagagggaaatgaaggcttcgtgttcgctctgctgcgatacctgagagggtgtagaaaccagacttttcatctgggggctcgggcaggatttctgaaggaaatccgcaaggagctggaaaagacacggtggggcaaggcaaaagaagccgagcatgtggtcctgaccgacaagacagtggcagccattagagcaatcattgacaatgacgccaaagccctgggcgcccggctgctggcagacctgagcggagcttttgtggcacactatgcaagcaaagaacacttcagcacactgtattccgagatcgtcaaagcagtgaaagacgctcctgaagtgtcatccgggctgcctcggctgaagctgctgctgaaacgggcagacggagtcaggggatacgtgcatggactgcgggatacccgaaaacatgctttcgccactaagctgccacctcctccagctccacgggaactggacgaccctgcaacaaaggccaggtatatcgctctgctgcggctgtatgatggcccatttcgggcctacgcaagtggaatcactggaactgccctggcaggacctgctgccagagctaaagaggcagctactgcactggcccagtccgtgaatgtcactaaagcatattccgatgtgatggagggacgcacctcacgactgaggccacctaatgatggcgaaaccctgagggaatacctgagtgcactgactggcgagactgccacagagttccgggtgcagattggctatgagtccgactcagagaatgctagaaagcaggccgagtttatcgagaactataggcgagatatgctggccttcatgttcgaggattacattcgagcaaaggggttcgactggattctgaagattgaacctggggccacagcaatgaccagagcccctgtgctgccagagccaatcgatacacgaggccagtatgaacactggcaggccgcactgtacctggtcatgcacttcgtcccagctagtgatgtgagcaacctgctgcaccagctgagaaagtgggaagcactgcagggcaaatacgagctggtccaggatggggacgccacagaccaggcagatgcccggagggaggccctggatctggtgaaaaggttccgcgatgtgctggtcctgtttctgaaaacaggggaagccagatttgaaggccgcgccgctccatttgatctgaagcctttccgggcactgtttgctaatccagctactttcgatcggctgtttatggccacaccaaccactgctaggcccgctgaagatgaccccgagggcgacggagcctccgaaccagagctgcgggtcgccaggacactgcgcgggctgcgccagattgcacgatataaccacatggcagtgctgagcgatctgttcgctaaacataaagtgcgggatgaggaagtcgcacgcctggccgagattgaggacgaaactcaggaaaaatctcagattgtcgccgcacaggaactgaggacagatctgcacgataaagtcatgaaatgccaccctaaaaccatcagccccgaggagcggcagagttacgccgccgcaatcaaaaccattgaggagcacaggttcctggtgggcagagtgtacctgggggatcacctgcggctgcatcgcctgatgatggatgtcatcggccgcctgattgattacgctggcgcttatgagcgcgatactggcactttcctgatcaacgccagcaaacagctgggggctggcgctgattgggcagtgaccattgcaggcgccgctaataccgacgcacgcacccagacccggaaagacctggcacatttcaacgtgctggatagagccgacggaacccccgacctgacagctctggtgaacagggcaagagaaatgatggcctacgatcgcaaacgcaaaaacgcagtcccacggagtatcctggatatgctggctcggctggggctgacactgaagtggcagatgaaggaccacctgctgcaggacgctacaatcacccaggctgcaatcaaacacctggataaagtgcggctgaccgtggggggccccgccgccgtcaccgaagcacgcttctcacaggactacctgcagatggtcgctgcagtgttcaatgggagcgtgcagaaccctaaaccacggcgccgggatgatggggacgcctggcacaagccacccaagcctgcaactgctcagtctcagcctgatcagaaacctcccaacaaagcaccttctgcagggtccaggctgccacccccacaggtcggggaggtctacgaaggagtcgtggtcaaagtcattgataccggctcactgggctttctggccgtggagggggtggctggcaatattggcctgcatatctcccgcctgcgacgaatcagagaggacgccatcattgtcgggcggagatacagattcagggtcgaaatctatgtcccccccaagagcaacacttccaaactgaacgccgctgacctggtgcgaattgactga

SEQ ID NO: 54 - protein, 1285 aa, synthetic construct
  REGION 1..1285: note = RcsCas13a amino acid sequence
MQIGKVQGRTISEFGDPAGGLKRKISTDGKNRKELPAHLSSDPKALIGQWISGIDKIYRKPDSRKSDGKAIHSPTPSKMQFDARDDLGEAFWKLVSEAGLAQDSDYDQFKRRLHPYGDKFQPADSGAKLKFEADPPEPQAFHGRWYGAMSKRGNDAKELAVALYEHLHVDEKRIDGQPKRNPKTDKFAPGLVVARALGIESSVLPRGMARLARNWGEEEIQTYFVVDVAASVKEVAKAAVSAAQAFDPPRQVSGRSLSPKVGFALAEHLERVTGSKRCSFDPAAGPSVLALHDEVKKTYKRLCARGKNAARAFPADKTELLALMRHTHENRVRNQMVRMGRVSEYRGQQAGDLAQSHYWTSAGQTEIKESEIFVRLWVGAFALAGRSMKAWIDPMGKIVNTEKNDRDLTAAVNIRQVISNKEMVAEAMARRGIYFGETPELDRLGAEGNEGFVFALLRYLRGCRNQTFHLGARAGFLKEIRKELEKTRWGKAKEAEHVVLTDKTVAAIRAIIDNDAKALGARLLADLSGAFVAHYASKEHFSTLYSEIVKAVKDAPEVSSGLPRLKLLLKRADGVRGYVHGLRDTRKHAFATKLPPPPAPRELDDPATKARYIALLRLYDGPFRAYASGITGTALAGPAARAKEAATALAQSVNVTKAYSDVMEGRTSRLRPPNDGETLREYLSALTGETATEFRVQIGYESDSENARKQAEFIENYRRDMLAFMFEDYIRAKGFDWILKIEPGATAMTRAPVLPEPIDTRGQYEHWQAALYLVMHFVPASDVSNLLHQLRKWEALQGKYELVQDGDATDQADARREALDLVKRFRDVLVLFLKTGEARFEGRAAPFDLKPFRALFANPATFDRLFMATPTTARPAEDDPEGDGASEPELRVARTLRGLRQIARYNHMAVLSDLFAKHKVRDEEVARLAEIEDETQEKSQIVAAQELRTDLHDKVMKCHPKTISPEERQSYAAAIKTIEEHRFLVGRVYLGDHLRLHRLMMDVIGRLIDYAGAYERDTGTFLINASKQLGAGADWAVTIAGAANTDARTQTRKDLAHFNVLDRADGTPDLTALVNRAREMMAYDRKRKNAVPRSILDMLARLGLTLKWQMKDHLLQDATITQAAIKHLDKVRLTVGGPAAVTEARFSQDYLQMVAAVFNGSVQNPKPRRRDDGDAWHKPPKPATAQSQPDQKPPNKAPSAGSRLPPPQVGEVYEGVVVKVIDTGSLGFLAVEGVAGNIGLHISRLRRIREDAIIVGRRYRFRVEIYVPPKSNTSKLNAADLVRID

SEQ ID NO: 55 - DNA, 36 bp, synthetic construct
  misc_feature 1..36: note = RcsCas13a direct repeat
  source 1..36: mol_type = other DNA
gcctcacatcaccgccaagacgacggcggactgaac

SEQ ID NO: 56 - DNA, 3858 bp, synthetic construct
  misc_feature 1..3858: note = RcrCas13a nucleic acid sequence
  source 1..3858: mol_type = other DNA
atgcagattgggaaagtccagggcagaaccattagcgagtttggcgaccccgcaggagggctgaaaaggaagattagcaccgacggaaagaaccggaaagaactgcccgcccatctgtcaagcgatccaaaggctctgatcggccagtggattagcggcattgataagatctacaggaagcctgattcaagaaagagcgatggaaaagccatccatagcccaactccaagcaagatgcagtttgatgccagggacgacctgggggaagcattctggaaactggtgtcagaggctgggctggctcaggacagcgattacgatcagtttaaaaggcggctgcatccctatggggataagtttcagcccgccgatagcggcgctaaactgaaattcgaagctgatcctccagagccccaggcttttcacggccgctggtatggggcaatgagcaaaagaggaaacgacgctaaagaactggcagcagcactgtatgaacatctgcatgtcgatgaaaaacgaattgatggccagccaaaaaggaacccaaagacagataaattcgcaccagggctggtcgtggccagggcactggggatcgagagctctgtgctgccacgggggatggcaagactggcacggaattggggggaagaggaaatccagacatacttcgtggtcgacgtggcagcaagcgtgaaggaagtggcaaaggcagccgtgagcgccgcacaggccttcgacccacctcggcaggtgtccggcaggtcactgagcccaaaggtcgggtttgccctggctgaacatctggagcgggtcacaggctccaaaaggtgcagcttcgatcctgcagctgggccaagcgtcctggccctgcacgacgaagtcaagaaaacctataaacggctgtgcgcccggggaaagaacgccgcaagagcctttccagcagacaaaacagaactgctggccctgatgcggcatacccacgagaaccgggtgaggaatcagatggtcagaatggggcgggtgagtgagtatagggggcagcaggcaggggacctggcacagagccactactggacaagcgcaggacagaccgaaatcaaggagtcagagatttttgtccgcctgtgggtcggggctttcgcactggccgggaggtcaatgaaagcctggattgatcccatgggaaagatcgtcaacactgaaaagaacgatcgcgacctgaccgctgcagtcaacattcggcaggtcatcagcaacaaggagatggtggccgaagctatggcacgacgggggatctactttggagaaaccccagagctggataggctgggagcagaaggcaatgagggcttcgtctttgcactgctgagatacctgagagggtgcaggaatcagacctttcacctgggggccagggcagggttcctgaaagagattagaaaagaactggaaaaaacccggtgggggaaggccaaggaggcagagcacgtggtcctgaccgataaaactgtcgctgccatcagagccatcatcgacaacgatgccaaggcactgggggcccggctgctggcagacctgtcaggggcttttgtcgctcactatgccagtaaagaacactttagcaccctgtactcagaaattgtcaaggctgtgaaggacgcaccagaggtgtcatccgggctgccccggctgaagctgctgctgaagagggcagacggagtcagaggctatgtccatgggctgagggatacaaggaaacacgcctttgctactaaactgccaccacctccagcaccaagggaactggatgacccagccaccaaagccagatacatcgcactgctgagactgtacgatggaccattcagggcttatgcatccggcattactgggacagctctggccgggccagctgcacgagccaaggaggccgcaaccgctctggcacagtccgtgaatgtcacaaaagcatacagcgatgtgatggagggcaggtcaagcagactgaggccacccaacgatggggaaacactgagagaatacctgagcgcactgacaggagaaacagcaacagaatttcgggtccagattggctatgaaagcgactcagagaatgctcggaagcaggccgagttcattgagaactatagacgggatatgctggcttttatgtttgaggactacatccgggctaaggggttcgactggattctgaaaatcgagcccggagcaactgcaatgacacgggcacctgtgctgcccgagccaattgacacccgggggcagtacgaacactggcaggcagcactgtatctggtaatgcatttcgtgcccgcatcagatgtcagcaacctgctgcatcagctgaggaagtgggaggccctgcaggggaaatatgaactggtgcaggatggcgacgcaacagatcaggcagacgcacgacgagaggccctggatctggtcaagcgatttcgcgatgtcctggtgctgttcctgaaaacaggagaggcccggttcgagggacgggcagctccattcgacctgaaacccttccgagcactgttcgcaaatccagcaactttcgatcggctgtttatggctacacctaccacagcaaggccagcagaggatgatcccgagggggacggcgcatcagaaccagagctgagggtggcccggactctgagagggctgcggcagattgccaggtataatcacatggcagtcctgagcgatctgtttgcaaagcacaaagtccgagatgaggaagtcgcaaggctggcagaaattgaagatgagactcaggagaaatcacagatcgtcgctgcacaggaactgagaaccgacctgcatgacaaagtcatgaagtgtcatcccaaaaccatctctcctgaagaaagacagtcctacgccgcagctattaagaccatcgaagagcataggtttctggtggggagggtctatctgggagatcatctgcggctgcacagactgatgatggatgtgattggacggctgatcgattacgctggagcctacgagagggatactgggacatttctgatcaatgcctcaaagcagctgggggccggcgcagactgggcagtgacaattgccggggcagcaaacactggcgccaggactcagactcggaaggacctggcccactttaacgtgctggatagggccgacgggacacctgacctgaccgccctggtgaatcgagcaagagaaatgatggcatacgatcggaaaagaaaaaacgcagtcccaagatcaattctggatatgctggccaggctgggactgactctgaaatggcagatgaaggatcacctgctgcaggatgcaactattacccaggctgcaattaagcatctggacaaagtgcggctgacagtgggcgggcctgcagctgtgactgaggcacgattctcccaggactacctgcagatggtggccgcagtgtttaacgggtctgtccagaatccaaagccaaggaggagggacgacggcgacgcatggcataaaccacctaagccagcaactgcacagtcccagcccgaccagaagccacccaacaaagcacctagcgcagggtcacggctgcccccaccacaggtcggggaagtctatgaaggggtggtggtgaaagtgatcgataccgggtcactgggattcctggcagtcgaaggggtcgcagggaatatcggcctgcatattagtaggctgaggagaatcagagaagatgctattatcgtgggcaggagatacaggttttgggtcgagatctacgtgccccccaagtctaatacctccaaactgaacgctgctgacctggtgcgaattgactga

SEQ ID NO: 57 - protein, 1285 aa, synthetic construct
  REGION 1..1285: note = RcrCas13a amino acid sequence
MQIGKVQGRTISEFGDPAGGLKRKISTDGKNRKELPAHLSSDPKALIGQWISGIDKIYRKPDSRKSDGKAIHSPTPSKMQFDARDDLGEAFWKLVSEAGLAQDSDYDQFKRRLHPYGDKFQPADSGAKLKFEADPPEPQAFHGRWYGAMSKRGNDAKELAAALYEHLHVDEKRIDGQPKRNPKTDKFAPGLVVARALGIESSVLPRGMARLARNWGEEEIQTYFVVDVAASVKEVAKAAVSAAQAFDPPRQVSGRSLSPKVGFALAEHLERVTGSKRCSFDPAAGPSVLALHDEVKKTYKRLCARGKNAARAFPADKTELLALMRHTHENRVRNQMVRMGRVSEYRGQQAGDLAQSHYWTSAGQTEIKESEIFVRLWVGAFALAGRSMKAWIDPMGKIVNTEKNDRDLTAAVNIRQVISNKEMVAEAMARRGIYFGETPELDRLGAEGNEGFVFALLRYLRGCRNQTFHLGARAGFLKEIRKELEKTRWGKAKEAEHVVLTDKTVAAIRAIIDNDAKALGARLLADLSGAFVAHYASKEHFSTLYSEIVKAVKDAPEVSSGLPRLKLLLKRADGVRGYVHGLRDTRKHAFATKLPPPPAPRELDDPATKARYIALLRLYDGPFRAYASGITGTALAGPAARAKEAATALAQSVNVTKAYSDVMEGRSSRLRPPNDGETLREYLSALTGETATEFRVQIGYESDSENARKQAEFIENYRRDMLAFMFEDYIRAKGFDWILKIEPGATAMTRAPVLPEPIDTRGQYEHWQAALYLVMHFVPASDVSNLLHQLRKWEALQGKYELVQDGDATDQADARREALDLVKRFRDVLVLFLKTGEARFEGRAAPFDLKPFRALFANPATFDRLFMATPTTARPAEDDPEGDGASEPELRVARTLRGLRQIARYNHMAVLSDLFAKHKVRDEEVARLAEIEDETQEKSQIVAAQELRTDLHDKVMKCHPKTISPEERQSYAAAIKTIEEHRFLVGRVYLGDHLRLHRLMMDVIGRLIDYAGAYERDTGTFLINASKQLGAGADWAVTIAGAANTGARTQTRKDLAHFNVLDRADGTPDLTALVNRAREMMAYDRKRKNAVPRSILDMLARLGLTLKWQMKDHLLQDATITQAAIKHLDKVRLTVGGPAAVTEARFSQDYLQMVAAVFNGSVQNPKPRRRDDGDAWHKPPKPATAQSQPDQKPPNKAPSAGSRLPPPQVGEVYEGVVVKVIDTGSLGFLAVEGVAGNIGLHISRLRRIREDAIIVGRRYRFWVEIYVPPKSNTSKLNAADLVRID

SEQ ID NO: 58 - DNA, 36 bp, synthetic construct; identical to SEQ ID NO: 55
  misc_feature 1..36: note = RcrCas13a direct repeat
  source 1..36: mol_type = other DNA

SEQ ID NO: 59 - DNA, 3858 bp, synthetic construct
  misc_feature 1..3858: note = RcdCas13a nucleic acid sequence
  source 1..3858: mol_type = other DNA
atgcagatcggcaaagtgcagggaaggactattagcgagttcggggacccagcaggaggactgaagaggaagatttctactgacggaaagaaccggaaagaactgccagctcacctgtcaagcgatccaaaggctctgattggccagtggattagtggcattgacaagatctaccggaaaccagatagcagaaaatcagacggcaaggctatccacagtcctacaccaagcaaaatgcagttcgacgcccgggatgatctgggggaagcattctggaaactggtgagtgaagcagggctggcacaggactcagattatgatcagtttaagcgcaggctgcacccttatggggacaagtttcagcccgctgacagcggcgcaaaactgaagttcgaggccgatccccctgagccacaggctttccacggacgctggtatggggccatgtcaaagagggggaacgacgcaaaggaactggcagctgcactgtatgagcatctgcatgtggacgaaaaaaggattgatggccagccaaagaggaacccaaagactgataaatttgcccccgggctggtggtcgcaagggcactggggatcgagagctcagtgctgccaagggggatggccaggctggccaggaactggggagaggaggaaattcagacttatttcgtcgtggacgtggcagcctcagtgaaggaagtggccaaggctgccgtgtccgctgctcaggcatttgacccacctcgacaggtgtcaggacggagcctgtctccaaaggtcgggtttgccctggcagagcacctggaaagggtcaccggaagcaaaagatgcagctttgatccagcagctgggccaagtgtcctggcactgcacgacgaggtgaaaaagacctataagcgactgtgcgccagagggaaaaatgccgcaagagcttttccagcagacaagacagagctgctggcactgatgagacacacacacgaaaatagggtgcgcaaccagatggtcaggatggggagggtcagcgagtatcggggacagcaggccggggatctggctcagagccattattggactagcgcagggcagaccgagattaaggagagcgaaatcttcgtgcggctgtgggtcggggcatttgccctggcaggccgatccatgaaggcctggatcgaccctatggggaagatcgtgaacactgagaagaacgaccgggacctgactgcagcagtgaacatcaggcaggtcatcagcaataaggaaatggtcgccgaggcaatggctagaaggggaatctacttcggggagactccagagctggaccgactgggggcagaaggaaacgaggggttcgtgttcgctctgctgcgatacctgaggggctgtagaaatcagacatttcatctgggggcaagagctggctttctgaaagagattcggaaagagctggaaaagacccggtgggggaaggccaaagaggcagagcatgtggtcctgacagacaaaactgtggcagctatccgagctatcatcgacaatgatgccaaagcactgggggccagactgctggcagatctgtccggcgcctttgtcgcccactacgcaagcaaggagcacttttctacactgtattctgagatcgtcaaggctgtgaaggatgcacctgaagtcagttcaggactgcccaggctgaagctgctgctgaaaagggccgatggggtgcgcgggtatgtccacgggctgagggatacaagaaaacatgccttcgcaacaaagctgccacccccacctgccccaagagagctggacgatcccgcaaccaaggccaggtacatcgccctgctgcgcctgtatgacggcccattcagagcttacgcatccgggattacagggactgcactggctggcccagcagctagggccaaagaggcagcaacagctctggctcagtccgtgaatgtcactaaggcctactctgacgtgatggaaggacggtcatcacggctgaggcctcccaatgacggagagactctgagagaatacctgtctgcactgactggggaaactgcaacagagtttcgggtccagatcggatacgaaagcgacagcgaaaacgcaagaaaacaggctgagtttattgagaattaccggagagatatgctggcttttatgttcgaggattatatcagagccaaggggttcgattggattctgaaaattgagcccggggcaacagccatgactagggcaccagtgctgcccgaacctatcgatacacgagggcagtacgagcattggcaggctgcactgtatctggtcatgcatttcgtgccagcctcagacgtgtctaacctgctgcaccagctgcggaaatgggaagccctgcaggggaagtacgagctggtccaggacggggacgccacagaccaggccgacgcccggcgggaagctctggacctggtcaagcggttccgagatgtcctggtgctgttcctgaaaactggcgaggcacgcttcgagggaagggctgctccttttgacctgaagccattccgagctctgtttgccaaccccgcaacattcgatcgcctgtttatggcaacacctaccacagctcgaccagccgaggatgatccagagggggacggggcatccgagcctgaactgcgggtcgcaaggacactgcgggggctgcggcagatcgcacggtacaatcacatggcagtcctgtcagacctgttcgccaaacataaggtgcgcgatgaagaggtggcaaggctggccgagattgaggatgaaacccaggagaagtcacagatcgtcgccgcacaggagctgcggaccgacctgcacgacaaggtcatgaagtgccaccctaagacaattagccccgaggagcgccagagctacgcagcagccatcaaaactatcgaagagcatagatttctggtggggagggtctatctgggggatcatctgcgactgcaccggctgatgatggacgtgatcggccgcctgattgactatgctggagcctatgaaagagatactggcacattcctgatcaatgcaagcaaacagctgggggccggcgcagactgggcagtgacaatcgccggggccgccaatacagatgctagaactcagacacggaaggatctggctcacttcaacgtgctggatcgggctgatgggacccccgacctgactgcactggtgaatcgcgcacgggaaatgatggcctacgatcggaagagaaaaaatgccgtcccaaggagcattctggatatgctggcacggctgggactgaccctgaagtggcagatgaaggatcacctgctgcaggatgcaaccattacccaggcagctatcaagcacctggacaaggtgagactgaccgtgggcggaccagcagcagtcacagaagctcggttctctcaggactacctgcagatggtggctgcagtgtttaacggatcagtgcagaaccccaagccacggcgcagggacgacggagatgcttggcataaacctcctaagccagccaccgcccagagccagccagatcagaaacctcccaacaaagcaccttccgcagggtcccggctgccccctccacaggtgggagaggtctacgaaggcgtggtcgtcaaagtcattgatactggatctctggggttcctggctgtcgaaggcgtcgctggaaatattggcctgcacatctcccgactgaggcggattcgggaggatgctatcattgtcggacgaaggtataggttccgggtggaaatctacgtgcctccaaagagcaacacttccaaactgaacgctgccgacctggtgcggattgattga

SEQ ID NO: 60 - protein, 1285 aa, synthetic construct
  REGION 1..1285: note = RcdCas13a amino acid sequence
MQIGKVQGRTISEFGDPAGGLKRKISTDGKNRKELPAHLSSDPKALIGQWISGIDKIYRKPDSRKSDGKAIHSPTPSKMQFDARDDLGEAFWKLVSEAGLAQDSDYDQFKRRLHPYGDKFQPADSGAKLKFEADPPEPQAFHGRWYGAMSKRGNDAKELAAALYEHLHVDEKRIDGQPKRNPKTDKFAPGLVVARALGIESSVLPRGMARLARNWGEEEIQTYFVVDVAASVKEVAKAAVSAAQAFDPPRQVSGRSLSPKVGFALAEHLERVTGSKRCSFDPAAGPSVLALHDEVKKTYKRLCARGKNAARAFPADKTELLALMRHTHENRVRNQMVRMGRVSEYRGQQAGDLAQSHYWTSAGQTEIKESEIFVRLWVGAFALAGRSMKAWIDPMGKIVNTEKNDRDLTAAVNIRQVISNKEMVAEAMARRGIYFGETPELDRLGAEGNEGFVFALLRYLRGCRNQTFHLGARAGFLKEIRKELEKTRWGKAKEAEHVVLTDKTVAAIRAIIDNDAKALGARLLADLSGAFVAHYASKEHFSTLYSEIVKAVKDAPEVSSGLPRLKLLLKRADGVRGYVHGLRDTRKHAFATKLPPPPAPRELDDPATKARYIALLRLYDGPFRAYASGITGTALAGPAARAKEAATALAQSVNVTKAYSDVMEGRSSRLRPPNDGETLREYLSALTGETATEFRVQIGYESDSENARKQAEFIENYRRDMLAFMFEDYIRAKGFDWILKIEPGATAMTRAPVLPEPIDTRGQYEHWQAALYLVMHFVPASDVSNLLHQLRKWEALQGKYELVQDGDATDQADARREALDLVKRFRDVLVLFLKTGEARFEGRAAPFDLKPFRALFANPATFDRLFMATPTTARPAEDDPEGDGASEPELRVARTLRGLRQIARYNHMAVLSDLFAKHKVRDEEVARLAEIEDETQEKSQIVAAQELRTDLHDKVMKCHPKTISPEERQSYAAAIKTIEEHRFLVGRVYLGDHLRLHRLMMDVIGRLIDYAGAYERDTGTFLINASKQLGAGADWAVTIAGAANTDARTQTRKDLAHFNVLDRADGTPDLTALVNRAREMMAYDRKRKNAVPRSILDMLARLGLTLKWQMKDHLLQDATITQAAIKHLDKVRLTVGGPAAVTEARFSQDYLQMVAAVFNGSVQNPKPRRRDDGDAWHKPPKPATAQSQPDQKPPNKAPSAGSRLPPPQVGEVYEGVVVKVIDTGSLGFLAVEGVAGNIGLHISRLRRIREDAIIVGRRYRFRVEIYVPPKSNTSKLNAADLVRID

SEQ ID NO: 61 - DNA, 36 bp, synthetic construct; identical to SEQ ID NO: 55
  misc_feature 1..36: note = RcdCas13a direct repeat
  source 1..36: mol_type = other DNA

SEQ ID NO: 62 - DNA, 3537 bp, synthetic construct
  misc_feature 1..3537: note = PguCas13b nucleic acid sequence
  source 1..3537: mol_type = other DNA
atgacagagcagagcgagaggccctacaacggcacctactacaccctggaagataagcacttctgggccgcctttctgaacctggccagacacaacgcctacatcaccctgacacacatcgaccggcagctggcctacagcaaggccgacatcaccaacgaccaggacgtgctgagcttcaaggccctgtggaagaacttcgacaacgacctggaaagaaagagccggctgcggagcctgatcctgaagcacttcagctttctggaaggcgccgcttacggcaagaagctgttcgagtctaagagcagcggcaacaagagcagcaagaacaaagagctgaccaagaaagagaaagaggaactgcaggccaacgctctgagcctggacaacctgaagtctatcctgttcgacttcctgcagaagctgaaggacttccggaactactacagccactaccggcacagcggaagcagcgagctgcctctgttcgacggcaacatgctgcagcggctgtacaacgtgttcgacgtgtccgtgcagcgcgtgaagatcgaccacgagcacaacgatgaggtggaccctcactaccacttcaaccacctcgtgcggaagggcaagaaggacagatacggccacaacgacaaccccagcttcaagcaccacttcgtggacggcgaaggcatggttacagaagccggcctgctgttcttcgtgtccctgttcctggaaaagcgggacgccatctggatgcagaagaagatccggggcttcaaaggcggcaccgagacataccagcagatgaccaacgaggtgttctgccggtccagaatcagcctgcctaagctgaagctggaaagcctgcggatggacgactggatgctgctggacatgctgaacgaactcgtgcggtgccccaagcctctgtacgacagactgagagaggacgaccgggcctgctttagagtgcccgtggatatcctgcctgacgaggacgatacagatggcggcggagaggaccccttcaagaataccctcgtccggcaccaggacagattcccttacttcgccctgcggtacttcgacctgaagaaggtgttcaccagcctgcggttccacatcgatctgggcacctaccactttgccatctacaagaagatgatcggcgagcagcccgaggacagacacctgaccagaaacctgtacggcttcggccggatccaggacttcgccgaagaacacagacccgaggaatggaagcggctcgtcagagatctggactacttcgagacaggcgacaagccctacatcagccagacaagcccacactaccacatcgagaagggaaagatcggcctgagattcatgcccgagggccagcatctttggccctctccagaagtgggcaccaccagaaccggcagatctaagtacgcccaggacaagagactgaccgccgaggcctttctgtctgtgcacgagctgatgcctatgatgttctactacttcctgctgcgcgagaagtacagcgaggaagtgtctgccgagagagtgcagggcagaatcaagcgcgtgatcgaggatgtgtacgccgtgtacgatgcctttgccagggacgagatcaacacccgggatgagctggatgcctgcctggccgataagggcatcagaagaggccatctgcctcgccagatgatcgccatcctgagccaagagcacaaggacatggaagagaagattcggaagaaactgcaagagatgatggccgacaccgaccaccggctggatatgctggatagacagaccgaccggaagatcagaatcggccggaagaatgccggactgcctaagagcggagtgatcgccgattggctcgtgcgggacatgatgagatttcagcccgtggccaaggacgccagcggaaagcctctgaacaactccaaggccaacagcaccgagtaccggatgctgcagagagccctggctctgtttggaggcgagaaagagaggctgaccccatacttccggcagatgaatctgaccggcggaaacaaccctcatccttttctgcacgaaactcgctgggagagccacaccaacatcctgtccttttaccggtcctacctgagagcccgcaaggccttcctcgaaaggatcggcagatccgacagagtggaaaaccggccatttctgctgctgaaagagcccaagaccgacagacagacactggtggctggatggaagggcgagttccatctgccaagaggcatcttcacagaggccgtgcgggattgcctgatcgagatgggacacgatgaagtggccagctacaaagaagtgggattcatggccaaggccgtgccactgtactttgagagagcctgcgaggacagggtgcagcccttttacgacagccccttcaacgtgggcaacagcctgaagcctaagaagggcagattcctgagcaaagaggaacgggccgaagagtgggagcggggcaaagagagattccgggatctcgaagcctggtcctatagcgccgccagaagaatcgaggacgcctttgccggcatcgagtatgctagccccggaaacaagaagaaaatcgagcagctcctgcgggacctgagcctgtgggaagcctttgagagcaagctgaaagtgcgggccgaccggatcaatctggccaagctgaagaaagaaatcctggaagcccaagaacacccctaccacgacttcaagagctggcagaagttcgagcgcgagctgcggctggtcaagaaccaggatatcatcacctggatgatgtgccgcgacctgatggaagaaaacaaggtggaaggcctggacaccggcacactgtacctgaaggacatcagacccaacgtgcaagagcagggcagcctgaacgtgctgaacagagtgaagcccatgagactgcccgtggtggtgtacagagccgatagcagaggccacgtgcacaaagaagaagcccctctggccaccgtgtacatcgaggaaagagacaccaagctgctgaagcagggcaacttcaagtccttcgtgaaggacaggcggctgaacggcctgttctccttcgttgatactggcggactggccatggaacagtaccccatctccaagctgagagtggaatacgagctggccaagtaccagaccgccagagtgtgtgtgttcgagctgaccctgagactggaagagtccctgctgacccggtatcctcatctgcccgacgagagcttcagagagatgctggaatcttggagcgaccctctgctggccaaatggcctgaactgcacggaaaagtgcggctgctgatcgccgtgcggaatgcctttagccacaatcagtaccctatgtacgacgaggccgtgttcagctccatccggaagtacgatcctagcagccccgacgccatcgaagagagaatgggcctgaatatcgcccaccgcctgtctgaggaagtgaagcaggccaaagaaaccgtggaacggatcattcaggccggatcccttcaa

SEQ ID NO: 63 - protein, 1179 aa, synthetic construct
  REGION 1..1179: note = PguCas13b amino acid sequence
MTEQSERPYNGTYYTLEDKHFWAAFLNLARHNAYITLTHIDRQLAYSKADITNDQDVLSFKALWKNFDNDLERKSRLRSLILKHFSFLEGAAYGKKLFESKSSGNKSSKNKELTKKEKEELQANALSLDNLKSILFDFLQKLKDFRNYYSHYRHSGSSELPLFDGNMLQRLYNVFDVSVQRVKIDHEHNDEVDPHYHFNHLVRKGKKDRYGHNDNPSFKHHFVDGEGMVTEAGLLFFVSLFLEKRDAIWMQKKIRGFKGGTETYQQMTNEVFCRSRISLPKLKLESLRMDDWMLLDMLNELVRCPKPLYDRLREDDRACFRVPVDILPDEDDTDGGGEDPFKNTLVRHQDRFPYFALRYFDLKKVFTSLRFHIDLGTYHFAIYKKMIGEQPEDRHLTRNLYGFGRIQDFAEEHRPEEWKRLVRDLDYFETGDKPYISQTSPHYHIEKGKIGLRFMPEGQHLWPSPEVGTTRTGRSKYAQDKRLTAEAFLSVHELMPMMFYYFLLREKYSEEVSAERVQGRIKRVIEDVYAVYDAFARDEINTRDELDACLADKGIRRGHLPRQMIAILSQEHKDMEEKIRKKLQEMMADTDHRLDMLDRQTDRKIRIGRKNAGLPKSGVIADWLVRDMMRFQPVAKDASGKPLNNSKANSTEYRMLQRALALFGGEKERLTPYFRQMNLTGGNNPHPFLHETRWESHTNILSFYRSYLRARKAFLERIGRSDRVENRPFLLLKEPKTDRQTLVAGWKGEFHLPRGIFTEAVRDCLIEMGHDEVASYKEVGFMAKAVPLYFERACEDRVQPFYDSPFNVGNSLKPKKGRFLSKEERAEEWERGKERFRDLEAWSYSAARRIEDAFAGIEYASPGNKKKIEQLLRDLSLWEAFESKLKVRADRINLAKLKKEILEAQEHPYHDFKSWQKFERELRLVKNQDIITWMMCRDLMEENKVEGLDTGTLYLKDIRPNVQEQGSLNVLNRVKPMRLPVVVYRADSRGHVHKEEAPLATVYIEERDTKLLKQGNFKSFVKDRRLNGLFSFVDTGGLAMEQYPISKLRVEYELAKYQTARVCVFELTLRLEESLLTRYPHLPDESFREMLESWSDPLLAKWPELHGKVRLLIAVRNAFSHNQYPMYDEAVFSSIRKYDPSSPDAIEERMGLNIAHRLSEEVKQAKETVERIIQAGSLQ

SEQ ID NO: 64 - DNA, 36 bp, synthetic construct
  misc_feature 1..36: note = PguCas13b direct repeat
  source 1..36: mol_type = other DNA
gttggatctaccctctatttgaagggtacacacaac

SEQ ID NO: 65 - DNA, 2943 bp, synthetic construct
  misc_feature 1..2943: note = RfxCas13d-SV40NLS nucleic acid sequence
  source 1..2943: mol_type = other DNA
atgagccccaagaagaagagaaaggtggaggccagcatcgaaaaaaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactggcgcactgggtggtcgctaacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcgcaaacaaggccgtcgccctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaattccggatccgga

SEQ ID NO: 66 - protein, 981 aa, synthetic construct
  REGION 1..981: note = RfxCas13d-SV40NLS amino acid sequence
MSPKKKRKVEASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSGSG

SEQ ID NO: 67 - DNA, 30 bp, synthetic construct
  misc_feature 1..30: note = RfxCas13d direct repeat
  source 1..30: mol_type = other DNA
aacccctaccaactggtcggggtttgaaac

SEQ ID NO: 68 - DNA, 24 bp, synthetic construct
  misc_feature 1..24: note = SV40 Nuclear Localization Signaling nucleic acid sequence
  source 1..24: mol_type = other DNA
ccaaagaagaagcggaaggtcggt

SEQ ID NO: 69 - protein, 7 aa, synthetic construct
  REGION 1..7: note = SV40 Nuclear Localization Signaling amino acid sequence
PKKKRKV

SEQ ID NO: 70 - DNA, 1479 bp, synthetic construct
  misc_feature 1..1479: note = CMV-BD-2xms2-PPT-exon-bGHpA repair template
  source 1..1479: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcggatcttccatttcgggtgtcgtgacgtacgaatacgatctagaccctgcatcctctctgggacactcaacaccctcacggacagcccgaattcgacccaagctgacgggagcacatgaggatcacccatgtgccacgagcgacatgaggatcacccatgtcgctttcactagtctgtggtgtgatatccatggcggcctacttatcctgtcccttttttttccacaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 71 - DNA, 3357 bp, synthetic construct
  misc_feature 1..3357: note = PinCas13b nucleic acid sequence
  source 1..3357: mol_type = other DNA
atgaccgagcagaacgagaagccctacaacggcacctactacaccctggaagataagcacttctgggccgcctttctgaacctggccagacacaacgcctacatcacactggcccacatcgacagacagctggcctacagcaaggccgacatcaccaacgacgaggacatcctgttcttcaaaggccagtggaagaacctggacaacgacctggaacggaaggccagactgagaagcctgatcctgaagcacttcagctttctggaaggcgccgcttacggcaagaagctgtttgagtctcagagcagcggcaacaagagcagcaagaagaaagagctgtccaagaaagagaaagaggaactgcaggccaacgctctgagcctggacaatctgaagtctatcctgttcgacttcctgcagaagctgaaggacttccggaactactacagccactacagacaccccgagagcagcgagctgcctctgttcgatggcaacatgctgcagcggctgtacaacgtgttcgacgtgtccgtgcagcgcgtgaagagagatcacgagcacaacgacaaggtggaccctcaccggcacttcaatcacctcgtgcggaagggcaagaaggataagtacggcaacaacgataacccgttcttcaagcaccacttcgtggaccgcgagggcacagtgacagaagccggactgctgttcttcgtgtccctgttcctggaaaagcgggacgccatctggatgcagaagaagatccggggctttaaaggcggcaccgaggcctaccagcagatgaccaatgaggtgttctgccgcagccggatcagcctgcctaagctgaagctggaaagcctgagaaccgacgactggatgctgctggacatgctgaacgaactcgtgcggtgccacaagagcctgtacgatagactgagagaagaggaccgggccagattcagagtgcccgtggatatcctgagcgacgaggatgataccgacggcaccgaagaggatcccttcaagaacactctcgtgcggcaccaggacagattcccttacttcgccctgcggtacttcgacctgaagaaggtgttcaccagcctgcggttccacatcgatctgggcacctaccacttcgccatctacaagaagaacatcggcgagcagcccgaggacagacacctgaccagaaacctgtacggcttcggccggatccaggacttcgccgaagaacacagacccgaggaatggaagcggctcgtcagagatctggactacttcgagacaggcgacaagccttacatcacccagaccacacctcactaccacatcgagaagggaaagatcggcctgagattcgtgcccgagggacagcatctttggccctctccagaagtgggcgccaccagaacaggcagatctaagtacgcccaggacaagagactgaccgccgaggcctttctgtctgtgcacgagctgatgcctatgatgttctactacttcctgctgcgcgagaagtacagcgaggaagtgtctgccgagaaggtgcagggcagaatcaagcgcgtgatcgaggatgtgtacgccgtgtacgatgccttcgccagggacgagatcaacaccagagatgagctggatgcctgcctggccgacaagggcattagaagaggccatctgccaagacagatgatcgccatcctgagccaagagcacaaggacatggaagagaaagtgcggaagaaactgcaagagatgattgccgacaccgaccaccggctggatatgctggatagacagaccgaccggaagatcagaatcggccggaaaaatgccggcctgccaaagtctggcgtggtggctgattggctcgtgcgggacatgatgagattccagcctgtggccaaggacaccagcggcaagcctctgaacaactccaaggccaacagcaccgagtaccggatgctgcagagagccctggctctgtttggaggcgagaaagagaggctgaccccatacttccggcagatgaatctgaccggcggaaacaaccctcatccttttctgcacgaaactcgctgggagagccacaccaatatcctgtccttctaccggtcctacctggaagcccggaaggctttcctgcagtccatcggcagatccgacagagtggaaaaccaccggtttctgctgctgaaagagcccaagaccgacaggcagactctggtggctggatggaagggcgagttccatctgcctagaggcatctttacagaggccgtgcgcgactgcctgatcgagatgggctatgatgaagtgggcagctacaaagaagtgggattcatggccaaggccgtgcctctgtactttgagagagccagcaaggaccgggtgcagcccttctacgactaccccttcaacgtgggcaacagcctgaagcctaagaagggcagattcctgtccaaagagaagcgggccgaagagtgggagagcggcaaagagagattccggctggccaagctgaagaaagaaatcctcgaagccaaagagcacccctaccacgacttcaagagctggcagaagttcgagcgcgagctgagactggtcaagaaccaggacatcatcacctggatgatgtgccgggacctgatggaagaaaacaaagtggaaggcctggatacaggcaccctgtacctgaaggacatcaggaccgacgtgcaagagcagggcagcctgaacgtgctgaacagagtgaagcccatgagactgcccgtggtggtgtacagagccgatagcagaggccacgtgcacaaagaacaggcccctctggccaccgtgtacatcgaggaaagagacaccaagctgctgaagcagggcaacttcaagtccttcgtgaaggacagacggctgaatggcctgttcagcttcgtggatactggcgccctggccatggaacagtaccccatctctaaactgcgcgtggaatacgagctggccaaataccagaccgccagagtgtgcgccttcgagcagacactggaactggaagagagcctgctgacccggtatcctcatctgcccgacaagaactttcggaagatgctcgaatcttggagcgaccctctgctggataagtggcctgacctgcacggaaatgtgcggctgctgatcgccgtgcggaatgcctttagccacaatcagtaccctatgtacgacgagacactgtttagctccatccggaagtacgaccctagcagccctgacgccattgaggaacggatgggcctgaatatcgcccacagactgtctgaggaagtgaagcaggccaaagaaatggtggaacggatcattcaggcc

SEQ ID NO: 72 - protein, 1119 aa, synthetic construct
  REGION 1..1119: note = PinCas13b amino acid sequence
MTEQNEKPYNGTYYTLEDKHFWAAFLNLARHNAYITLAHIDRQLAYSKADITNDEDILFFKGQWKNLDNDLERKARLRSLILKHFSFLEGAAYGKKLFESQSSGNKSSKKKELSKKEKEELQANALSLDNLKSILFDFLQKLKDFRNYYSHYRHPESSELPLFDGNMLQRLYNVFDVSVQRVKRDHEHNDKVDPHRHFNHLVRKGKKDKYGNNDNPFFKHHFVDREGTVTEAGLLFFVSLFLEKRDAIWMQKKIRGFKGGTEAYQQMTNEVFCRSRISLPKLKLESLRTDDWMLLDMLNELVRCHKSLYDRLREEDRARFRVPVDILSDEDDTDGTEEDPFKNTLVRHQDRFPYFALRYFDLKKVFTSLRFHIDLGTYHFAIYKKNIGEQPEDRHLTRNLYGFGRIQDFAEEHRPEEWKRLVRDLDYFETGDKPYITQTTPHYHIEKGKIGLRFVPEGQHLWPSPEVGATRTGRSKYAQDKRLTAEAFLSVHELMPMMFYYFLLREKYSEEVSAEKVQGRIKRVIEDVYAVYDAFARDEINTRDELDACLADKGIRRGHLPRQMIAILSQEHKDMEEKVRKKLQEMIADTDHRLDMLDRQTDRKIRIGRKNAGLPKSGVVADWLVRDMMRFQPVAKDTSGKPLNNSKANSTEYRMLQRALALFGGEKERLTPYFRQMNLTGGNNPHPFLHETRWESHTNILSFYRSYLEARKAFLQSIGRSDRVENHRFLLLKEPKTDRQTLVAGWKGEFHLPRGIFTEAVRDCLIEMGYDEVGSYKEVGFMAKAVPLYFERASKDRVQPFYDYPFNVGNSLKPKKGRFLSKEKRAEEWESGKERFRLAKLKKEILEAKEHPYHDFKSWQKFERELRLVKNQDIITWMMCRDLMEENKVEGLDTGTLYLKDIRTDVQEQGSLNVLNRVKPMRLPVVVYRADSRGHVHKEQAPLATVYIEERDTKLLKQGNFKSFVKDRRLNGLFSFVDTGALAMEQYPISKLRVEYELAKYQTARVCAFEQTLELEESLLTRYPHLPDKNFRKMLESWSDPLLDKWPDLHGNVRLLIAVRNAFSHNQYPMYDETLFSSIRKYDPSSPDAIEERMGLNIAHRLSEEVKQAKEMVERIIQA

SEQ ID NO: 73 - DNA, 36 bp, synthetic construct
  misc_feature 1..36: note = PinCas13b direct repeat
  source 1..36: mol_type = other DNA
gttgcatctgcctgctgtttgcaaggtaaaaacaac

SEQ ID NO: 74 - DNA, 3579 bp, synthetic construct
  misc_feature 1..3579: note = BzoCas13b nucleic acid sequence
  source 1..3579: mol_type = other DNA
atggagaataccgactccgtgttccgggagctgggcaagagactgaagggcaaggagtacacatctgagaacttctttgacgccatctttaaggagaatatctctctggtggagtacgagcggtatgtgaagctgctgagcgattatttccccatggcccggctgctggacaagaaggaggtgcctatcaaggagagaaaggagaacttcaagaagaacttcaagggcatcatcaaggccgtgcgggatctgagaaacttttacacccacaaggagcacggcgaggtggagatcacagacgagatcttcggcgtgctggatgagatgctgaagtccaccgtgctgacagtgaagaagaagaaggtgaaaaccgacaagacaaaggagatcctgaagaagtctatcgagaagcagctggatatcctgtgccagaagaagctggagtacctgagggacaccgcccgcaagatcgaggagaagcggagaaaccagagggagaggggagagaaggagctggtggcaccttttaagtattctgataagcgggacgacctgatcgccgccatctacaatgacgccttcgacgtgtatatcgacaagaagaaggattccctgaaggagagctccaaggccaagtacaacacaaagtctgaccctcagcaggaggagggcgatctgaagatcccaatcagcaagaatggcgtggtgttcctgctgtccctgtttctgaccaagcaggagatccacgccttcaagagcaagatcgccggctttaaggccacagtgatcgacgaggccaccgtgtccgaggccacagtgtctcacggcaagaacagcatctgtttcatggccacccacgagatctttagccacctggcctacaagaagctgaagaggaaggtgcgcacagccgagatcaactacggcgaggccgagaatgccgagcagctgtccgtgtatgccaaggagacactgatgatgcagatgctggacgagctgtctaaggtgcctgatgtggtgtaccagaacctgagcgaggacgtgcagaaaaccttcatcgaggactggaatgagtatctgaaggagaacaatggcgacgtgggcacaatggaggaggagcaggtcatccacccagtgatccggaagagatacgaggataagttcaactattttgccatcaggttcctggacgagttcgcccagtttcctaccctgcgctttcaggtgcacctgggcaactacctgcacgattcccggccaaaggagaatctgatctctgacaggcgcatcaaggagaagatcacagtgtttggcagactgtccgagctggagcacaagaaggccctgttcatcaagaacaccgagacaaatgaggatagggagcactactgggagatcttccccaaccctaattatgactttccaaaggagaacatctccgtgaatgacaaggattttcccatcgccggctctatcctggatcgcgagaagcagcctgtggccggcaagatcggcatcaaggtgaagctgctgaaccagcagtacgtgtctgaggtggacaaggccgtgaaggcccaccagctgaagcagcggaaggcctctaagcccagcatccagaatatcatcgaggagatcgtgcccatcaacgagagcaatcctaaggaggcaatcgtgttcggaggacagcctaccgcctacctgtctatgaacgatatccacagcatcctgtatgagttctttgacaagtgggagaagaagaaggagaagctggagaagaagggcgagaaggagctgagaaaggagatcggcaaggagctggagaagaagatcgtgggcaagatccaggcccagatccagcagatcatcgacaaggataccaacgccaagatcctgaagccctaccaggacggcaatagcacagccatcgataaggagaagctgatcaaggacctgaagcaggagcagaacatcctgcagaagctgaaggatgagcagaccgtgagggagaaggagtacaatgacttcatcgcctatcaggataagaaccgcgagatcaataaggtgagggatcgcaaccacaagcagtacctgaaggacaatctgaagaggaagtatcctgaggccccagccaggaaggaggtgctgtactatcgcgagaagggcaaggtggccgtgtggctggccaacgatatcaagcggttcatgcctaccgactttaagaatgagtggaagggcgagcagcactccctactgcagaagtctctggcctactatgagcagtgcaaggaggagctgaagaacctgctgccagagaaggtgttccagcacctgccctttaagctgggcggctacttccagcagaagtacctgtatcagttttacacctgctatctggacaagaggctggagtatatcagcggcctggtgcagcaggccgagaacttcaagtccgagaataaggtgtttaagaaggtggagaacgagtgtttcaagtttctgaagaagcagaattacacacacaaggagctggatgcccgggtgcagtccatcctgggctatccaatcttcctggagagaggctttatggacgagaagcccaccatcatcaagggcaagacattcaagggcaacgaggccctgttcgccgattggtttcgctactataaggagtaccagaacttccagaccttttacgacacagagaattatccactggtggagctggagaagaagcaggccgatcggaagagaaagaccaagatctatcagcagaagaagaatgacgtgtttacactgctgatggccaagcacatcttcaagagcgtgttcaagcaggacagcatcgatcagttctccctggaggacctgtaccagagccgggaggagagactgggaaaccaggagagggcaaggcagaccggcgagaggaacacaaattatatctggaataagaccgtggacctgaagctgtgcgatggcaagatcacagtggagaacgtgaagctgaagaatgtgggcgatttcatcaagtacgagtatgaccagcgggtgcaggcctttctgaagtacgaggagaacatcgagtggcaggccttcctgatcaaggagagcaaggaggaggagaattacccctatgtggtggagagagagatcgagcagtacgagaaggtgcggagagaggagctgctgaaggaggtgcacctgatcgaggagtatatcctggagaaggtgaaggataaagaaatcctgaagaagggcgacaaccagaacttcaagtactatatcctgaacggcctgctgaagcagctgaagaatgaggatgtggagtcctacaaggtgttcaacctgaataccgagccagaggatgtgaacatcaatcagctgaagcaggaggccaccgacctggagcagaaggccttcgtgctgacatatatccggaacaagtttgcccacaatcagctgcccaagaaggagttctgggactactgtcaggagaagtatggcaagatcgagaaggagaaaacctacgccgagtatttcgccgaggtgtttaagaaggagaaggaggccctgatcaag

SEQ ID NO: 75 - protein, 1193 aa, synthetic construct
  REGION 1..1193: note = BzoCas13b amino acid sequence
MENTDSVFRELGKRLKGKEYTSENFFDAIFKENISLVEYERYVKLLSDYFPMARLLDKKEVPIKERKENFKKNFKGIIKAVRDLRNFYTHKEHGEVEITDEIFGVLDEMLKSTVLTVKKKKVKTDKTKEILKKSIEKQLDILCQKKLEYLRDTARKIEEKRRNQRERGEKELVAPFKYSDKRDDLIAAIYNDAFDVYIDKKKDSLKESSKAKYNTKSDPQQEEGDLKIPISKNGVVFLLSLFLTKQEIHAFKSKIAGFKATVIDEATVSEATVSHGKNSICFMATHEIFSHLAYKKLKRKVRTAEINYGEAENAEQLSVYAKETLMMQMLDELSKVPDVVYQNLSEDVQKTFIEDWNEYLKENNGDVGTMEEEQVIHPVIRKRYEDKFNYFAIRFLDEFAQFPTLRFQVHLGNYLHDSRPKENLISDRRIKEKITVFGRLSELEHKKALFIKNTETNEDREHYWEIFPNPNYDFPKENISVNDKDFPIAGSILDREKQPVAGKIGIKVKLLNQQYVSEVDKAVKAHQLKQRKASKPSIQNIIEEIVPINESNPKEAIVFGGQPTAYLSMNDIHSILYEFFDKWEKKKEKLEKKGEKELRKEIGKELEKKIVGKIQAQIQQIIDKDTNAKILKPYQDGNSTAIDKEKLIKDLKQEQNILQKLKDEQTVREKEYNDFIAYQDKNREINKVRDRNHKQYLKDNLKRKYPEAPARKEVLYYREKGKVAVWLANDIKRFMPTDFKNEWKGEQHSLLQKSLAYYEQCKEELKNLLPEKVFQHLPFKLGGYFQQKYLYQFYTCYLDKRLEYISGLVQQAENFKSENKVFKKVENECFKFLKKQNYTHKELDARVQSILGYPIFLERGFMDEKPTIIKGKTFKGNEALFADWFRYYKEYQNFQTFYDTENYPLVELEKKQADRKRKTKIYQQKKNDVFTLLMAKHIFKSVFKQDSIDQFSLEDLYQSREERLGNQERARQTGERNTNYIWNKTVDLKLCDGKITVENVKLKNVGDFIKYEYDQRVQAFLKYEENIEWQAFLIKESKEEENYPYVVEREIEQYEKVRREELLKEVHLIEEYILEKVKDKEILKKGDNQNFKYYILNGLLKQLKNEDVESYKVFNLNTEPEDVNINQLKQEATDLEQKAFVLTYIRNKFAHNQLPKKEFWDYCQEKYGKIEKEKTYAEYFAEVFKKEKEALIK

SEQ ID NO: 76 - DNA, 36 bp, synthetic construct
  misc_feature 1..36: note = BzoCas13b direct repeat
  source 1..36: mol_type = other DNA
gttggaactgctctcattttggagggtaatcacaac

SEQ ID NO: 77 - DNA, 3381 bp, synthetic construct
  misc_feature 1..3381: note = PbuCas13b nucleic acid sequence
  source 1..3381: mol_type = other DNA
atgcagaagcaggacaagctgttcgtggataggaagaagaacgccatcttcgcctttcccaagtacatcaccatcatggagaataaggagaagcccgagcctatctactatgagctgacagacaagcacttctgggccgcctttctgaacctggcccgccacaacgtgtataccacaatcaaccacatcaatcggagactggagatcgccgagctgaaggacgatggctatatgatgggcatcaagggcagctggaacgagcaggccaagaagctggacaagaaggtgcggctgagagatctgatcatgaagcacttcccttttctggaggccgccgcctacgagatgaccaatagcaagtccccaaacaataaggagcagagggagaaggagcagtctgaggccctgagcctgaacaatctgaagaacgtgctgttcatctttctggagaagctgcaggtgctgcgcaattactatagccactacaagtattctgaggagagcccaaagcccatcttcgagacatccctgctgaagaacatgtataaggtgtttgacgccaatgtgaggctggtgaagcgcgattatatgcaccacgagaacatcgacatgcagagagatttcacccacctgaatcggaagaagcaagtgggcagaacaaagaacatcatcgactcccctaatttccactaccactttgccgataaggagggcaacatgaccatcgccggcctgctgttcttcgtgagcctgttcctggacaagaaggatgccatctggatgcagaagaagctgaagggcttcaaggatggcaggaacctgcgcgagcagatgaccaatgaggtgttttgcaggtcccgcatctctctgccaaagctgaagctggagaacgtgcagacaaaggactggatgcagctggatatgctgaatgagctggtgaggtgtcccaagagcctgtacgagcggctgagagagaaggaccgcgagtctttcaaggtgcctttcgatatctttagcgacgattataacgccgaggaggagccctttaagaataccctggtgcggcaccaggacagattcccttactttgtgctgaggtatttcgatctgaacgagatcttcgagcagctgcgctttcagatcgatctgggcacctaccacttttccatctataataagaggatcggcgacgaggatgaggtgcgccacctgacacaccacctgtacggcttcgccagaatccaggactttgccccccagaaccagtctgaggagtggagaaagctggtgaaggacctggatcacttcgagacaagccaggagccctacatcagcaagacagcccctcactatcacctggagaatgagaagatcggcatcaagttctgcagcgcccacaacaatctgtttccctccctgcagaccgacaagacatgtaacggccggtccaagttcaatctgggcacccagttcacagccgaggcctttctgtctgtgcacgagctgctgcctatgatgttttactatctgctgctgaccaaggactactcccggaaggagtctgccgataaggtggagggcatcatcagaaaggagatctctaacatctacgccatctatgacgccttcgccaacaatgagatcaatagcatcgccgatctgaccaggcgcctgcagaacacaaatatcctgcagggccacctgccaaagcagatgatcagcatcctgaagggcaggcagaaggatatgggcaaggaggccgagcgcaagatcggcgagatgatcgacgatacccagcggagactggacctgctgtgcaagcagacaaaccagaagatcaggatcggcaagcgcaatgccggcctgctgaagtccggcaagatcgccgactggctggtgaacgatatgatgcggttccagcctgtgcagaaggatcagaacaatatcccaatcaacaatagcaaggccaactccaccgagtaccggatgctgcagagagccctggccctgttcggctccgagaacttccggctgaaggcctacttcaaccagatgaatctggtgggcaacgacaatcctcacccatttctggccgagacacagtgggagcaccagacaaacatcctgtctttctacaggaattatctggaggcccgcaagaagtacctgaagggcctgaagccccagaactggaagcagtatcagcactttctgatcctgaaggtgcagaaaaccaacaggaataccctggtgacaggctggaagaacagcttcaatctgccacggggcatctttacacagcccatcagagagtggttcgagaagcacaacaattccaagcggatctacgaccagatcctgtctttcgatagagtgggctttgtggccaaggccatccctctgtactttgccgaggagtataaggacaacgtgcagcctttctacgattatcccttcaacatcggcaatcggctgaagcctaagaagagacagttcctggataagaaggagcgcgtggagctgtggcagaagaacaaggagctgtttaagaattacccatctgagaagaagaaaaccgacctggcctatctggatttcctgagctggaagaagtttgagcgggagctgagactgatcaagaaccaggacatcgtgacctggctgatgttcaaggagctgtttaatatggccacagtggagggcctgaagatcggcgagatccacctgcgggacatcgataccaacacagccaatgaggagtccaacaatatcctgaacagaatcatgccaatgaagctgcccgtgaaaacctacgagacagacaacaagggcaatatcctgaaggagcggccactggccaccttctatatcgaggagacagagacaaaggtgctgaagcagggcaactttaaggccctggtgaaggacaggcgcctgaatggcctgttctcttttgccgagacaacagatctgaatctggaggagcaccccatcagcaagctgtccgtggacctggagctgatcaagtaccagaccacaagaatcagcatcttcgagatgaccctgggcctggagaagaagctgatcgacaagtattccaccctgccaacagattcttttaggaacatgctggagcggtggctgcagtgtaaggccaataggcccgagctgaagaactacgtgaatagcctgatcgccgtgcgcaacgccttctcccacaatcagtaccctatgtatgatgccacactgtttgccgaggtgaagaagttcaccctgtttccaagcgtggacacaaagaagatcgagctgaacatcgccccccagctgctggagatcgtgggcaaggccatcaaggagatcgagaagtccgagaacaagaat

SEQ ID NO: 78 - protein, 1127 aa, synthetic construct
  REGION 1..1127: note = PbuCas13b amino acid sequence
MQKQDKLFVDRKKNAIFAFPKYITIMENKEKPEPIYYELTDKHFWAAFLNLARHNVYTTINHINRRLEIAELKDDGYMMGIKGSWNEQAKKLDKKVRLRDLIMKHFPFLEAAAYEMTNSKSPNNKEQREKEQSEALSLNNLKNVLFIFLEKLQVLRNYYSHYKYSEESPKPIFETSLLKNMYKVFDANVRLVKRDYMHHENIDMQRDFTHLNRKKQVGRTKNIIDSPNFHYHFADKEGNMTIAGLLFFVSLFLDKKDAIWMQKKLKGFKDGRNLREQMTNEVFCRSRISLPKLKLENVQTKDWMQLDMLNELVRCPKSLYERLREKDRESFKVPFDIFSDDYNAEEEPFKNTLVRHQDRFPYFVLRYFDLNEIFEQLRFQIDLGTYHFSIYNKRIGDEDEVRHLTHHLYGFARIQDFAPQNQSEEWRKLVKDLDHFETSQEPYISKTAPHYHLENEKIGIKFCSAHNNLFPSLQTDKTCNGRSKFNLGTQFTAEAFLSVHELLPMMFYYLLLTKDYSRKESADKVEGIIRKEISNIYAIYDAFANNEINSIADLTRRLQNTNILQGHLPKQMISILKGRQKDMGKEAERKIGEMIDDTQRRLDLLCKQTNQKIRIGKRNAGLLKSGKIADWLVNDMMRFQPVQKDQNNIPINNSKANSTEYRMLQRALALFGSENFRLKAYFNQMNLVGNDNPHPFLAETQWEHQTNILSFYRNYLEARKKYLKGLKPQNWKQYQHFLILKVQKTNRNTLVTGWKNSFNLPRGIFTQPIREWFEKHNNSKRIYDQILSFDRVGFVAKAIPLYFAEEYKDNVQPFYDYPFNIGNRLKPKKRQFLDKKERVELWQKNKELFKNYPSEKKKTDLAYLDFLSWKKFERELRLIKNQDIVTWLMFKELFNMATVEGLKIGEIHLRDIDTNTANEESNNILNRIMPMKLPVKTYETDNKGNILKERPLATFYIEETETKVLKQGNFKALVKDRRLNGLFSFAETTDLNLEEHPISKLSVDLELIKYQTTRISIFEMTLGLEKKLIDKYSTLPTDSFRNMLERWLQCKANRPELKNYVNSLIAVRNAFSHNQYPMYDATLFAEVKKFTLFPSVDTKKIELNIAPQLLEIVGKAIKEIEKSENKN

SEQ ID NO: 79 - DNA, 36 bp, synthetic construct
  misc_feature 1..36: note = PbuCas13b direct repeat
  source 1..36: mol_type = other DNA
gttgcatctgccttctttttgaaaggtaaaaacaac

SEQ ID NO: 80 - DNA, 2859 bp, synthetic construct
  misc_feature 1..2859: note = AspCas13b nucleic acid sequence
  source 1..2859: mol_type = other DNA
atgtccaatgagatcggcgccttcagagagcaccagtttgcctacgccccaggaaacgagaagcaggaggaggccaccttcgccacatacttcaacctggccctgtctaatgtggagggcatgatgttcggagaggtggagtctaacccagacaagatcgagaagagcctggataccctgccccctgccatcctgagacagatcgcctcctttatctggctgtctaaggaggaccaccccgataaggcctactccaccgaggaggtgaaagtgatcgtgacagacctggtgcggagactgtgcttctacaggaattatttcagccactgtttctatctggatacacagtacttttattccgacgagctggtggataccacagccatcggcgagaagctgccttacaatttccaccactttatcaccaacagactgttcaggtatagcctgccagagatcacactgtttcgctggaacgagggcgagcggaagtacgagatcctgcgggacggcctgatcttcttttgctgtctgttcctgaagaggggacaggcagagcggtttctgaatgagctgagattctttaagaggaccgacgaggagggccgcatcaagcggaccatcttcacaaagtattgcacaagagagtctcacaagcacatcggcatcgaggagcaggatttcctgatctttcaggacatcatcggcgatctgaatagggtgcctaaggtgtgcgacggcgtggtggatctgagcaaggagaacgagagatacatcaagaacagggagacaagcaatgagtctgatgagaacaaggcccggtatagactgctgatcagggagaaggacaagtttccctactatctgatgcgctacatcgtggatttcggcgtgctgccttgcatcacctttaagcagaacgactacagcacaaaggagggccggggccagttccactatcaggatgcagcagtggcacaggaggagaggtgttacaattttgtggtgcgcaacggcaacgtgtactattcctatatgcctcaggcccagaacgtggtgcggatcagcgagctgcagggcaccatctccgtggaggagctgagaaatatggtgtacgcctctatcaatggcaaggacgtgaacaagagcgtggagcagtacctgtatcacctgcacctgctgtatgagaagatcctgaccatctccggccagacaatcaaggagggccgggtggacgtggaggattacagacctctgctggataagctgctgctgcgcccagcctctaacggcgaggagctgaggcgcgagctgaggaagctgctgccaaagcgcgtgtgcgacctgctgagcaatcggttcgattgttctgagggcgtgagcgccgtggagaagaggctgaaggccatcctgctgcgccacgagcagctgctgctgagccagaaccccgccctgcacatcgacaagatcaagtccgtgatcgattacctgtatctgttcttttctgacgatgagaagttccggcagcagcctaccgagaaggcccacagaggcctgaaggacgaggagtttcagatgtaccactatctggtgggcgactacgattctcacccactggccctgtggaaggagctggaggccagcggccggctgaagccagagatgagaaagctgaccagcgccacatccctgcacggcctgtatatgctgtgcctgaagggcaccgtggagtggtgtcggaagcagctgatgtccatcggcaagggcacagccaaggtggaggccatcgccgacagagtgggcctgaagctgtacgataagctgaaggagtatacccctgagcagctggagagggaggtgaagctggtggtcatgcacggatacgctgccgccgccaccccaaagccaaaggcacaggcagcaatcccatctaagctgacagagctgcggttctattcctttctgggcaagagagagatgtctttcgccgcctttatccggcaggacaagaaggcccagaagctgtggctgagaaatttctataccgtggagaacatcaagacactgcagaagaggcaggctgccgccgacgcagcctgcaagaagctgtataacctggtgggagaggtggagcgggtgcacaccaacgataaggtgctggtgctggtggcccagaggtatcgcgagcggctgctgaatgtgggcagcaagtgtgccgtgacactggacaaccccgagaggcagcagaagctggccgacgtgtacgaggtgcagaacgcctggctgtccatccgcttcgacgatctggactttaccctgacacacgtgaacctgtctaatctgaggaaggcctacaatctgatcccccgcaagcacatcctggccttcaaggagtatctggacaatagggtgaagcagaagctgtgcgaggagtgtcgcaacgtgcggagaaaggaggatctgtgcacctgctgttcccctcgctactctaatctgacaagctggctgaaggagaaccactccgagagctccatcgagagggaggccgccaccatgatgctgctggacgtggagcgcaagctgctgagcttcctgctggatgagaggcgcaaggccatcatcgagtacggcaagttcatcccattttccgccctggtgaaggagtgccggctggccgacgcaggcctgtgcggcatcagaaatgacgtgctgcacgataacgtgatcagctacgccgatgccatcggcaagctgagcgcctattttcccaaggaggcctccgaggccgtggagtacatccggagaacaaaggaggtgcgggagcagaggcgcgaggagctgatggccaactctagccag

SEQ ID NO: 81 - protein, 953 aa, synthetic construct
  REGION 1..953: note = AspCas13b amino acid sequence
MSNEIGAFREHQFAYAPGNEKQEEATFATYFNLALSNVEGMMFGEVESNPDKIEKSLDTLPPAILRQIASFIWLSKEDHPDKAYSTEEVKVIVTDLVRRLCFYRNYFSHCFYLDTQYFYSDELVDTTAIGEKLPYNFHHFITNRLFRYSLPEITLFRWNEGERKYEILRDGLIFFCCLFLKRGQAERFLNELRFFKRTDEEGRIKRTIFTKYCTRESHKHIGIEEQDFLIFQDIIGDLNRVPKVCDGVVDLSKENERYIKNRETSNESDENKARYRLLIREKDKFPYYLMRYIVDFGVLPCITFKQNDYSTKEGRGQFHYQDAAVAQEERCYNFVVRNGNVYYSYMPQAQNVVRISELQGTISVEELRNMVYASINGKDVNKSVEQYLYHLHLLYEKILTISGQTIKEGRVDVEDYRPLLDKLLLRPASNGEELRRELRKLLPKRVCDLLSNRFDCSEGVSAVEKRLKAILLRHEQLLLSQNPALHIDKIKSVIDYLYLFFSDDEKFRQQPTEKAHRGLKDEEFQMYHYLVGDYDSHPLALWKELEASGRLKPEMRKLTSATSLHGLYMLCLKGTVEWCRKQLMSIGKGTAKVEAIADRVGLKLYDKLKEYTPEQLEREVKLVVMHGYAAAATPKPKAQAAIPSKLTELRFYSFLGKREMSFAAFIRQDKKAQKLWLRNFYTVENIKTLQKRQAAADAACKKLYNLVGEVERVHTNDKVLVLVAQRYRERLLNVGSKCAVTLDNPERQQKLADVYEVQNAWLSIRFDDLDFTLTHVNLSNLRKAYNLIPRKHILAFKEYLDNRVKQKLCEECRNVRRKEDLCTCCSPRYSNLTSWLKENHSESSIEREAATMMLLDVERKLLSFLLDERRKAIIEYGKFIPFSALVKECRLADAGLCGIRNDVLHDNVISYADAIGKLSAYFPKEASEAVEYIRRTKEVREQRREELMANSSQ

SEQ ID NO: 82 - DNA, 36 bp, synthetic construct
  misc_feature 1..36: note = AspCas13b direct repeat
  source 1..36: mol_type = other DNA
gctgttatatccttacctttgtaagggaagtacagc

SEQ ID NO: 83 - DNA, 3966 bp, synthetic construct
  misc_feature 1..3966: note = PsmCas13b nucleic acid sequence
  source 1..3966: mol_type = other DNA
atgtctaaggagtgcaagaagcagcgccaggagaagaagcggagactgcagaaggccaatttctccatctctctgaccggcaagcacgtgttcggcgcctactttaatatggccagaacaaactttgtgaaaaccatcaactacatcctgcctatcgccggcgtgaggggcaactatagcgagaatcagatcaacaagatgctgcacgcactgttcctgatccaggcaggacgcaatgaggagctgaccacagagcagaagcagtgggagaagaagctgcggctgaacccagagcagcagaccaagtttcagaagctgctgttcaagcactttccagtgctgggaccaatgatggcagacgtggcagatcacaaggcctatctgaacaagaagaagagcaccgtgcagacagaggacgagacattcgccatgctgaagggcgtgtccctggccgactgcctggatatcatctgtctgatggccgataccctgacagagtgtagaaacttctacacacacaaggacccttataacaagccaagccagctggccgatcagtacctgcaccaggagatgatcgccaagaagctggacaaggtggtggtggcctccaggcgcatcctgaaggatagggagggcctgtctgtgaatgaggtggagttcctgaccggcatcgatcacctgcaccaggaggtgctgaaggacgagttcggcaacgccaaggtgaaggatggcaaagtgatgaaaaccttcgtggagtacgacgacttctacttcaagatctccggcaagcggctggtgaatggctacacagtgaccacaaaggacgataagcccgtgaatgtgaacaccatgctgcctgccctgtctgacttcggcctgctgtacttctgcgtgctgtttctgagcaagccctatgccaagctgtttatcgatgaggtgcgcctgttcgagtactctccttttgacgataaggagaacatgatcatgtctgagatgctgagcatctatcggatcagaacaccccggctgcacaagatcgactcccacgattctaaggccaccctggccatggacatcttcggcgagctgcggagatgtcctatggagctgtataacctgctggacaagaacgccggccagccattctttcacgatgaggtgaagcacccaaactctcacacccccgacgtgagcaagcggctgagatacgacgatcgctttcctacactggccctgcggtatatcgatgagacagagctgttcaagcggatcagatttcagctgcagctgggcagcttccgctacaagttttatgataaggagaattgcatcgacggccgggtgagagtgaggcgcatccagaaggagatcaacggctacggcagaatgcaggaggtggccgacaagaggatggataagtggggcgacctgatccagaagagggaggagcgcagcgtgaagctggagcacgaggagctgtatatcaatctggaccagttcctggaggacaccgccgattccacaccttacgtgaccgatagacggcccgcctataatatccacgccaaccggatcggcctgtactgggaggactcccagaatcctaagcagtacaaggtgtttgatgagaacggcatgtatatccccgagctggtggtgaccgaggacaagaaggcccctatcaagatgccagcacctaggtgtgcactgagcgtgtacgacctgccagccatgctgttctacgagtatctgcgggagcagcaggataacgagtttcccagcgccgagcaggtcatcatcgagtacgaggacgattatcgcaagttctttaaggccgtggccgagggcaagctgaagcccttcaagcggcccaaggagttcagggactttctgaagaaggagtacccaaagctgcggatggccgatatccccaagaagctgcagctgtttctgtgcagccacggcctgtgctacaacaataagcccgagacagtgtatgagcggctggacagactgaccctgcagcacctggaggagagagagctgcacatccagaacaggctggagcactaccagaaggacagagatatgatcggcaataaggacaaccagtatggcaagaagagcttctccgatgtgcgccacggcgccctggccagatacctggcacagagcatgatggagtggcagcccacaaagctgaaggataaggagaagggccacgacaagctgaccggcctgaattataacgtgctgacagcctacctggcaacctatggccacccacaggtgcctgaggagggcttcacccctagaacactggagcaggtgctgatcaatgcccacctgatcggcggctccaatccacacccctttatcaacaaggtgctggccctgggcaataggaacatcgaggagctgtacctgcactatctggaggaggagctgaagcacatcaggtcccgcatccagtctctgagctccaaccctagcgacaaggccctgtccgccctgccattcatccaccacgatcggatgagataccacgagcgcaccagcgaggagatgatggccctggccgcacggtataccacaatccagctgccagacggcctgtttacaccctacatcctggagatcctgcagaagcactataccgagaacagcgatctgcagaacgccctgtctcaggacgtgcctgtgaagctgaatccaacctgcaacgccgcctacctgatcacactgttctatcagaccgtgctgaaggacaatgcccagcccttctacctgtccgataagacctatacaagaaacaaggacggcgagaaggccgagtctttcagctttaagagggcctacgagctgttctctgtgctgaacaataacaagaaggacacattcccctttgagatgatccctctgtttctgaccagcgatgagatccaggagagactgtccgccaagctgctggacggcgatggcaatcctgtgccagaagtgggagagaagggcaagccagccacagacagccagggcaacaccatctggaagaggcgcatctactccgaggtggacgattatgccgagaagctgaccgaccgcgatatgaagatctccttcaagggcgagtgggagaagctgcctcggtggaagcaggataagatcatcaagcggcgggacgagacacggcggcagatgcgggacgagctgctgcagaggatgccacgctacatccgggacatcaaggataatgagaggacactgcggagatataagacccaggatatggtgctgttcctgctggccgagaagatgtttaccaatatcatctccgagcagtctagcgagttcaactggaagcagatgaggctgtctaaggtgtgcaacgaggccttcctgagacagaccctgaccttccgggtgcccgtgacagtgggcgagacaacaatctacgtggagcaggagaatatgtctctgaagaactacggcgagttctatcgctttctgacagacgatcggctgatgagcctgctgaataacatcgtggagacactgaagcccaatgagaacggcgacctggtcatcagacacacagacctgatgagcgagctggccgcctacgaccagtataggtctaccatcttcatgctgatccagagcatcgagaacctgatcatcacaaataacgccgtgctggacgatccagacgccgatggcttctgggtgagagaggatctgcccaagaggaataactttgcctccctgctggagctgatcaatcagctgaataacgtggagctgaccgacgatgagcgcaagctgctggtggccatccggaacgccttcagccacaattcctacaacatcgatttttccctgatcaaggacgtgaagcacctgcccgaggtggccaagggcatcctgcagcacctgcagtctatgctgggcgtggagatcaccaag

SEQ ID NO: 84 - protein, 1322 aa, synthetic construct
  REGION 1..1322: note = PsmCas13b amino acid sequence
MSKECKKQRQEKKRRLQKANFSISLTGKHVFGAYFNMARTNFVKTINYILPIAGVRGNYSENQINKMLHALFLIQAGRNEELTTEQKQWEKKLRLNPEQQTKFQKLLFKHFPVLGPMMADVADHKAYLNKKKSTVQTEDETFAMLKGVSLADCLDIICLMADTLTECRNFYTHKDPYNKPSQLADQYLHQEMIAKKLDKVVVASRRILKDREGLSVNEVEFLTGIDHLHQEVLKDEFGNAKVKDGKVMKTFVEYDDFYFKISGKRLVNGYTVTTKDDKPVNVNTMLPALSDFGLLYFCVLFLSKPYAKLFIDEVRLFEYSPFDDKENMIMSEMLSIYRIRTPRLHKIDSHDSKATLAMDIFGELRRCPMELYNLLDKNAGQPFFHDEVKHPNSHTPDVSKRLRYDDRFPTLALRYIDETELFKRIRFQLQLGSFRYKFYDKENCIDGRVRVRRIQKEINGYGRMQEVADKRMDKWGDLIQKREERSVKLEHEELYINLDQFLEDTADSTPYVTDRRPAYNIHANRIGLYWEDSQNPKQYKVFDENGMYIPELVVTEDKKAPIKMPAPRCALSVYDLPAMLFYEYLREQQDNEFPSAEQVIIEYEDDYRKFFKAVAEGKLKPFKRPKEFRDFLKKEYPKLRMADIPKKLQLFLCSHGLCYNNKPETVYERLDRLTLQHLEERELHIQNRLEHYQKDRDMIGNKDNQYGKKSFSDVRHGALARYLAQSMMEWQPTKLKDKEKGHDKLTGLNYNVLTAYLATYGHPQVPEEGFTPRTLEQVLINAHLIGGSNPHPFINKVLALGNRNIEELYLHYLEEELKHIRSRIQSLSSNPSDKALSALPFIHHDRMRYHERTSEEMMALAARYTTIQLPDGLFTPYILEILQKHYTENSDLQNALSQDVPVKLNPTCNAAYLITLFYQTVLKDNAQPFYLSDKTYTRNKDGEKAESFSFKRAYELFSVLNNNKKDTFPFEMIPLFLTSDEIQERLSAKLLDGDGNPVPEVGEKGKPATDSQGNTIWKRRIYSEVDDYAEKLTDRDMKISFKGEWEKLPRWKQDKIIKRRDETRRQMRDELLQRMPRYIRDIKDNERTLRRYKTQDMVLFLLAEKMFTNIISEQSSEFNWKQMRLSKVCNEAFLRQTLTFRVPVTVGETTIYVEQENMSLKNYGEFYRFLTDDRLMSLLNNIVETLKPNENGDLVIRHTDLMSELAAYDQYRSTIFMLIQSIENLIITNNAVLDDPDADGFWVREDLPKRNNFASLLELINQLNNVELTDDERKLLVAIRNAFSHNSYNIDFSLIKDVKHLPEVAKGILQHLQSMLGVEITK

SEQ ID NO: 85 - DNA, 36 bp, synthetic construct
  misc_feature 1..36: note = PsmCas13b direct repeat
  source 1..36: mol_type = other DNA
gttgtagaagcttatcgtttggataggtatgacaac

SEQ ID NO: 86 - DNA, 3285 bp, synthetic construct
  misc_feature 1..3285: note = RanCas13b nucleic acid sequence
  source 1..3285: mol_type = other DNA
atggagaagcccctgctgcctaacgtgtataccctgaagcacaagttcttttggggcgccttcctgaatatcgcccggcacaacgcctttatcaccatctgccacatcaatgagcagctgggcctgaaaacccccagcaacgacgataagatcgtggacgtggtgtgcgagacatggaacaatatcctgaacaatgaccacgatctgctgaagaagtcccagctgaccgagctgatcctgaagcacttcccttttctgacagccatgtgctaccacccccctaagaaggagggcaagaagaagggccaccagaaggagcagcagaaggagaaggagagcgaggcacagtcccaggcagaggccctgaacccatccaagctgatcgaggccctggagatcctggtgaatcagctgcactctctgcggaactactatagccactataagcacaagaagcccgacgccgagaaggatatcttcaagcacctgtataaggccttcgacgcctctctgagaatggtgaaggaggattataaggcccacttcaccgtgaatctgacaagggactttgcccacctgaaccgcaagggcaagaacaagcaggacaatcccgacttcaacagatacagattcgagaaggatggcttctttaccgagtctggcctgctgttctttacaaatctgtttctggacaagagggatgcctattggatgctgaagaaggtgtccggcttcaaggcctctcacaagcagcgcgagaagatgaccacagaggtgttttgcaggagccgcatcctgctgcccaagctgaggctggagtcccgctacgaccacaaccagatgctgctggatatgctgtctgagctgagcagatgtcctaagctgctgtatgagaagctgagcgaggagaataagaagcacttccaggtggaggccgacggctttctggatgagatcgaggaggagcagaacccattcaaggacaccctgatccggcaccaggatagattcccctactttgccctgaggtatctggacctgaatgagtccttcaagtctatccgctttcaggtggatctgggcacataccactattgtatctacgacaagaagatcggcgatgagcaggagaagaggcacctgacccgcacactgctgtccttcggccggctgcaggactttaccgagatcaacagaccccaggagtggaaggccctgaccaaggacctggattacaaggagacatccaatcagcctttcatctctaagaccacaccacactatcacatcaccgacaacaagatcggctttcggctgggcacaagcaaggagctgtacccctccctggagatcaaggatggcgccaatagaatcgccaagtacccttataactccggcttcgtggcccacgcctttatctctgtgcacgagctgctgcctctgatgttctaccagcacctgaccggcaagagcgaggacctgctgaaggagacagtgcggcacatccagagaatctataaggacttcgaggaggagcggatcaataccatcgaggatctggagaaggcaaaccagggcagactgccactgggagcctttcccaagcagatgctgggcctgctgcagaataagcagcctgatctgtccgagaaggccaagatcaagatcgagaagctgatcgccgagacaaagctgctgtctcacaggctgaacacaaagctgaagagctccccaaagctgggcaagcggagagagaagctgatcaagacaggcgtgctggccgactggctggtgaaggatttcatgcgctttcagcccgtggcctacgacgcccagaatcagcctatcaagtctagcaaggccaactccaccgagttctggtttatcaggcgcgccctggccctgtacggaggagagaagaataggctggagggctatttcaagcagaccaacctgatcggcaacacaaatccacaccccttcctgaacaagtttaattggaaggcctgcaggaatctggtggatttctaccagcagtatctggagcagcgcgagaagtttctggaggccatcaagaaccagccatgggagccctaccagtattgcctgctgctgaagatccctaaggagaacaggaagaatctggtgaagggatgggagcagggaggaatctctctgccacggggcctgtttaccgaggccatcagagagacactgtctgaggacctgatgctgagcaagccaatccgcaaggagatcaagaagcacggccgggtgggcttcatcagcagagccatcaccctgtactttaaggagaagtatcaggataagcaccagagcttctacaatctgtcctataagctggaggcaaaggcaccactgctgaagcgggaggagcactacgagtattggcagcagaacaagcctcagtctccaaccgagagccagaggctggagctgcacacaagcgaccgctggaaggattacctgctgtataagagatggcagcacctggagaagaagctgcggctgtaccggaatcaggacgtgatgctgtggctgatgaccctggagctgacaaagaaccacttcaaggagctgaacctgaattatcaccagctgaagctggagaatctggccgtgaacgtgcaggaggccgatgccaagctgaaccctctgaatcagaccctgcccatggtgctgcctgtgaaggtgtaccctgccacagcctttggcgaggtgcagtaccacaagaccccaatcaggacagtgtatatccgcgaggagcacaccaaggccctgaagatgggcaacttcaaggccctggtgaaggaccggagactgaatggcctgttctcttttatcaaggaggagaacgatacacagaagcacccaatcagccagctgcggctgaggcgcgagctggagatctaccagtctctgagagtggacgccttcaaggagacactgagcctggaggagaagctgctgaataagcacacatccctgtcctctctggagaacgagtttagggccctgctggaggagtggaagaaggagtatgccgccagctccatggtgacagacgagcacatcgccttcatcgccagcgtgcgcaatgccttctgccacaaccagtaccccttctacaaggaggccctgcacgcccctatcccactgttcaccgtggcccagcccaccacagaggagaaggatggcctgggaatcgcagaggccctgctgaaggtgctgagggagtactgtgagatcgtgaagtcccagatc

SEQ ID NO: 87 - protein, 1095 aa, synthetic construct
  REGION 1..1095: note = RanCas13b amino acid sequence
MEKPLLPNVYTLKHKFFWGAFLNIARHNAFITICHINEQLGLKTPSNDDKIVDVVCETWNNILNNDHDLLKKSQLTELILKHFPFLTAMCYHPPKKEGKKKGHQKEQQKEKESEAQSQAEALNPSKLIEALEILVNQLHSLRNYYSHYKHKKPDAEKDIFKHLYKAFDASLRMVKEDYKAHFTVNLTRDFAHLNRKGKNKQDNPDFNRYRFEKDGFFTESGLLFFTNLFLDKRDAYWMLKKVSGFKASHKQREKMTTEVFCRSRILLPKLRLESRYDHNQMLLDMLSELSRCPKLLYEKLSEENKKHFQVEADGFLDEIEEEQNPFKDTLIRHQDRFPYFALRYLDLNESFKSIRFQVDLGTYHYCIYDKKIGDEQEKRHLTRTLLSFGRLQDFTEINRPQEWKALTKDLDYKETSNQPFISKTTPHYHITDNKIGFRLGTSKELYPSLEIKDGANRIAKYPYNSGFVAHAFISVHELLPLMFYQHLTGKSEDLLKETVRHIQRIYKDFEEERINTIEDLEKANQGRLPLGAFPKQMLGLLQNKQPDLSEKAKIKIEKLIAETKLLSHRLNTKLKSSPKLGKRREKLIKTGVLADWLVKDFMRFQPVAYDAQNQPIKSSKANSTEFWFIRRALALYGGEKNRLEGYFKQTNLIGNTNPHPFLNKFNWKACRNLVDFYQQYLEQREKFLEAIKNQPWEPYQYCLLLKIPKENRKNLVKGWEQGGISLPRGLFTEAIRETLSEDLMLSKPIRKEIKKHGRVGFISRAITLYFKEKYQDKHQSFYNLSYKLEAKAPLLKREEHYEYWQQNKPQSPTESQRLELHTSDRWKDYLLYKRWQHLEKKLRLYRNQDVMLWLMTLELTKNHFKELNLNYHQLKLENLAVNVQEADAKLNPLNQTLPMVLPVKVYPATAFGEVQYHKTPIRTVYIREEHTKALKMGNFKALVKDRRLNGLFSFIKEENDTQKHPISQLRLRRELEIYQSLRVDAFKETLSLEEKLLNKHTSLSSLENEFRALLEEWKKEYAASSMVTDEHIAFIASVRNAFCHNQYPFYKEALHAPIPLFTVAQPTTEEKDGLGIAEALLKVLREYCEIVKSQI

SEQ ID NO: 88 - DNA, 36 bp, synthetic construct
  misc_feature 1..36: note = RanCas13b direct repeat
  source 1..36: mol_type = other DNA
gttgggactgctctcactttgaagggtattcacaac

SEQ ID NO: 89 - DNA, 3372 bp, synthetic construct
  misc_feature 1..3372: note = PauCas13b nucleic acid sequence
  source 1..3372: mol_type = other DNA
atggaggacgataagaaaaccacaggcagcatctcctacgagctgaaggacaagcacttctgggccgcctttctgaacctggcccgccacaacgtgtatatcaccatcaaccacatcaataagctgctggagatccgggagatcgacaacgatgagaaggtgctggacatcaagaccctgtggcagaagggcaacaaggatctgaatcagaaggccaggctgcgcgagctgatgacaaagcacttccccttcctggagacagccatctacacaaagaacaaggaggataagaaggaggtgaagcaggagaagcaggccgaggcccagtctctggagagcctgaaggactgcctgttcctgtttctggataagctgcaggaggcccgcaattactatagccactacaagtattccgagttctctaaggagccagagtttgaggagggcctgctggagaagatgtataacatctttggcaacaatatccagctggtcatcaacgactatcagcacaacaaggatatcaatcccgacgaggatttcaagcacctggaccggaagggccagttcaagtactcctttgccgataacgagggcaatatcaccgagtccggcctgctgttcttcgtgagcctgttcctggagaagaaggacgccatctggatgcagcagaagctgaacggctttaaggataacctggagaacaagaagaagatgacacacgaggtgttctgccggagcagaatcctgatgcccaagctgcgcctggagagcacccagacacaggactggattctgctggacatgctgaatgagctgatccggtgtcctaagtccctgtacgagagactgcagggcgacgatagggagaagttcaaggtgccatttgaccccgccgacgaggattacaacgccgagcaggagccttttaagaataccctgatcagacaccaggacaggttcccatactttgtgctgagatacttcgattacaacgagatcttcaagaatctgaggtttcagatcgacctgggcacctaccacttctctatctataagaagctgatcggcggccagaaggaggatcgccacctgacacacaagctgtacggcttcgagcgcatccaggagtttgccaagcagaaccggcctgacgagtggaaggccatcgtgaaggacctggatacctacgagacaagcaacaagcggtatatctccgagacaacacctcactatcacctggagaatcagaagatcggcatcaggtttcgcaacggcaataaggagatctggcccagcctgaaaaccaacgacgagaacaatgagaagtctaagtacaagctggataagcagtatcaggccgaggcctttctgtccgtgcacgagctgctgcccatgatgttctactatctgctgctgaagaaggagaagcctaacaatgacgagatcaatgcctctatcgtggagggcttcatcaagcgggagatcagaaacatcttcaagctgtacgacgcctttgccaatggcgagatcaacaatatcgacgatctggagaagtattgcgccgataagggcatccccaagagacacctgcctaagcagatggtggccatcctgtacgacgagcacaaggatatggtgaaggaggccaagaggaagcagaaggagatggtgaaggataccaagaagctgctggccaccctggagaagcagacacagaaggagaaggaggacgatggcagaaacgtgaagctgctgaagagcggcgagatcgccagatggctggtgaatgacatgatgaggttccagcccgtgcagaaggataacgagggcaagcctctgaacaatagcaaggccaattccaccgagtaccagatgctgcagcggagcctggccctgtataacaatgaggagaagcccacaaggtattttcgccaggtgaacctgatcgagagcaacaatccccaccctttcctgaagtggaccaagtgggaggagtgtaacaacatcctgaccttttactactcttacctgacaaagaagatcgagttcctgaacaagctgaagccagaggactggaagaagaatcagtattttctgaagctgatggagcccaagaccaacagagagacactggtgcagggctggaagaacggctttaatctgcctaggggcatcttcacagagccaatccgcgagtggttcaagcggcaccagaacaatagcaaggagtacgagaaggtggaggccctggacagagtgggcctggtgaccaaagtgatccccctgttctttaaggaggagtacttcaaggataaggaggagaacttcaaggaggacacacagaaggagatcaacgattgcgtgcagccattctacaattttccctataacgtgggcaatatccacaagcctaaggagaaggactttctgcaccgggaggagagaatcgagctgtgggacaagaagaaggataagttcaagggctacaaggagaagatcaagagcaagaagctgaccgagaaggacaaggaggagttcaggtcttatctggagtttcagagctggaacaagttcgagagggagctgcgcctggtgcggaatcaggatatcgtgacatggctgctgtgcaaggagctgatcgacaagctgaagatcgatgagctgaacatcgaggagctgaagaagctgagactgaacaatatcgacaccgatacagccaagaaggagaagaacaatatcctgaatagagtgatgcctatggagctgccagtgaccgtgtacgagatcgacgattcccacaagatcgtgaaggacaagccactgcacaccatctatatcaaggaggccgagacaaagctgctgaagcagggcaacttcaaggccctggtgaaggatcggagactgaatggcctgttcagcttcgtgaaaaccaacagcgaggccgagagcaagcgcaatcctatctccaagctgcgggtggagtacgagctgggcgagtatcaggaggcccggatcgagatcatccaggacatgctggccctggaggagaagctgatcaacaagtacaaggatctgccaacaaacaagttttccgagatgctgaacagctggctggagggcaaggacgaggccgataaggcccgctttcagaatgacgtggatttcctgatcgccgtgcggaacgccttctcccacaatcagtacccaatgcacaacaagatcgagtttgccaatatcaagcccttcagcctgtataccgccaacaattccgaggagaagggcctgggcatcgccaaccagctgaaggacaagaccaaggagacaacagataagatcaagaagatcgagaagcccatcgagacaaaggag

SEQ ID NO: 90 - protein, 1124 aa, synthetic construct
  REGION 1..1124: note = PauCas13 amino acid sequence
MEDDKKTTGSISYELKDKHFWAAFLNLARHNVYITINHINKLLEIREIDNDEKVLDIKTLWQKGNKDLNQKARLRELMTKHFPFLETAIYTKNKEDKKEVKQEKQAEAQSLESLKDCLFLFLDKLQEARNYYSHYKYSEFSKEPEFEEGLLEKMYNIFGNNIQLVINDYQHNKDINPDEDFKHLDRKGQFKYSFADNEGNITESGLLFFVSLFLEKKDAIWMQQKLNGFKDNLENKKKMTHEVFCRSRILMPKLRLESTQTQDWILLDMLNELIRCPKSLYERLQGDDREKFKVPFDPADEDYNAEQEPFKNTLIRHQDRFPYFVLRYFDYNEIFKNLRFQIDLGTYHFSIYKKLIGGQKEDRHLTHKLYGFERIQEFAKQNRPDEWKAIVKDLDTYETSNKRYISETTPHYHLENQKIGIRFRNGNKEIWPSLKTNDENNEKSKYKLDKQYQAEAFLSVHELLPMMFYYLLLKKEKPNNDEINASIVEGFIKREIRNIFKLYDAFANGEINNIDDLEKYCADKGIPKRHLPKQMVAILYDEHKDMVKEAKRKQKEMVKDTKKLLATLEKQTQKEKEDDGRNVKLLKSGEIARWLVNDMMRFQPVQKDNEGKPLNNSKANSTEYQMLQRSLALYNNEEKPTRYFRQVNLIESNNPHPFLKWTKWEECNNILTFYYSYLTKKIEFLNKLKPEDWKKNQYFLKLMEPKTNRETLVQGWKNGFNLPRGIFTEPIREWFKRHQNNSKEYEKVEALDRVGLVTKVIPLFFKEEYFKDKEENFKEDTQKEINDCVQPFYNFPYNVGNIHKPKEKDFLHREERIELWDKKKDKFKGYKEKIKSKKLTEKDKEEFRSYLEFQSWNKFERELRLVRNQDIVTWLLCKELIDKLKIDELNIEELKKLRLNNIDTDTAKKEKNNILNRVMPMELPVTVYEIDDSHKIVKDKPLHTIYIKEAETKLLKQGNFKALVKDRRLNGLFSFVKTNSEAESKRNPISKLRVEYELGEYQEARIEIIQDMLALEEKLINKYKDLPTNKFSEMLNSWLEGKDEADKARFQNDVDFLIAVRNAFSHNQYPMHNKIEFANIKPFSLYTANNSEEKGLGIANQLKDKTKETTDKIKKIEKPIETKE

SEQ ID NO: 91 - DNA, 36 bp, synthetic construct
  misc_feature 1..36: note = PauCas13b direct repeat
  source 1..36: mol_type = other DNA
gttgtatctgccttctgtttgaaaggtaaaaacaac

SEQ ID NO: 92 - DNA, 1422 bp, synthetic construct
  misc_feature 1..1422: note = CMV-BD-2xms2-PPT-exon-MMP9_intron2-2xms2-BD-bGHpA
  source 1..1422: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcggatcttccatttcgggtgtcgtgacgtacgaatacgatctagaccctgcatcctctctgggacactcaacaccctcacggacagcccgaattcgacccaagctgacgggagcacatgaggatcacccatgtgccacgagcgacatgaggatcacccatgtcgctttcactagtctgtggtgtgatatccatggcggcctacttatcctgtcccttttttttccacaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgagccggggctgtgaaagctgcgggatggagcgagaaggcctgatctggctcttgagcctgcaacggagcacatgaggatcacccatgtgccacgagcgacatgaggatcacccatgtcgaattcgacccaagctgactgactgagacgagttaactgtaccgtctccgcttctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 93 - DNA, 5403 bp, synthetic construct
  misc_feature 1..5403: note = PspCas13b-ABI1-2a-PYL1-MS2 nucleic acid sequence
  source 1..5403: mol_type = other DNA
atgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaacgcatacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatgcaaacaattaccccgacaaaggcgtggtggaaatcaaggccctgcctgagatcgccatgagcatcaagaaggcctttggggagtacgccatcatgaagggaagcctgcaggcctatccctatgacgtgcccgattatgccagcctgggcagcggctcccccaagaaaaaacgcaaggtggaagatcctaagaaaaagcggaaagtggacggcattggtagtgggagcaacggaggtggaggctctggacctaggacgcgtgtgcctttgtatggttttacttcgatttgtggaagaagacctgagatggaagctgctgtttcgactataccaagattccttcaatcttcctctggttcgatgttagatggtcggtttgatcctcaatccgccgctcatttcttcggtgtttacgacggccatggcggttctcaggtagcgaactattgtagagagaggatgcatttggctttggcggaggagatagctaaggagaaaccgatgctctgcgatggtgatacgtggctggagaagtggaagaaagctcttttcaactcgttcctgagagttgactcggagattgagtcagttgcgccggagacggttgggtcaacgtcggtggttgccgttgttttcccgtctcacatcttcgtcgctaactgcggtgactctagagccgttctttgccgcggcaaaactgcacttccattatccgttgaccataaaccggatagagaagatgaagctgcgaggattgaagccgcaggagggaaagtgattcagtggaatggagctcgtgttttcggtgttctcgccatgtcgagatccattggcgatagatacttgaaaccatccatcattcctgatccggaagtgacggctgtgaagagagtaaaagaagatgattgtctgattttggcgagtgacggggtttgggatgtaatgacggatgaagaagcgtgtgagatggcaaggaagcggattctcttgtggcacaagaaaaacgcggtggctggggatgcatcgttgctcgcggatgagcggagaaaggaagggaaagatcctgcggcgatgtccgcggctgagtatttgtcaaagctggcgatacagagaggaagcaaagacaacataagtgtggtggtggttgatttgaagggatccggcgcaacaaacttctctctgctgaaacaagccggagatgtcgaagagaatcctggaccgatgggtgggggcgcgccaactcaagacgaattcacccaactctcccaatcaatcgccgagttccacacgtaccaactcggtaacggccgttgctcatctctcctagctcagcgaatccacgcgccgccggaaacagtatggtccgtggtgagacgtttcgataggccacagatttacaaacacttcatcaaaagctgtaacgtgagtgaagatttcgagatgcgagtgggatgcacgcgcgacgtgaacgtgataagtggattaccggcgaatacgtctcgagagagattagatctgttggacgatgatcggagagtgactgggtttagtataaccggtggtgaacataggctgaggaattataaatcggttacgacggttcatagatttgagaaagaagaagaagaagaaaggatctggaccgttgttttggaatcttatgttgttgatgtaccggaaggtaattcggaggaagatacgagattgtttgctgatacggttattagattgaatcttcagaaacttgcttcgatcactgaagctatgaacgaattctcaagcgctgcaggtactagtccaaagaagaagcggaaggtcggtggatccggaggaggtggaagcatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctac

SEQ ID NO: 94 - protein, 1801 aa, synthetic construct
  REGION 1..1801: note = PspCas13b-ABI1-2a-PYL1-MS2 amino acid seqeunce
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDANNYPDKGVVEIKALPEIAMSIKKAFGEYAIMKGSLQAYPYDVPDYASLGSGSPKKKRKVEDPKKKRKVDGIGSGSNGGGGSGPRTRVPLYGFTSICGRRPEMEAAVSTIPRFLQSSSGSMLDGRFDPQSAAHFFGVYDGHGGSQVANYCRERMHLALAEEIAKEKPMLCDGDTWLEKWKKALFNSFLRVDSEIESVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLKGSGATNFSLLKQAGDVEENPGPMGGGAPTQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMNEFSSAAGTSPKKKRKVGGSGGGGSMASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 95 - DNA, 3414 bp, synthetic construct
  misc_feature 1..3414: note = dRfxCas13d-MS2 nucleic acid sequence
  source 1..3414: mol_type = other DNA
atgagccccaagaagaagagaaaggtggaggccagcatcgaaaaaaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactggcgcactgggtggtcgctaacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcgcaaacaaggccgtcgccctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaattccggatccggacctaagaaaaagaggaaggtggcggccgcttacccatacgatgttccagattacgctgctggatccggaggaggtggaagcatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctac

SEQ ID NO: 96 - protein, 1138 aa, synthetic construct
  REGION 1..1138: note = dRfxCas13d-MS2 amino acid sequence
MSPKKKRKVEASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSGSGPKKKRKVAAAYPYDVPDYAAGSGGGGSMASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 97 - DNA, 3411 bp, synthetic construct
  misc_feature 1..3411: note = MS2-dRfxCas13d nucleic acid sequence
  source 1..3411: mol_type = other DNA
atggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacggatccggaggaggtggaagcagccccaagaagaagagaaaggtggaggccagcatcgaaaaaaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactggcgcactgggtggtcgctaacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcgcaaacaaggccgtcgccctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaattccggatccggacctaagaaaaagaggaaggtggcggccgcttacccatacgatgttccagattacgctgct

SEQ ID NO: 98 - protein, 1137 aa, synthetic construct
  REGION 1..1137: note = MS2-dRfxCas13d amino acid sequence
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYGSGGGGSSPKKKRKVEASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSGSGPKKKRKVAAAYPYDVPDYAA

SEQ ID NO: 99 - DNA, 57 bp, synthetic construct
  misc_feature 1..57: note = dRfxCas13d gRNA
  source 1..57: mol_type = other DNA
aacccctaccaactggtcggggtttgaaaccctgtggatgaagggacacacacatgc

SEQ ID NO: 100 - DNA, 5059 bp, synthetic construct
  misc_feature 1..5059: note = AAV2_ITR-CMV-dPspCas13b-MS2-bGHpA-AAV2_ITR
  source 1..5059: mol_type = other DNA
gctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagcgggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaacgcatacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatgcaaacaattaccccgacaaaggcgtggtggaaatcaaggccctgcctgagatcgccatgagcatcaagaaggcctttggggagtacgccatcatgaagggaagcctgcagccaaagaagaagcggaaggtcggtggatccggaggaggtggaagcatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctactaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtcgacatctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagc

SEQ ID NO: 101 - DNA, 7146 bp, synthetic construct
  misc_feature 1..7146: note = 5'LTR-EFS-dPspCas13b-MS2-2a-Blast-WPRE-3'LTR
  source 1..7146: mol_type = other DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaaggtaccgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagcgctgccaccatgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaacgcatacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatgcaaacaattaccccgacaaaggcgtggtggaaatcaaggccctgcctgagatcgccatgagcatcaagaaggcctttggggagtacgccatcatgaagggaagcctgcagccaaagaagaagcggaaggtcggtggatccggaggaggtggaagcatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacgcaacaaacttctctctgctgaaacaagccggagatgtcgaagagaatcctggaccgatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca